(19) United States
(12) Patent Application Publication
(10) Pub. No.: US 2022/0273184 A1
(43) Pub. Date: Sep. 1, 2022

(54) NEUROMELANIN-SENSITIVE MRI FOR ASSESSING PARKINSON'S DISEASE

(71) Applicants: Terran Biosciences, Inc., New York, NY (US); The Trustees of Columbia University in the City of New York, New York, NY (US); The Research Foundation for Mental Hygiene, Inc., Menands, NY (US)

(72) Inventors: Samuel CLARK, New York, NY (US); Guillermo HORGA HERNANDEZ, Brooklyn, NY (US); Clifford Mills CASSIDY, Ottawa (CA); Kenneth WENGLER, New York, NY (US)

(21) Appl. No.: 17/636,018

(22) PCT Filed: Aug. 17, 2020
(86) PCT No.: PCT/US2020/046686

§ 371 Date: Feb. 16, 2022

Related U.S. Application Data
(60) Provisional application No. 62/889,300, filed on Aug. 20, 2019.

(51) Int. Cl. A61B 5/055; A61B 5/00; G01N 33/68

(52) U.S. Cl. CPC A61B 5/055; A61B 5/4082; A61B 5/4842; G01N 33/6896; G01N 2800/50; G01R 33/5608

(57) ABSTRACT

A neuromelanin sensitive magnetic resonance imaging (“MRI”) technique, method and computer-accessible medium for measuring the extent of, providing a diagnosis of, monitoring the treatment of, assessing novel treatments for, or determining a prognosis related to Parkinson's disease.

### RELATED APPLICATIONS

This application claims priority to, and the benefit of, U.S. Provisional Application No. 62/889,300, filed on Aug. 20, 2019, the content of which is incorporated by reference in its entirety.

### GOVERNMENT SUPPORT

The work described herein was supported in whole, or in part, by National Institutes Health Grant Nos. R01MH114965, R01MH117323, R01DA020855, and UL1TR001873. Thus, the United States Government has certain rights to the invention.

### FIELD OF THE DISCLOSURE

The present disclosure relates generally to magnetic resonance imaging (“MRI”), and more specifically, to exemplary embodiments of an exemplary system, method and computer-accessible medium for a neuromelanin-sensitive MRI technique as a non-invasive measure of neurological conditions with an emphasis on Parkinson's Disease.

### BACKGROUND

Parkinson's disease, one of the two great neurodegenerative diseases of aging, is a progressive neurological disease affecting as many as 1,500,000 Americans. Parkinson's disease occurs when certain nerve cells (neurons) in the part of the brain called the substantia nigra die or become impaired. Normally, these cells produce a vital chemical known as dopamine. Dopamine allows smooth, coordinated function of the body's muscles and movement. When approximately 80% of the dopamine-producing cells are damaged, the symptoms of Parkinson's disease appear. Parkinson's disease affects both men and women in almost equal numbers. It shows no social, ethnic, economic or geographic boundaries. In the United States, it is estimated that 60,000 new cases are diagnosed each year. While the condition usually develops after the age of 65, 15% of those diagnosed are under 50. Idiopathic Parkinson's Disease is by far the most common, and includes the rare genetic forms caused by mutations in the genes for alpha-synuclein and parkin. Known environmental causes include the very rare cases of poisoning by MPTP (1-methyl-4-phenyl-4-propionoxypiperidine), carbon monoxide, and manganese.

The incidence of Parkinson's disease increases with age. The median age of onset for all forms of Parkinson syndrome is 61.6 years, with median idiopathic Parkinson's disease onset at 62.4 years. Onset before age 30 is rare, but up to 10% of cases of idiopathic Parkinson's Disease begin by age 40. In a recent study in the United States, the incidence of Parkinson's was 10.9 cases per 100,000 person years in the general population, and 49.7 per 100,000 person-years for these over age 50. The incidence is growing as the population ages. Prevalence is estimated to be approximately 300 per 100,000 in the United States and Canada, but with the important caveat that perhaps 40% of cases may be undiagnosed at any given time.

Symptoms such as bradykinesia are slowness in voluntary movements. It produces difficulty initiating movement as well as difficulty completing movement once it is in progress. The delayed transmission from the brain to the skeletal muscles, due to diminished dopamine, produces bradykinesia. Tremors in the hands, fingers, forearm, or foot tend to occur when the limb is at rest, but not when performing tasks. Tremor may occur in the mouth and chin as well. Rigidity, or stiff muscles, may produce muscle pain and an expressionless, mask-like face. Rigidity tends to increase during movement. Poor balance is due to the impairment or loss of the reflexes that adjust posture in order to maintain balance. Falls are common in people with Parkinson's. The Parkinsonian gait is the distinctive unsteady walk associated with Parkinson's Disease. There is a tendency to lean unnaturally backward or forward, and to develop a stooped, head-down, shoulders-drooped stance. Arm swing is diminished or absent and people with Parkinson's tend to take small shuffling steps (called Destination). Someone with Parkinson's may have trouble starting to walk, appear to be falling forward as they walk, freeze in mid-stride, and have difficulty making a turn.

Parkinson's Disease symptoms may also include, micrographia (small hand writing), resting tremor, freezing episodes, painful leg cramps, akinesia—difficulty initiating movement, muscle stiffness, difficulty getting up from a chair, stooped over posture, facial masking, hypomimia—loss of facial expression, hypophonia—low voice volume, monotone speech, slurred, soft speech, staring, reduced blinking, eyelid apraxia, small shuffling steps, poor balance, rigidity—muscle, cogwheel rigidity—stop/start movements, drooling, seborrhea—unusually oily skin, fatigue easily, reduced arm swing, reduced ability to perform tasks such as handflipping and finger tapping, constipation, difficulty swallowing (dysphagia)—saliva and food that collects in the mouth or back of the throat may cause choking, coughing, or drooling, excessive salivation (hypersalivation), excessive sweating (hyperhidrosis), loss of bladder and/or bowel control (incontinence), loss of intellectual capacity (dementia)—late in the disease, slow response to questions (brady phrenia) as well as psychosocial disorders such as, for example, anxiety, depression, and isolation.

There is no absolute diagnosis for Parkinson's Disease to date and there is great clinical need for developing a sensitive non-invasive diagnostic.

Diagnosis and monitoring of Parkinson's disease patients is critical for assessing severity of progression to respond with the appropriate preventative care. During the onset of Parkinson's disease, timely intervention could be life-saving. A comprehensive imaging modality for assessing Parkinson's disease remains a significant unmet clinical need.

In vivo measurements of dopamine activity are used for understanding how this key neuromodulator contributes to cognition, neurodevelopment, aging, and neuropsychiatric disease in humans. In medicine, such measurements can result in objective biomarkers that predict clinical outcomes, including Parkinson's disease, ideally by using procedures that capture the underlying pathophysiology while being easy to acquire in clinical settings.

Neuromelanin (“NM”) is a dark pigment synthesized via iron-dependent oxidation of cytosolic dopamine and subsequent relation with proteins and lipids in midbrain dopamine neurons. NM pigment accumulates inside specific autophagic organelles, which contain NM-iron complexes, along with lipids and various proteins. NM-containing organelles accumulate gradually over the lifespan in the soma of dopamine neurons in the substantia nigra (“SN”) a nucleus that owes its name to its dark appearance due to the high concentration of NM, and are only cleared from tissue following cell death through the action of microglia, such as through Parkinson's disease. Given that NM-iron complexes are paramagnetic, they can be imaged using MRI. A family of MRI sequences known as NM-MRI captures groups of neurons with high NM content, such as those in the SN, as hyperintense regions. NM-MRI signal is reliably decreased in the SN of patients who have Parkinson's disease consistent with the degeneration of NM-positive SN dopamine cells and with the decrease in NM concentration in post mortem SN tissue of Parkinson's patients compared to age-matched controls. While this evidence supports the use of NM-MRI for in vivo detection of SN neuron loss in neurodegenerative illness, direct demonstrations that this MRI procedure is sensitive to regional variability in NM concentration even in the absence of neurodegenerative SN pathology are lacking. Furthermore, although induction of dopamine synthesis via L-DOPA administration is known to induce NM accumulation in rodent SN cells and although prior work assumed that NM-MRI signal in the SN indexes dopamine neuron function in humans, direct evidence is lacking to support the assumption that inter-individual differences could lead to MRI-detectable differences in NM accumulation.

Thus, it would be beneficial to provide a system, process, method and computer-accessible medium for neuromelanin-sensitive MRI which can overcome the deficiencies described above.

### SUMMARY

Provided herein are, inter alia, methods for determining the presence of Parkinson's disease in a subject and determining the change in the concentration of neuromelanin in the subject with time. The concentration of neuromelanin may change as the result of the regular course of Parkinson's disease or as a result of therapeutic intervention. In a first aspect, there is provided a method of determining whether a change in the concentration of neuromelanin occurs over time in the brain of a subject. In a preferred embodiment, the subject is a Parkinson's disease patient. The method includes obtaining a first neuromelanin magnetic resonance image of the subject at a first time point. Subsequently a second neuromelanin magnetic resonance image is obtained at a second time point. The first magnetic resonance image is compared to the second magnetic resonance image, thereby determining whether a change in the concentration of neuromelanin occurred between the first time point and the second time point.

In one embodiment, the present invention is directed to a method of diagnosing, Parkinson's disease in a subject comprising:

(i) performing a Neuromelanin-Magnetic Resonance Imaging (NM-MRI) scan, measuring a level of neuromelanin,

(ii) comparing the level of neuromelanin to previous scans and/or reference values, and

(iii) providing a diagnosis of Parkinson's disease.

In one embodiment, the present invention is directed to a method of monitoring progression of Parkinson's disease in a subject comprising:

(i) performing a Neuromelanin-Magnetic Resonance Imaging (NM-MRI) scan, measuring a level of neuromelanin,

(ii) comparing the level of neuromelanin to previous scans and/or reference values, and

(iii) determining the progression of Parkinson's disease.

In one embodiment, the present invention is directed to a method of providing a prognosis of Parkinson's disease in a subject comprising:

(i) performing a Neuromelanin-Magnetic Resonance Imaging (NM-MRI) scan, measuring a level of neuromelanin,

(ii) comparing the level of neuromelanin to previous scans and/or reference values, and

(iii) optionally providing a prognosis of Parkinson's disease.

In one embodiment, the present invention is directed to a method of monitoring treatment of Parkinson's disease in a subject comprising:

(i) performing a Neuromelanin-Magnetic Resonance Imaging (NM-MRI) scan, measuring a level of neuromelanin,

(ii) comparing the level of neuromelanin to previous scans and/or reference values, and

(iii) assessing the effect of treatment of Parkinson's disease.

In one embodiment, the present invention is directed to determining a first signal intensity from a first neuromelanin magnetic resonance image and determining a second signal intensity from a second neuromelanin magnetic resonance image, and comparing the first magnetic resonance image to said second magnetic resonance image comprises comparing the first signal intensity to the second signal intensity.

In one embodiment, the control is a level of neuromelanin present at approximately the same levels in a population of subjects, or said standard control is approximately the average level of neuromelanin present in a population of subjects.

In one embodiment, a neuromelanin gradient phantom is used to measure the level, signal and/or concentration of neuromelanin.

In one embodiment, a neuromelanin phantom concentration gradient is scanned about once per patient, about once an hour, about once a day, about once a week, or about once a month.

In one embodiment, the neuromelanin phantom gradient is scanned daily.

In one embodiment, the neuromelanin phantom gradient is scanned with each patient.

In one embodiment, the present invention is directed to a method of assessing the neuromelanin in a subject comprising:

performing an Neuromelanin-Magnetic Resonance Imaging (NM-MRI) scan on the subject;

acquiring a neuromelanin dataset from the NM-MRI scan;

optionally encrypting the neuromelanin dataset;

uploading the neuromelanin dataset to a remote server;

optionally decrypting the dataset;

performing an analysis of the neuromelanin dataset, wherein the analysis comprises one or more of:
(i) comparing the neuromelanin dataset with one or more previously acquired neuromelanin datasets from the said subject
(ii) comparing the neuromelanin dataset with a control dataset;
(iii) comparing the neuromelanin dataset with one or more previously acquired neuromelanin datasets from different subjects;

generating a report comprising the neuromelanin analysis;

optionally encrypting the report;

uploading the report to remote server;

optionally decrypting the report.

In one embodiment, the invention is directed to an in vivo method of determining the progression of Parkinson's disease over time in a subject, said method comprising:

(i) obtaining a first neuromelanin magnetic resonance image at a first time point;

(ii) after step (i) comparing the first neuromelanin magnetic resonance image to an age matched control;

(iii) determining the level, signal and/or concentration of neuromelanin occurred between said first time point and said second time point.

In one embodiment, the invention is directed to an in vivo method of diagnosing Parkinson's disease, said method comprising:

(i) obtaining a first neuromelanin magnetic resonance image at a first time point;

(ii) after step (i), obtaining a second neuromelanin magnetic resonance image at a second time point;

(iii) comparing the first neuromelanin magnetic resonance image to said second neuromelanin magnetic resonance image thereby determining whether a change in one or more of the level, signal or concentration of neuromelanin occurred between said first time point and said second time point.

In one embodiment, the invention is directed to a method of providing a treatment regimen to a patient comprising performing the NM-MRI scan, acquiring NM signal from the NM-MRI scan in a region of interest, comparing the NM signal from the NM-MRI scan in a region of interest data to age matched database numbers, if the NM signal is less than a pre-determined value, administering a corresponding treatment regimen.

In one embodiment, the subject displays symptoms of Alzheimer's disease.

In one embodiment the patient suffers from a disorder commonly misdiagnosed as Parkinson's disease. In one embodiment this disorder is essential tremor. In one embodiment, this disorder is familial tremor.

In one embodiment, the NM-MRI scan and analysis distinguishes between Alzheimer's disease and Parkinson's disease. In one embodiment, the NM-MRI scan and analysis distinguishes between and can separately identify related disorders (e.g. MSA, PSP, Parkinsonism symptoms, dyskinesia, dystonia). In one embodiment, the NM-MRI scan and analysis can monitor the progression of, monitor the treatment of, and provide a prognosis for disorders related to Parkinson's disease (e.g. MSA, PSP, Parkinsonism symptoms, dyskinesia, dystonia).

In one embodiment, the present invention is directed to a method of determining if a subject has or is at risk of developing Parkinson's disease, the method comprising analyzing one or more Neuromelanin-Magnetic Resonance Imaging (NM-MRI) scans of the subject's brain region of interest, wherein the analyzing comprises:

receiving imaging information of the brain region of interest; and

determining a NM concentration in the brain region of interest using voxelwise analysis based on the imaging information;

wherein the determining if a subject has or is at risk of developing Parkinson's disease comprises:

(1) if the one or more NM-MRI scans has a decreased NM signal compared to a one or more control scans without Parkinson's disease then the subject has or is at risk of developing Parkinson's disease; or

(2) if the one or more NM-MRI scans has a NM signal comparable to the signal of a one or more control scans without Parkinson's disease then the subject does not have or is not at risk of developing Parkinson's disease.

In one embodiment, the present invention is directed to a method of treating a subject with Parkinson's disease, the method comprising analyzing Neuromelanin-Magnetic Resonance Imaging (NM-MRI) scans of the subject's brain region of interest, wherein the analyzing comprises:

receiving imaging information of the brain region of interest at a first time point;

receiving imaging information of the brain region of interest at a second time point;

determining a NM concentration at the first and second time points in the brain region of interest using voxelwise analysis based on the imaging information; and

comparing the NM concentration at the first time point to the second time point,

wherein the treatment method further comprises:

(1) administering one or more of levodopa and carbidopa if the NM-MRI scan at the second time point has a decreased NM signal compared to the NM signal at the first time point; or

(2) withhold administering one or more of levodopa and carbidopa if the NM-MRI scan at the second time point has an increased NM signal compared to the NM signal at the first time point.

In one embodiment, the subject exhibits one or more symptom of Parkinson's disease.

In one embodiment, the method provides a diagnosis of Parkinson's disease before symptoms present clinically.

In one embodiment, the NM-MRI method distinguishes between Alzheimer's disease and Parkinson's disease.

In one embodiment, the NM-MRI method diagnoses the patient as having Parkinson's disease or as not having Parkinson's disease; and indicates the diagnosis to a user via a user interface.

In one embodiment, the analysis is a voxelwise analysis.

In one embodiment, the voxelwise analysis comprises determining at least one topographical pattern within the brain region of interest.

In one embodiment, the method further comprises a calculation using a value that represents a volume of a neuromelanin voxel.

In one embodiment, the voxelwise analysis region of interest is the substantia nigra.

In one embodiment, the voxelwise analysis region of interest the ventral substantia nigra subregion.

In one embodiment, the invention is directed to a diagnostic system for providing diagnostic information for Parkinson's disease, the diagnostic system comprising:

an MRI system configured to generate and acquire a neuromelanin sensitive MRI scan along with a neuromelanin data series for a voxel located within a region of interest in a subject's brain;

a signal processor configured to process the series of neuromelanin data to produce a processed neuromelanin MRI spectrum; and

a diagnostic processor configured to process the processed neuromelanin MRI spectrum to:

extract a measurement from the region of interest corresponding with neuromelanin at a time point,

compare the measurement to one or more control measurements acquired prior to the time point;

provide a diagnosis of Parkinson's disease if the measurement is more than about 25% less than the control measurement.

In another aspect, there is provided a method for determining whether brain tissue in a subject contains an abnormal level of neuromelanin. The method includes detecting a level neuromelanin in the tissue. The level of neuromelanin is compared to a standard control. If a lower level of neuromelanin is detected relative to the standard control, this indicates Parkinson's disease.

In one embodiment, there is provided a method for determining whether a Parkinson's disease therapy administered to a subject is effective. The method includes a step of detecting a level of endogenous neuromelanin in the tissue at a first time point. In a subsequent step, a therapy is administered to the subject. A level of neuromelanin in the tissue is then determined at a second time point. Thereafter, the level of neuromelanin at the first time point is compared to the level of neuromelanin at the second time point. A higher level of neuromelanin at the second time point relative to the first time point indicates that the therapy was effective. Alternatively, a lower level neuromelanin at the second time point relative to the first time point indicates that the therapy administered to the subject was ineffective.

In one embodiment, there is provided a method for treating a patient with Parkinson's disease. In one embodiments, the method comprises administering to a patient an initial amount of L-dopa. In one embodiment, the method comprises monitoring the neuromelanin concentration in a region of interest in the patient's brain and assessing treatment-related adverse events over an initial treatment period. In one embodiment, if, during the initial treatment period, the patient exhibits one or more of
i) decreased neuromelanin concentration in the region of interest in the patient's brain; and
ii) no L-dopa associated adverse or side effects;
then increasing the dose of L-dopa in a subsequent treatment period;
wherein the L-dopa treatment results in an improvement in Parkinson's disease symptoms in the patient.

In one embodiment, the treatment method, includes the following step:

repeating steps a)-c) until the patient fails to exhibit one or more of i)-ii) in step c).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-B show MRI images. (A) Template of the midbrain in MNI space created by averaging spatially normalized NM-MRI images from all participants. The substantia nigra (SN) is clearly visible as a hyperintense region. (B) A mask of the SN (yellow, an over-inclusive mask to ensure full SN coverage for all participants) and the crus cerebri reference region (cyan) in MNI space was traced on the NM-MRI template and applied to all participants for calculation of contrast-to-noise ratio (Methods).

FIGS. 2A-D show comparisons between cocaine users and control. (A) Diagnostic group differences in NM-MRI signal between cocaine users and controls. Scatterplots showing extracted NM-MRI signal (CNR) averaged within cocaine-use voxels (top panel, defined in C), cocaine-use voxels as defined with leave-one-out (LOO) procedure (middle panel), and the whole SN (bottom panel) in participants divided based on diagnosis. To complement results showing the effect of diagnostic group on NM-MRI signal after adjusting for covariates (B and statistics reported in the text), these scatterplots show diagnostic group differences in the raw, unadjusted NM-MRI signal. (B) Receiver-operating-characteristic curves displaying sensitivity and specificity of the NM-MRI signal in separating diagnostic groups based on signal extracted from cocaine-use voxels (top panel), cocaine-use voxels defined with a leave one out procedure (middle panel), and whole SN (bottom panel). The black line represents NM-MRI signal adjusted for age, head coil, and tobacco use covariates; the gray line represents unadjusted NM-MRI signal. (B) Map of voxels where cocaine users exhibited higher NM-MRI signal than controls (shown in red, robust linear regression, p<0.05 one-sided). This set of voxels was above chance level (p_{corrected}=0.025, permutation test). (C) Unthresholded results of the same analysis showing the t-statistic for the diagnostic group effect for all SN voxels. Voxels where NM-MRI signal was higher in the cocaine users are shown in red and voxels where the signal was lower in cocaine users are shown in blue.

FIG. 3 shows a schematic depicting trafficking of dopamine between the cytosolic, vesicular, and synaptic pools in the striatum and subsequent accumulation of NM in the SN (curved arrow) in health and in cocaine use disorder. Boxes with dashed lines show a schematic detail of the striatal synapse between the gray, pre-synaptic dopamine neuron and the green, post-synaptic striatal neuron. Left: the cytosolic dopamine pool is normally converted to NM and accumulates gradually over the lifespan in the cell bodies of pre-synaptic dopamine neurons within the SN in the midbrain. Right: a theoretical scenario is presented to account for changes observed in cocaine use disorder including the decreased dopamine release observed with PET in prior literature and the increased NM-MRI signal reported here. A decrease in VMAT2, also consistent with PET and postmortem studies, could account for both of these: decreased VMAT2 expression would decrease vesicular dopamine and increase the cytosolic dopamine pool from which NM is synthesized. Please see text for alternative interpretations of the data.

FIG. 4 shows clinical and demographic measures.

FIG. 5 shows demographic and clinical characteristics for studies presented in Example 4.

FIGS. 6A-B show that baseline NM-MRI CNR correlates with gait speed at baseline. (a) Map of SN-VTA voxels where NM-MRI CNR positively correlated (thresholded at P<0.05, voxel level) with a single-task measure of gait speed (green voxels) overlaid on the average NM-MRI CNR image from all subjects. (b) Scatterplot showing the average NM-MRI CNR extracted from the significant voxels in a plotted against gait speed for visualization purposes. These plotted data show a Pearson correlation coefficient of 0.49, although this effect-size estimate is likely inflated given the selection of significant voxels for this effect.

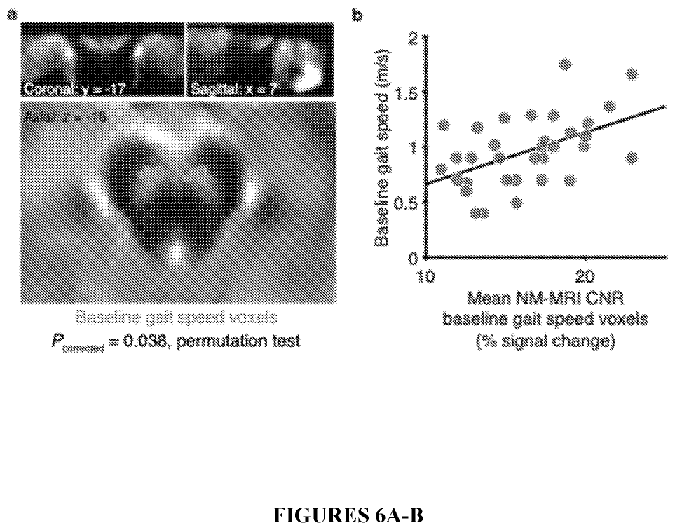

FIGS. 7A-B show that secondary analyses of baseline NM-MRI CNR do not predict changes in gait speed after 3 weeks of L-DOPA treatment in region-of-interest or voxelwise analyses. (a) Scatterplot showing the average NM-MRI CNR extracted from the significant (green) voxels in FIG. 1a plotted against gait speed. These plotted data have a Pearson correlation coefficient of 0.10. (b) Scatterplot showing the average NM-MRI CNR extracted from the voxels where NM-MRI CNR positively correlated with the change in gait speed after 3 weeks of L-DOPA treatment (N=64; thresholded at P<0.05, voxel level). These plotted data have a Pearson correlation coefficient of 0.17.

FIG. 8A-C show that NM-MRI CNR significantly increases after 3 weeks of L-DOPA treatment. (a) Map of SN-VTA voxels where NM-MRI CNR significantly increased after 3 weeks of L-DOPA (thresholded at P<0.05, voxel level; red voxels) overlaid on the average NM-MRI CNR image from all subjects. (b) Histogram showing the average change across subjects in NM-MRI CNR after treatment including all SN-VTA voxels, which is generally shifted to the right of zero (denoting increased NM-MRI CNR). For visualization purposes, heights are proportional to either the number of L-DOPA voxels (N=200; red bars corresponding to voxels in a or the number of Other SN-VTA Voxels (i.e., non-significant voxels; N=1607); e.g., a bar with voxel proportion of 0.2 for L-DOPA voxels corresponds to 40 voxels while a bar with voxel proportion of 0.2 for Other SN-VTA voxels corresponds to 321 voxels. (c) Ladder plot showing the average NM-MRI CNR extracted from the significant (red) voxels in a at baseline (Pre L-DOPA) and after 3 weeks of L-DOPA treatment (Post L-DOPA) for the 6 subjects (each shown in a different color to emphasize consistent increases across each subject).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### Definitions

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description is taken with the drawings making apparent to those skilled in the art how the forms of the present invention may be embodied in practice.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the disclosure of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range. Moreover, the terminology at least includes the stated number, e.g., “at least 50” includes 50.

The term “MR” refers to magnetic resonance and is the physical principle upon which a variety of experimental procedures known in the art and/or described herein are based, including MRI (“magnetic resonance imaging”), MRS (“magnetic resonance spectroscopy”) and the like. The term neuromelanin-sensitive MRI or neuromelanin-MRI refer to the use of MRI in the study of neuromelanin in the brain. Herein the general term magnetic resonance image, magnetic resonance imaging or MRI encompasses neuromelanin-sensitive variants.

As used herein, the term “NM-MRI” and similar nomenclature refers to each the MRI scan and corresponding voxel wise analysis independently, both as separate and together.

The terms “T1” and “T2” used herein refer to the conventional meanings well known in the art (i.e., “spin-lattice relaxation time,” and “spin-spin relaxation time,” respectively).

The term “T1-weighted” in the context of MRI images refers to an image made with pulse spin echo or inversion recovery sequence, having appropriately shortened TR and TE, which as known in the art can demonstrate contrast between tissues having different T1 values. The term “TR” in this context refers to the repetition time between excitation pulses. The term “excitation pulse” is understood to refer to a 90-deg radio frequency (RF) excitation pulse. The term “TE” refers to the echo time between the excitation pulse and MR signal sampling.

The term “subject” may be a mammalian subjects such as murine, Rattus, equine, bovine, ovine, canine, feline or human. In some embodiments of the methods described herein, the subject is a mouse, while in other embodiments the subject is a human. The term “patient” in this context refers to a human subject.

As used herein, the term “alleviate” is meant to describe a process by which the severity of a sign or symptom of a disorder is decreased. Importantly, a sign or symptom can be alleviated without being eliminated. In a preferred embodiment, the use of treatment methods disclosed herein leads to the elimination of a sign or symptom, however, elimination is not required. Effective dosages guided by the present invention are expected to decrease the severity of a sign or symptom.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. An effective amount of a pharmaceutical agent is that which provides an objectively identifiable improvement.

As used herein, “stable” refers to measurements that are reproducible. In one embodiment, “stable neuromelanin levels” refers to serial scans where neuromelanin levels remain relatively constant. In some contexts, “stable neuromelanin levels” are maintained for one or more hours, one or more days, one or more weeks, or one or more treatment cycles.

The terms “treat,” “treatment” and the like in the context of disease refer to ameliorating, suppressing, eradicating, and/or delaying the onset of the disease being treated. In some embodiments, the methods described herein are conducted with subjects in need of treatment. The terms “in need of treatment” and the like as used herein refer to a subject at risk for developing a disease, having a condition, which would be understood by those of skill in the medical or veterinary arts as likely leading to a disease, and/or actually having a disease. Parkinson's disease treatments includes currently approved and investigative treatments. The NM-MRI of the present invention can monitor the efficacy of Parkinson's treatment. The NM-MRI of the present invention can determine efficacy of investigative treatments. A non-exhaustive listing of Parkinson's treatment which may be monitored according to one embodiment of the present invention includes one or more of the following:

Parkinson's disease treatments include disease-modifying therapies. These therapies aim to prevent, slow or halt the overall progression of Parkinson's disease (PD). They target different proteins and pathways believed to play a role in the disease.

Alpha-synuclein; This protein forms toxic clumps in some brain and body cells of people with PD.

Anle138b; MODAG's small molecule aims to inhibit aggregation of alpha-synuclein. MJFF funded pre-clinical work and a portion of a Phase I trial in people with Parkinson's.

BIIB054; Biogen's antibody aims to prevent aggregated alpha-synuclein from spreading. MJFF is funding tool development and data collection that support study design.

NPT088; Proclara's (previously Neurophage) drug candidate aims to prevent alpha-synuclein from clumping together. MJFF funded pre-clinical work.

PD01A; AFFiRiS' vaccine aims to stimulate antibodies against alpha-synuclein. MJFF funded pre-clinical work, a portion of the Phase I trial and boost studies.

RO7046015; Prothena/Roche's antibody aims to prevent aggregated alpha-synuclein from spreading. MJFF is funding tool development and data collection that support study design.

GBA; Mutations in the GBA gene are associated with Parkinson's disease and are linked to certain cellular dysfunction.

GZ/SAR402671; Sanofi Genzyme's drug reduces production of lipids that build up with GBA mutations. MJFF is funding tool development and data collection that support study design.

LTI-291; Lysosomal Therapeutics' oral drug may offset dysfunction associated with GBA mutation. MJFF funded the pre-clinical work.

LRRK2; Mutations in the LRRK2 gene are associated with Parkinson's disease and linked to greater activity of the LRRK2 protein.

DNL201; Denali's LRRK2 inhibitor aims to lower heightened LRRK2 activity. MJFF funded safety studies that supported the trial.

Exenatide; Diabetes medication that has protected brain cells in pre-clinical Parkinson's models. MJFF funded the Phase II trial led by the University of College London.

Inosine; Nutritional supplement raises urate (antioxidant) levels. Population studies show inosine may have a protective effect or slow progression of PD. MJFF funded pre-clinical work and the Phase II trial and is supporting biomarker collection in the Phase III trial led by the Parkinson Study Group.

Isradipine; High blood pressure drug may help protect brain cells.

Nilotinib; This treatment for a cancer of the white blood cells (chronic myelogenous leukemia) may address dysfunction seen in PD.

Parkinson's disease treatments include neurotrophic factors. Trophic factors are like the brain's natural fertilizer; they help restore and protect neurons. GDNF; MedGenesis' trophic factor may protect dopamine cells. MJFF funded the pre-clinical work. CDNF; Herantis' trophic factor may protect dopamine cells. MJFF funded the pre-clinical work.

Parkinson's disease treatments include those that improve motor symptoms. Tremor, stiffness and slowness of movement affect mobility. Levodopa can help, but it does not treat all symptoms, can feel less effective with time and may bring side effects such as dyskinesia with long-term use.

Levodopa Delivery; The gold standard for motor symptom treatments can, with long-term use, wear off and cause side effects, such as dyskinesia. Researchers believe some side effects may be due to fluctuating levels of levodopa. Accordion Pill; Layers of levodopa/carbidopa release slowly from the stomach for better absorption. NDO612; Neuroderm's levodopa/carbidopa pump or pump-patch could maintain steady levels of levodopa.

Levodopa is a prodrug of dopamine that is administered to patients with Parkinson's due to its ability to cross the blood-brain barrier. Levodopa can be metabolised to dopamine on either side of the blood-brain barrier and so it is generally administered with a dopa decarboxylase inhibitor like carbidopa to prevent metabolism until after it has crossed the blood-brain barrier. Once past the blood-brain barrier, levodopa is metabolized to dopamine and supplements the low endogenous levels of dopamine to treat symptoms of Parkinson's. The first developed drug product that was approved by the FDA was a levodopa and carbidopa combined product called Sinemet.

Parkinson's disease treatments include non-dopamine approaches. Targeting other brain chemicals with add-on therapies may help control motor fluctuations associated with levodopa use. PXT002331; Prexton Therapeutics' oral drug (foliglurax) works on the glutamate and other brain chemical systems to reduce motor symptoms and dyskinesia.

Parkinson's disease treatments include “Off” Rescue therapeutics. When levodopa levels diminish, patients' symptoms can return; this is called an “off” episode. APL-130277; Sunovion's (previously Cynapsus) thin film of the drug apomorphine placed under the tongue could rescue patients from “off” episodes. CVT-301; Acorda's (previously Civitas) inhaled levodopa can quickly ease symptoms.

Parkinson's disease treatments include gene therapy. With surgery, a selected gene is delivered to the brain to increase production of deficient protein. AAV2-hAADC; Voyager's approach aims to replace the enzyme AADC in brain cells to improve levodopa conversion to dopamine for better control of motor symptoms and less “off” time in advanced Parkinson's.

Conventional MRI lacks the spatial and quantitative data needed to predict clinical outcomes in neurotrauma. However, the methods as discussed herein detect levels of neuromelanin in the brain that can predict clinical progression, severity, and response in Parkinson's disease given the variance of neuromelanin in the brain or loss of neuromelanin-containing neurons.

In some embodiments NM-MRI provides a method for dose titration for the treatment of Parkinson's disease while avoiding and adverse or side effects from currently approved or investigational therapeutics. Specifically, administering L-dopa while monitoring NM signals using the voxelwise approach described herein to guide the dosage regimen, it is possible to increase efficacy compared with L-dopa administration alone

Additionally, administering a therapeutic according to a specific variable dosage regimen guided by NM-MRI, it is possible to reduce side effects which may be associated with administration. For example, administering L-dopa according to the specific dosage regimen guided by NM-MRI voxel analysis of the present invention may significantly reduce, or even completely eliminate treatment associated side effects.

In certain embodiments, doses of L-dopa are increased, reduced, administered more frequently or administered less frequently depending on physiological factors, including but not limited to neuromelanin increases or decreases in a region of interest in the subject brain either compared to previous scans or compared to baseline control. In one embodiment, the region of interest are Parkinson's disease symptom-associated voxels. The dose variation increases patient compliance, improves therapy and reduces unwanted and/or adverse effects. In certain embodiments, the therapeutic method of the invention provides an improved overall therapy relative to the administration of the therapeutic agents by themselves.

In certain embodiments, doses of existing agents can be reduced or administered less frequently in using the guided intervention of the present invention, thereby increasing patient compliance, improving therapy and reducing unwanted or adverse effects. In one embodiment, monitoring treatment with the NM-MRI of the present invention allows patients to experience benefit from treatment for a longer timeframe.

Neuromelanin-sensitive MRI data may be used as a biomarker for Parkinson's disease, or risk of developing Parkinson's disease, severity, illness progression, treatment response, and/or clinical outcome. Neuromelanin-sensitive MRI methods meet the need for objective biomarker tracking Parkinson's disease, severity, or risk for its development. Neuromelanin-sensitive MRI can be used as a safe alternative for invasive/radiating imaging measures (e.g., PET). Neuromelanin-sensitive MRI can also be used for monitoring of progression, which currently cannot be done given the risk of repeated exposure to radiation. Neuromelanin-sensitive MRI is non-invasive, cheaper, safer, and easier to acquire in clinical settings. It has substantially increased (5-10-fold) anatomical resolution, which allows for resolving anatomical detail within relevant brain structures.

In certain embodiments, neuromelanin sensitive magnetic resonance images are obtained periodically, for example, every 1, 2, 3, 4, 5, 6 or 7 days, every 1, 2, 3 or 4 weeks, every 1, 2, 3, 4, 5, 6, 7, 8, 9, 10, 11 or 12 months, or every 1, 2, 3, 4 or 5 years. In certain embodiments, a first magnetic resonance image is obtained prior to the appearance of symptoms. In certain embodiments, a first magnetic resonance image is obtained prior to symptoms associated with Parkinson's disease. A second magnetic resonance image may be obtained either prior to or subsequent to the appearance of symptoms. In other embodiments, a second magnetic resonance image may be obtained 1 year after the first magnetic resonance image.

In some embodiments, the neuromelanin sensitive magnetic resonance imaging (“NM-MRI”) technique is effective at non-invasively diagnosing, measuring the effect of, and/or providing a prognosis for Parkinson's disease.

In some embodiments, the NM-MRI technique is used as a tool for diagnosing presymptomatic Parkinson's disease. In some embodiments, the NM-MRI technique is effective for distinguishing Parkinson's disease from other neurological conditions, including but not limited to Alzheimer's disease. In other embodiments, the NM-MRI technique is effective at selecting a course of treatment, optionally, such a treatment is effective at treating Parkinson's disease.

In some embodiments, the NM-MRI technique is used as a tool for monitoring the progress of Parkinson's disease. In some embodiments, the NM-MRI technique is effective for the longitudinal assessment of Parkinson's disease progression.

In some embodiments the technique measures neuromelanin directly or indirectly. In other embodiments, the technique measures dopamine function directly or indirectly. In some embodiments, there is a connection between neuromelanin-sensitive MRI (NM-MRI) signal and Parkinson's disease severity.

In some embodiments, the NM-MRI technique is capable of determining the concentrations of neuromelanin across all sections of brain tissue. In other embodiments, the NM-MRI technique is capable of determining regional concentrations of neuromelanin. In other embodiments, the NM-MRI technique is capable of determining regional levels of neuromelanin. In other embodiments, the NM-MRI technique is capable of determining regional signal intensity of neuromelanin.

In other embodiments, the NM-MRI technique determines the neuromelanin concentration in the substantia nigra subregions. In further embodiments, the NM-MRI technique determines dopamine release in the dorsal striatum and resting blood flow within the substantia nigra either directly or indirectly.

In some embodiments, the NM-MRI signal and Parkinson's disease severity are directly correlated. In some embodiments, the NM-MRI signal and Parkinson's disease severity are inversely correlated. In other embodiments, NM-MRI exhibits lower signal in the nigrostriatal pathway of people with Parkinson's disease. In some embodiments, the NM-MRI captures dopamine dysfunction. In yet other embodiments, the NM-MRI can be used as a biomarker for Parkinson's disease. In further embodiments, the NM-MRI can be used to determine the severity of Parkinson's disease. In further embodiments, the NM-MRI can be used to diagnose and/or provide a prognosis for Parkinson's disease.

In some embodiments, the analysis is performed in comparison to previous NM-MRI. In other embodiments, the analysis is performed in comparison to a reference value and/or range. In some embodiments, the reference value and/or range is generated using a compilation of neuromelanin data from healthy people. In some embodiments, the reference value and/or range is generated using a compilation of neuromelanin data from people who have Parkinson's disease. In some embodiments, the reference value and/or range is generated using a compilation of neuromelanin data from people who have Parkinson's disease and people who do not have Parkinson's disease.

In some embodiments, the NM-MRI signal is taken from the lateral substantia nigra. In other embodiments, the NM-MRI signal is taken from the posterior substantia nigra. In further embodiments, the NM-MRI signal is taken from the ventral areas of the substantia nigra. In some embodiments, the NM-MRI signal is taken from one or more of the lateral, posterior and ventral areas of the substantia nigra.

In some embodiments, the NM-MRI signal is taken from the substantia nigra or the locus coeruleus. In some embodiments, the NM-MRI signal is taken from the ventral substantia nigra. In some embodiments, the NM-MRI signal is taken from the lateral substantia nigra. In some embodiments, the NM-MRI signal is taken from the ventrolateral substantia nigra. In some embodiments, the NM-MRI signal is taken from the substantia nigra pars Compacta (SNpc). In some embodiments, the NM-MRI signal is taken from the substantia nigra pars reticulata (SNpr). In some embodiments, the NM-MRI signal is taken from the ventral tegmental area (VTA). In some embodiments, the NM-MRI signal is taken from the locus coeruleus. In some embodiments, the NM-MRI signal is taken from one or more of the ventral substantia nigra lateral substantia nigra, ventrolateral substantia nigra substantia nigra pars Compacta (SNpc), substantia nigra pars reticulata (SNpr), ventral tegmental area (VTA), and the locus coeruleus.

In some embodiments, the NM-MRI technique comprises assessing the neuromelanin in a subject comprising: perform an MRI scan; acquire neuromelanin data; optionally encrypt neuromelanin data; optionally upload neuromelanin data to a remote server; optionally decrypt data; perform analysis of neuromelanin data, wherein the analysis optionally comprises comparing neuromelanin data against previously acquired data, a large population database, or both; generate a report comprising neuromelanin analysis; optionally encrypt the report; optionally upload the report to remote server; optionally decrypt the report.

In some embodiments, the report provides a diagnosis for Parkinson's disease. In some embodiments, a physician or imaging technician decrypts the report. In further embodiments, the analysis is performed remotely. In other embodiments, the remote analysis is performed on a cloud platform. In other embodiments, the remote analysis is performed on a cloud server.

In one embodiment, the invention is directed to analyzing and classifying Parkinson's disease in a subject, such as a human subject of research or study, or a patient, for example. The subject data is acquired by a NM-MRI measurement. A plurality of templates classified according to degree of Parkinson's disease are stored in a data store, such as a database, for example. Each of the templates represents selected subsets of neuromelanin measures among populations measured from at least one other subject known to have Parkinson's disease. The set of data is processed to obtain a model that represents temporal measures among neuromelanin concentration in a subject. A comparison is made of at least a portion of the neuromelanin data with the plurality of templates to produce a classification of Parkinson's disease.

Embodiments of the invention include diagnostic tools for use in clinical settings, or tools for evaluating subjects in research settings. More generally, aspects of the invention provide tools for obtaining an assessment or diagnosis of Parkinson's disease utilizing NM-MRI. Systems and methods according to various aspects of the invention are useful for monitoring a potentially changing condition of a subject, such as a progression of Parkinson's disease, for example. Additionally, aspects of the invention provide solutions for monitoring treatment effectiveness of patients.

A second aspect of the invention is a method of screening for therapeutic agents that prevent, delay or halt the development or progression of Parkinson's disease or corresponding symptoms in a patient, comprising: 1) exposing said patient to at least one candidate therapeutic agent; 2) measuring the neuromelanin concentration, and 3) assessing the effect of the at least one therapeutic agent on the development or progression of Parkinson's disease or corresponding symptoms in the patient.

Certain embodiments of the present invention can provide an objective test to enhance diagnostic accuracy, advance the recognition of Parkinson's disease into a presymptomatic stage, and serve as a monitor for therapy. In general, embodiments of the present invention can be used to diagnose neuromelanin using a stored template, differentiate between a number of different conditions or diseases, and monitor a subject over a period of time.

In one embodiment, the invention is used with a second imaging method, wherein the second imaging method is positron emission tomography (PET). In one embodiment, the invention is used with a second imaging method, wherein the second imaging method is structural MRI. In one embodiment, the invention is used with a second imaging method, wherein the second imaging method is functional MRI (fMRI). In one embodiment, the invention is used with a second imaging method, wherein the second imaging method is blood oxygen level dependent (BOLD) fMRI. In one embodiment, the invention is used with a second imaging method, wherein the second imaging method is iron sensitive MRI. In one embodiment, the invention is used with a second imaging method, wherein the second imaging method is quantitative susceptibility mapping (QSM). In one embodiment, the invention is used with a second imaging method, wherein the second imaging method is diffusion tensor imaging DTI. In one embodiment, the invention is used with a second imaging method, wherein the second imaging method is single photon emission computed tomography (SPECT). In one embodiment, the invention is used with a second imaging method, wherein the second imaging method is DaTscan. In one embodiment, the invention is used with a second imaging method, wherein the second imaging method is DaTquant.

In some embodiments, the neuromelanin concentration and/or level is measured against a control and if the neuromelanin concentration and/or level is about 5%, about 10%, about 15%, about 20%, about 25%, about 30%, about 35%, about 40%, about 50%, about 60%, about 70%, about 80%, about 90% less than the control a diagnosis of Parkinson's disease is supported. In some embodiments, the change in neuromelanin is assessed as a net concentration or level change per year. In some embodiments, the change in neuromelanin is assessed as a percentage change per year. In some embodiments, the neuromelanin concentration and/or level is measured against a control and the neuromelanin concentration and/or level is about 1%, about 2%, about 3%, about 4%, about 5%, about 6%, about 7%, about 8%, about 9%, about 10%, about 11%, about 12%, about 13%, about 14%, or about 15% less than the control. In some embodiments, the neuromelanin concentration and/or level is measured against a control and the neuromelanin concentration and/or level is about 1%, about 2%, about 3%, about 4%, about 5%, about 6%, about 7%, about 8%, about 9%, about 10%, about 11%, about 12%, about 13%, about 14%, or about 15% decreased per year compared to the control. In one embodiment, the control is a patient's prior NM-MRI scan and voxelwise analysis. In one embodiment, neuromelanin concentration and/or level is measured against a control and the neuromelanin concentration and/or level is measured yearly, every 2 years, every 3 years, every 4 years, every 5 years, every 6 years, every 7 years, every 8 years, every 9 years, every 10 years, every 20 years. In one embodiment, the second time point is about 3 months, about 6 months, about 9 months, about 12 months, about 2 years, about 3 years, about 4 years, about 5 years, about 6 years, about 7 years, about 8 years, about 9 years, about 10 years, about 15 years, about 20 years, about 25 years, or about 30 years after the first time period. In certain embodiments, when the neuromelanin concentration and/or level is measured to be less than the control, a patient is diagnosed with Parkinson's disease. In certain embodiments, when the neuromelanin concentration and/or level is measured to be a pre-determined amount less than the control either per year or net overall change, a patient is diagnosed with Parkinson's disease. In further embodiments, the measured neuromelanin is more than about 20% less than the control. In further embodiments, the measured neuromelanin is more than about 25% less than the control. In further embodiments, the measured neuromelanin is more than about 30% less than the control. In further embodiments, the measured neuromelanin is more than about 35% less than the control. In further embodiments, the measured neuromelanin is more than about 45% less than the control. In further embodiments, the measured neuromelanin is more than about 40% less than the control. In further embodiments, the measured neuromelanin is more than about 50% less than the control. In certain embodiments, the control is optionally a previous neuromelanin MRI scan of the same patient. In other embodiments, the control comprises a reference number optionally determined from a database of neuromelanin MRI scans from at least one other person with Parkinson's disease.

In one embodiment, if the change in the level, signal and/or concentration of neuromelanin at the second time point is more than about 5% less or more than about 10% less than the level, signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Parkinson's disease is provided.

In one embodiment, if the change in the level, signal and/or concentration of neuromelanin at the second time point is more than about 35% less, more than about 40% less, more than about 45% less, or more than about 50% less signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Parkinson's disease is provided.

In one embodiment, the degree of reduction in neuromelanin volume, signal, or concentration in a given patient compared to a control is proportional to the progression and/or severity of Parkinson's disease.

In one embodiment, the degree of increase in neuromelanin volume, signal, or concentration in a given patient compared to a control is proportional to the improvement and/or efficacy of Parkinson's disease progression and/or treatment.

In one embodiment, the standard control is a level of neuromelanin present at approximately the same levels in a population of subjects, or the standard control is approximately the average level of neuromelanin present in a population of subjects.

To illustrate extending the use of NM-MRI for such applications, a series of validation studies is shown. A first procedure is provided to show that NM-MRI can be sensitive enough to detect regional variability in tissue concentration of NM, which presumably depends on inter-individual and inter-regional differences in dopamine function (e.g., including synthesis and storage capacity), and not just to loss of NM-containing neurons. To test this, MRI measurements were compared to neurochemical measurements of NM concentration in post-mortem tissue without Parkinson's disease. Because variability in dopamine function may not occur uniformly throughout all SN tiers, the next procedure was to show that NM-MRI, which has high anatomical resolution compared to standard molecular-imaging procedures, has sufficient anatomical specificity. NM-MRI is used to test the ability of a novel voxelwise approach to capture the known topographical pattern of cell loss within the SN in Parkinson's disease. The next procedure is then to provide direct evidence for a relationship between NM-MRI and Parkinson's disease using the voxelwise approach.

As discussed in WO 2020/077098, incorporated herein in its entirety by reference, NM-MRI signal correlates to a well-validated Positron Emission Tomography (“PET”) measure of dopamine release into the striatum—the main projection site of SN neurons—and to a functional MRI measure of regional blood flow in the SN, an indirect measure of activity in SN neurons, in a group of individuals without Parkinson's disease. Level of neuromelanin increases (SNc concentration, volume of NM in SNc), as measured by Terran NM-101, that results in improvement in UPDRS with L-Dopa therapy

The present invention correlates Parkinson's voxels with Parkinson's symptoms as measured by UPDRS; demonstrates that the application of the voxel-based analysis method in Terran NM-101 will find specific voxels (termed PD voxels) unique to each patient that correlate with their specific symptoms on UPDRS; determines the correlation between the change in neuromelanin measures after initiation of L-DOPA therapy and improvement in UPDRS scores; determines the differences in neuromelanin measures (e.g. total NM concentration (microgram neuromelanin per microgram wet tissue) in substantia nigra pars Compacta (SNc), NM concentration in the subregions SNc, volume of neuromelanin in the total SNc, volume of subregions of the SNc) in a patient with PD from the normal range of the control group; determines the difference in neuromelanin levels from a control group that would warrant a diagnosis of PD; correlates the change in neuromelanin measures after initiation of L-DOPA therapy and improvement in UPDRS scores; determines the level of neuromelanin increase that results in improvement in UPDRS to validate that NM levels can be used to monitor response to treatment; correlates Parkinson's voxels with Parkinson's symptoms measured via UPDRS scores; applies the voxel based analysis method to find specific voxels (termed PD voxels) unique to each patient that correlate with their specific symptoms on UPDRS; correlation between NM-MRI scans and both DaTscan and UPDRS scores.

In one embodiment, L-dopa is a representative treatment for any Parkinson's disease treatment. In one embodiment, L-dopa represents Carbidopa/Levodopa. In one embodiment, the treatment is gene therapy. In one embodiment, if the neuromelanin concentration remains stable, unchanged or constant, the dosage of L-dopa remains constant. In one embodiment, if the neuromelanin concentration remains stable, the dosage of L-dopa is increased. In one embodiment, if the neuromelanin concentration is decreased by more than about 1%, more than about 2%, more than about 3%, more than about 5%, more than about 10%, more than about 15%, more than about 20%, or more than about 25%, then the L-dopa dose is increased. In one embodiment, the neuromelanin is monitored by serial scans. In one embodiment, the neuromelanin is measured according to symptom-specific voxels in a single patient. In one embodiment, the symptom specific voxels are specific to Parkinson's disease. In one embodiment, the Parkinson's specific voxels are determined in a patient by comparing a patient's NM-MRI data to pre-determined set of controls from other patients. In one embodiment, the set of controls from other patients is age matched. In one embodiment, the set of controls from other patients is gender matched.

In some embodiments, the neuromelanin is measured at least every other day, every week, every 2 weeks, every month, every other month, every 3 months, every 6 months, every year, every 2 years, every 3 years, every 4 years, every 5 years, every 6 years, every 7 years, every 8 years, every 9 years, every 10 years, every 15 years, every 20 years, every 25 years, every 30 years In certain embodiments, the second therapeutic agent dose is administered every week or every 2 weeks. In certain embodiments, the therapeutic is administered, every 1 hour, 2 hours, 3 hours, 4 hours, 5 hours, 6 hours, 8 hours, 10 hours, 12 hours, 14 hours, 16 hours, 18 hours, 20 hours, 24 hours, 1 day, 2 days, 3 days, 4 days, 5 days, 6 days, 7 days, or at least 14 days.

In one embodiment, the treatment period (either initial or subsequent) or monitoring period as discussed herein is every day, every other day, every 28 days, every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks, about every month, about every other month, about every 3 months, about every 6 months or about every year.

In one embodiment, a region of interest is determined and voxels that cover that region are measured to determine the volume of neuromelanin in that area.

In one embodiment, the region of interest is subdivided and voxels that cover the subregions are measured to determine the volume of neuromelanin in that area.

In one embodiment, these voxels are compared to a reference dataset and used to compute the concentration of neuromelanin in the region of interest or subregions within the region of interest.

In one embodiment, these voxels are compared to a reference dataset and used to compute the total amount of neuromelanin in the region of interest or subregions within the region of interest.

In one embodiment, multiple comparisons are performed between all of the voxels identified in the region of interest and specific symptoms or scales of symptom severity, or disease states, or demographic information, or other patient or disease specific information, and associations are found between a subgroup of individual voxels and a specific symptom or level of symptom severity on a disease monitoring scale. These are termed symptom-specific voxels.

In one embodiment, multiple comparisons are performed between all of the voxels identified in the region of interest and specific disease diagnoses or demographic information, or other patient or disease specific information, and associations are found between a subgroup of individual voxels and the condition of being diagnosed with a specific disease. These are termed disease-specific voxels and in one example may comprise Parkinson's-disease-specific voxels.

In one embodiment, these symptom-specific or disease-specific voxels have similarities across multiple patients with the same symptom in the context of the same disease and can be used to make comparisons between multiple patients with the same disease (for example two patients with Parkinson's disease who both have the symptom of psychomotor slowing). In this case the similarities between patients may be compared and the symptom specific voxels may function as a diagnostic biomarker.

In one embodiment, these symptom-specific or disease-specific voxels have differences between patients with the same symptom occurring in the context of different diseases. In this case differences between the symptom specific voxels can be used to differentiated between two different disorders sharing the same symptom.

In one embodiment, either symptom-specific voxels or disease-specific voxels, or neuromelanin concentrations, or neuromelanin volumes of specific regions or subregions can be used as a non-invasive biomarker to determine diagnostic information, to diagnose the presence of a specific disease (in this case Parkinson's disease or a related disorder such as MSA, PSP, Parkinsonism symptoms, dyskinesia, dystonia, or essential tremor.

In one embodiment, this can be accomplished by comparing the baseline measurements in a specific patient of either symptom-specific voxels or disease-specific voxels, or neuromelanin concentrations, or neuromelanin volumes of specific regions or subregions against future measurements of these values in the same patient.

In one embodiment, this can be accomplished by comparing the measurements in a specific patient of either symptom-specific voxels or disease-specific voxels, or neuromelanin concentrations, or neuromelanin volumes of specific regions or subregions against a standard control.

In one embodiment, either symptom-specific voxels or disease-specific voxels or neuromelanin concentrations, or neuromelanin volumes of specific regions or subregions can be used as a non-invasive biomarker to determine diagnostic information, to rule-out the presence of a related disorder or differentiate between related disorders such as Parkinson's disease and MSA, PSP, Parkinsonism symptoms, dyskinesia, dystonia, or essential tremor.

In one embodiment, this can be accomplished by comparing the baseline measurements in a specific patient of either symptom-specific voxels or disease-specific voxels, or neuromelanin concentrations, or neuromelanin volumes of specific regions or subregions against future measurements of these values in the same patient.

In one embodiment, this can be accomplished by comparing the measurements in a specific patient of either symptom-specific voxels or disease-specific voxels, or neuromelanin concentrations, or neuromelanin volumes of specific regions or subregions against a standard control.

In one embodiment, either symptom-specific voxels or disease-specific voxels or neuromelanin concentrations, or neuromelanin volumes of specific regions or subregions can be used as a non-invasive biomarker to stage or grade a specific disease or symptom and differentiate or classify this information in a patient. For example, this may be used to determine the stage of PD or a related motor disorder in a specific patient

In one embodiment, either symptom-specific voxels or disease-specific voxels or neuromelanin concentrations, or neuromelanin volumes of specific regions or subregions can be used as a non-invasive biomarker to determine the current severity of symptoms in a patient.

In one embodiment, either symptom-specific voxels or disease-specific voxels or neuromelanin concentrations, or neuromelanin volumes of specific regions or subregions can be used as a non-invasive biomarker to predict the development of new symptoms that the patient has not yet developed.

In one embodiment, either symptom-specific voxels or disease-specific voxels or neuromelanin concentrations, or neuromelanin volumes of specific regions or subregions can be used as a non-invasive biomarker to predict the severity of current symptoms, predict the future development of a disease course, or predict the response of either a specific symptom or the response of the disease as a whole response to treatment and function as a non-invasive prognostic biomarker.

In one embodiment, either symptom-specific voxels or disease-specific voxels or neuromelanin concentrations, or neuromelanin volumes of specific regions or subregions can be used as a non-invasive biomarker to monitor response to treatment for either a specific symptom or a disease state as a whole.

In one embodiment, either symptom-specific voxels or disease-specific voxels or neuromelanin concentrations, or neuromelanin volumes of specific regions or subregions can be used as a non-invasive biomarker to guide the selection of the correct treatment for either a specific symptom or a disease state as a whole.

In one embodiment, either symptom-specific voxels or disease-specific voxels or neuromelanin concentrations, or neuromelanin volumes of specific regions or subregions can be used as a non-invasive biomarker to determine the status of treatment and determine if an adequate response to treatment has been obtained for either a specific symptom or a disease state as a whole.

In one embodiment, either symptom-specific voxels or disease-specific voxels or neuromelanin concentrations, or neuromelanin volumes of specific regions or subregions can be used as a non-invasive biomarker to predict the future response to treatment for either a specific symptom or a disease state as a whole.

In any embodiment, comparisons may be made between:

The baseline measurements in a specific patient of either symptom-specific voxels or disease-specific voxels, or neuromelanin concentrations, or neuromelanin volumes of specific regions or subregions against future measurements of these values in the same patient.

the measurements in a specific patient of either symptom-specific voxels or disease-specific voxels, or neuromelanin concentrations, or neuromelanin volumes of specific regions or subregions against a standard control.

### Example 1-Validation Experiments of NM-MRI Voxel Wise Analysis Exemplary Relationship to Nm Concentration in Post-Mortem Midbrain Tissue

A test was performed to determine whether NM-MRI can be sensitive to variation in NM tissue concentration at levels found in individuals without major neurodegeneration of the SN, a prerequisite for its use as a marker of inter-individual variability in healthy and populations suffering from Parkinson's disease. To this end, this is validated against gold-standard measures of NM concentration by scanning SN-containing midbrain sections from individuals without histopathology compatible with Parkinson's disease or Parkinson's disease-related syndromes using a NM-MRI sequence. After scanning, each specimen is dissected along gridline markings into 13-20 grid sections. In each grid section, the tissue concentration of NM is measured using biochemical separation and spectrophotometry determination and also calculated the averaged NM-MRI contrast-to-noise ratio (“CNR”) across voxels within the grid section.

Across all midbrain specimens, grid sections with higher NM-MRI CNR had higher tissue concentration of NM (β₁=0.56, t₁₁₄=3.36, p=0.001, mixed-effects model; 116 grid sections, 7 specimens. As expected, hyperintensities were most apparent in grid sections corresponding with the NM-rich SN. But, similar to in vivo NM-MRI images posterior-medial regions of the midbrain around the periaqueductal gray (“PAG”) area tended to appear hyperintense despite relatively low concentrations of NM. Although the source of this hyperintensity, controlling for the presence of PAG in grid sections (e.g., PAG) improved the correspondence of NM-MRI CNR to NM concentration in non-PAG regions (131=1.03, 1112=5.51, p=10⁻⁷) could not be accounted for. Under this model, a 10% increase in NM-MRI CNR corresponds with an estimated increase of 0.10 μg of NM per mg of tissue.

The relationship between NM-MRI CNR and NM concentration remained (β₁=0.45, t₁₁₁=2.15, p=0.034) in an extended model controlling for the proportion of SN voxels within each grid section (e.g., and again for PAG content). This latter result suggests that NM-MRI CNR explains variance in NM concentration in the SN and surrounding areas beyond that explained simply by the increase in both measures in SN compared to non-SN voxels, an increase that would be expected even if NM-MRI could only localize the SN without measuring regional NM concentration. These results thus indicate that NM-MRI signal corresponds to regional tissue concentration of NM, particularly in the midbrain region surrounding the SN, the region focused on.

Exemplary Validation of Voxel Wise Approach

Having shown that NM-MRI measures regional concentration of NM in and around the SN, whether regional differences in NM-MRI signal capture biologically meaningful variation across anatomical subregions within the SN was determined. This was needed to use this tool to interrogate dopamine function, since the heterogeneity of cell populations in the SN suggests that dopamine function can differ substantially between neuronal tiers projecting to ventral striatum, dorsal striatum or cortical sites. It was determined that a voxel wise analysis within the SN can be sensitive to processes affecting specific subregions or likely discontiguous neuronal tiers within the SN for information regarding spatial normalization and anatomical masks used in voxel wise analyses). Supporting the feasibility of this approach, the majority of individual SN voxels exhibited good-to-excellent test-retest reliability, extending similar demonstrations at the region level.

To test the anatomical specificity of the voxel wise NM-MRI approach, the ability of NM-MRI to detect disease and the known topography of cell loss in the illness is utilized.

Using NM-MRI data in 28 patients and 12 age-matched controls, whether a voxel wise analysis would capture this topographic pattern is analyzed. The exemplary approach is able to capture the known anatomical topography of dopamine neuron loss within the SN: larger CNR decreases tended to predominate in more lateral (β_{|x|}=−0.13, t₁₈₀₃=−14.2, p=10⁻⁴³) posterior (β_{y}=−0.05, t₁₈₀₃=−6.6, p=10⁻¹⁰), and ventral SN voxels (β_{z}−0.17, t₁₈₀₃=16.3, p=10⁻⁵⁵; multiple-linear regression analysis predicting t statistic of group difference across SN voxels as a function of their coordinates in x [absolute distance from the midline], y, and z directions: omnibus F_{3,1803}=111, p=10⁻⁶⁵)

Exemplary Relationship of NM-MRI Signal to Dopamine Function

Having validated the anatomical sensitivity of the exemplary voxel wise approach, whether NM-MRI signal in the SN correlated with dopamine function in vivo is analyzed. To that end, positron emission tomography (“PET”) imaging is used to measure dopamine release capacity (ΔBP_{ND}) as the change in D2/D3 radiotracer [¹¹C]raclopride binding potential between baseline and following administration of dextro-amphetamine (0.5 mg/kg, p.o.). This method measures the release of dopamine from the presynaptic sites of dopamine axons, including its vesicular and cytosolic pools into striatal synapses, so it can be relevant that trait-like inter-individual differences in the magnitude of these dopamine pools can be a key determinant of NM accumulation. Data in a group of 18 individuals without neurodegenerative illness is collected, which includes 9 healthy controls and 9 unmedicated patients with neurodegeneration. Dopamine release in the associative striatum, part of the dorsal striatum, was focused on to ensure sufficient variability, as patients with Parkinson's disease tend to exhibit the differences in dopamine release in this subregion. Also, the dorsal striatum receives projections from the SN (e.g., via the nigrostriatal pathway) while the ventral striatum receives projections predominantly from the ventral tegmental area (e.g., via the mesolimbic pathway), which can be more difficult to visualize in NM-MRI scans due to its lower NM concentration and smaller size. A voxel wise analysis was performed in which, for each subject, ΔBP_{ND} was measured and correlated to NM-MRI CNR in the SN mask at each voxel. This revealed a set of SN voxels where NM-MRI CNR correlated positively with dopamine release capacity in the associative striatum (e.g., 225 of 1341 SN voxels at p<0.05, Spearman partial correlation adjusting for diagnosis, age, and head coil; p_{corrected}=0.042, permutation test; peak voxel MNI coordinates [x, y, z]: −1, −18, −16 mm). This effect exhibited a topographic distribution such that voxels related to dopamine release tended to predominate in anterior and lateral aspects of the SN. This analysis was performed in a smaller SN mask (e.g., 1341 voxels) because relatively few subjects had usable data in the dorsal-most SN. No interaction with diagnosis was found (e.g., p=0.31). The voxel wise results were mirrored by region-of-interest (“ROI”) results showing that mean NM-MRI CNR across the whole SN correlated with mean ΔBP_{ND} in the whole striatum (e.g., p=0.64, p=0.013; partial correlation with the same covariates as the voxel wise analysis and an additional covariate for incomplete SN coverage.

Exemplary Relationship of NM-MRI Signal to Neural Activity in the SN

Since the latter results indicated that individuals with higher dopamine release from nigrostriatal SN neurons had higher NM accumulation, as measured via NM-MRI, it was determined that NM accumulation can also correlate with local trait-like tendency for increased activity in SN neurons. To test this, arterial spin labeling functional magnetic resonance imaging (“ASL-fMRI”) was used to measure regional cerebral blood flow (“CBF”), a well-established (e.g., indirect) functional measure of neuronal activity that captures trait-like inter-individual differences in resting activity. Individuals without neurodegenerative illness (e.g., 12 healthy individuals, 19 schizophrenia patients) higher CBF in the SN correlated with higher SN NM-MRI CNR was found. This was true in ROI analyses averaging values in SN voxels related to dopamine release capacity (e.g., “dopamine voxels”, r=0.40, p=0.030; partial correlation controlling for age and diagnosis) and in the whole SN (e.g., r=0.48, p=0.008; partial correlations controlling for age, diagnosis, and incomplete SN coverage). Again, no interactions with diagnosis were found (e.g., all p>0.7).

Relationship of NM-MRI to Parkinson's Disease

NM-MRI as a measure of NM concentration in the SN, can be used as a marker of neuronal loss in those who have Parkinson's disease or suffer from a symptom indicative of Parkinson's disease.

Showing that NM-MRI can capture the changes in neuromelanin concentrations associated with Parkinson's disease supports the potential value of NM-MRI as a research tool and neuromelanin concentration as a candidate biomarker for Parkinson's disease. This phenomenon has been identified in patients with history of Parkinson's disease—in some embodiments, the phenomenon is in proportion to the severity of their experiences. In certain embodiments, the Parkinson's disease is characterized by one or more symptoms. The exemplary procedure suggests that this Parkinson's disease-related phenotype consisting of nigrostriatal dopamine excess results in a decrease in NM accumulation in the SN that can be captured with NM-MRI. Specifically, a mostly ventral SN subregion was found where NM-MRI CNR can be decreased in proportion to severity of Parkinson's disease. The exemplary findings further underscore the promise of NM-MRI as a clinically useful biomarker for conditions associated with neuromelanin concentration. It has the obvious advantages of being practical (e.g., inexpensive and non-invasive), particularly for longitudinal imaging, and of providing high anatomical resolution compared to standard imaging methods, which facilitates it to resolve functionally distinct SN tiers with different pathophysiological roles. The presumed ability of NM-MRI to index NM long-term, given the slow accumulation of NM in the SN over the lifespan and the high reproducibility of this procedure suggest that NM-MRI can be a stable marker insensitive to acute states (e.g., recent sleep loss or substance consumption). This can be a particularly appealing characteristic for a candidate biomarker and one that could complement other markers. A dimensional marker of Parkinson's disease-related NM changes would be extremely helpful as a longitudinal biomarker for Parkinson's disease. Such a biomarker could further help select a subset of at-risk individuals who, more so than CHR individuals as a whole can benefit from medication, thus augmenting current risk-prediction procedures based solely on non-biological measures.

There can be some limits to the potential applications of NM-MRI. Similar to other neuroimaging measures, the exemplary data show that the NM-MRI signal can be sensitive but not fully specific to NM concentration. Other tissue properties, including proton density, can impact the signal. Thus, caution in interpreting all changes in NM-MRI signal as changes in NM concentration can be warranted, especially in regions with low NM concentration.

Exemplary Longitudinal Monitoring of Parkinson's Disease

MR images are acquired about every year, about every two years, about every three years, about every four years, about every five years and the neuromelanin level, signal and/or concentration is measured. The neuromelanin level, signal and/or concentration is compared to previous scans. After comparing the level, signal and/or concentration of neuromelanin decreasing with respect to time indicates progression of the condition. In some embodiments, the decrease is proportional to the progression or the severity of Parkinson's disease. In some embodiments, a medicine is administered after the first MRI scan and an MRI scan at a second time point after the administration of the medicine. Comparing the two scans can indicate success in the treatment regimen.

Exemplary NM-MRI Acquisition

MR images were acquired for all study participants on a GE Healthcare 3T MR750 scanner using a 32-channel, phased-array Nova head coil. For logistical reasons, a few scans (e.g., 17% of all scans, 24 out of a total of 139) were acquired using an 8-channel in vivo head coil instead. During piloting various NM-MRI sequences were compared to achieve optimal CNR in the SN using a 2D gradient response echo sequence with magnetization transfer contrast (e.g., 2D GRE-MT) with parameters: repetition time (TR)=260 ms, echo time (TE)=2.68 ms, flip angle=40°, in-plane resolution=0.39×0.39 mm², partial brain coverage with field of view (FoV)=162×200, matrix=416×512, number of slices=10, slice thickness=3 mm, slice gap=0 mm, magnetization transfer frequency offset=1200 Hz, number of excitations [NEX]=8, acquisition time=8.04 minutes. The slice-prescription protocol consisted of orienting the image stack along the anterior-commissure-posterior-commissure (“ACPC”) line and placing the top slice 3 mm below the floor of the third ventricle, viewed on a sagittal plane in the middle of the brain. This protocol provided coverage of SN-containing portions of the midbrain (e.g., and cortical and subcortical structures surrounding the brainstem) with high in-plane spatial resolution using a short scan easy to tolerate by clinical populations. Whole-brain, high-resolution structural MRI scans were also acquired for pre-processing of the 2D GRE-MT (e.g., NM-MRI) data: a T1-weighted 3D BRAVO sequence (e.g., inversion time=450 ms, TR≈7.85 ms, TE≈3.10 ms, flip angle=12°, FoV=240×240, matrix=300×300, number of slices=220, isotropic voxel size=0.8 mm³) and a T2-weighted CUBE sequence (e.g., TR=2.50 ms, TE≈ms, echo train length=120, FoV=256×256, number of slices=1, isotropic voxel size=8 mm³). Quality of NM-MRI images was visually inspected for artifacts immediately upon acquisition and scans were repeated when necessary, time permitting. Ten participants were excluded due to clearly visible, smearing or banding artifacts affecting the midbrain (e.g., due to participant motion, n=4), or incorrect imaging-stack placement (e.g., n=6).

Exemplary NM-MRI Preprocessing

NM-MRI scans were preprocessed using SPM12 to facilitate voxel wise analyses in standardized MNI space. For example, NM-MRI scans and T2-weighted scans were coregistered to T1-weighted scans. Tissue segmentation was performed using T1- and T2-weighted scans as separate channels. Scans from all study participants were normalized into MNI space using DARTEL routines with a gray- and white-matter template generated from an initial sample of individuals. The resampled voxel size of unsmoothed, normalized NM-MRI scans was 1 mm, isotropic. All images were visually inspected following each preprocessing procedure. Intensity normalization and spatial smoothing were then performed using custom Matlab scripts. CNR for each subject and voxel v was calculated as the relative change in NM-MRI signal intensity I from a reference region RR of white-matter tracts known to have minimal NM content, the crus cerebri, as CNR_{V}=(I_{V}−mode(I_{RR}))/mode(I_{RR}). A template mask of the reference region in MNI space was created by manual tracing on a template NM-MRI image (e.g., an average of normalized NM-MRI scans from the initial sample individuals). The mode(I_{RR}) was calculated for each participant from kernel-smoothing-function fit of a histogram of all voxels in the mask. The mode rather than mean or median was utilized because it was found it to be more robust to outlier voxels (e.g., due to edge artifacts) and this precluded the need for further modification of the reference-region mask. Images were then spatially smoothed with a 1-mm full-width-at-half-maximum Gaussian kernel.

Further, an over inclusive mask of SN voxels was created by manual tracing on the template NM-MRI image. The mask was subsequently reduced by eliminating edge voxels with extreme values: voxels showing extreme relative values for a given participant (e.g., beyond the 1^{st} or the 99^{th} percentile of the CNR distribution across SN voxels in more than 2 subjects) or voxels that had consistently low signal across participants (e.g., CNR less than 5% in more than 90% of subjects). These procedures removed 9% of the voxels in the manually traced mask, leaving a final template SN mask containing 1,807 resampled voxels.

Exemplary NM-MRI Analysis

All analyses were carried out in Matlab (Mathworks, Natick, Mass.) using custom scripts. In general, robust linear regression analyses were performed across subjects for every voxel v within the SN mask, as: CNR_{V}=β₀+β₁ measure of interest++Σᵢ₌₂ⁿβᵢ nuisance covariate+ε. The measure of interest consisted of either imaging (e.g., dopamine release capacity) or clinical (e.g., Parkinson's disease severity) data, depending on the analysis. Nuisance covariates, including diagnosis, head coil, and age, varied for different analyses; while all analyses included an age covariate, head coil and diagnosis covariates were only included in analyses where these variables differed across subjects. Robust linear regression was used to minimize the need for regression diagnostics in the context of mass-univariate, voxel wise analyses. A partial (e.g., non-parametric) Spearman correlation was used instead of linear regression if variables were not normally distributed according to a Lilliefors test at p<0.05 (e.g., which was the case for dopamine release capacity). Voxel wise analyses were carried out within the template SN mask after censoring subject data points with missing values (e.g., due to incomplete coverage of the dorsal SN in a minority of subjects resulting from inter-individual variability in anatomy) or extreme values (e.g., values more extreme than the 1st or the 99^{th} percentile of the CNR distribution across all SN voxels and subjects [CNR values below −9% or above 40%, respectively]). For all voxel wise analyses, the spatial extent of an effect was defined as the number of voxels k (e.g., adjacent or non-adjacent) exhibiting a significant relationship between the measure of interest and CNR (e.g., voxel-level height threshold for t-test of regression coefficient β₁ of p<0.05, one-sided [β₁⁺)].

Hypothesis testing was based on a permutation test in which the measure of interest was randomly shuffled with respect to CNR. This test corrected for multiple comparisons by determining whether an effect's spatial extent k was greater than would be expected by chance (e.g., p_{corrected}<0.05, 10,000 permutations; equivalent to a cluster-level family-wise-error-corrected p-value, although in this case voxels were not required to form a cluster of adjacent voxels, given the small size of the SN and evidence that SN tiers defined by specific projection sites do not necessarily comprise anatomically clustered neurons). On each iteration, the order of the values of a variable of interest (e.g. dopamine release capacity) was randomly permuted across subjects (e.g., and maintained for the analysis of every voxel within the SN mask for a given iteration of the permutation test, accounting for spatial dependencies). This provided a measure of spatial extent for each of 10,000 permuted datasets, forming a null distribution against which to calculate the probability of observing the spatial extent k of the effect in the true data by chance (p_{corrected}). For hypothesis testing related to conjunction effects, permutation analysis determined if the extent k of overlap for both effects (β⁺_{1effect1}∩β⁺_{1effect2}) was greater than would be expected by chance (e.g., p<0.05, 10,000 permutations) based on a null distribution counting the overlap of significant voxels after the location of true significant voxels for each effect was randomly shuffled within the SN mask.

Exemplary Topographical analyses. Multiple-linear regression analysis across SN voxels was used to predict the strength of an effect (e.g., or the presence of a significant conjunction effect) as a function of MNI voxel coordinates in the x (e.g., absolute distance from the midline), y, and z directions.

Exemplary ROI analyses. Post hoc ROI analyses examining mean NM-MRI signal across voxels in the whole SN mask included the same covariates as used in the respective voxelwise analyses plus an additional dummy covariate indexing subjects with incomplete coverage of dorsal SN, as a dorsal-ventral gradient of signal intensity in SN biased mean CNR values in these subjects. This “incomplete SN coverage” covariate was not used for analyses on NM-MRI signal extracted from “dopamine” voxels or “Parkinson's disease-overlap” voxels as these confined sets of voxels had a relatively small contribution from dorsal SN.

Exemplary Post-Mortem Experiment

Post-mortem specimens of human midbrain tissue were obtained from The New York Brain Bank at Columbia University. Seven specimens were obtained, each from an individual who suffered from Parkinson's disease. Specimens were ˜3-mm-thick slices of fresh frozen tissue from the rostral hemi-midbrain containing pigmented SN. These specimens were scanned using the NM-MRI protocol similar to the one used in vivo, after which they were dissected for analyses of NM tissue concentration. The dish containing the specimen included a grid insert, used to keep dissections in register with MR images.

Exemplary Neurochemical measurement of NM concentration in post-mortem tissue. Samples deriving from each grid section were homogenized with titanium tools. NM concentration of each grid section was then measured according to the exemplary previously described spectrophotometry method, with minor modifications to improve the removal of interfering tissue components from midbrain regions with higher content of fibers and fewer NM-containing neurons compared to sections of SN proper dissected along anatomical boundaries. Additional tests confirmed that the exemplary methods for Fomblin® cleaning were effective and that neither this substance nor the methylene blue dye was likely to influence spectrophotometric measurements of NM.

Exemplary MRI measurement of NM signal in post-mortem tissue. NM-MRI signal was measured in corresponding grid sections using a custom Matlab script. Processing of NM-MRI images included automated removal of voxels showing edge artifacts and signal dropout, averaging over slices to create a 2D image, and registration with a grid of dimensions matching the grid insert. The grid registration was adjusted manually based on the well markers and grid-shaped edge artifacts present in the superior-most slice where the grid insert rested. Signal in the remaining voxels was averaged within each grid section. To normalize signal intensity across specimens, CNR for each grid section was calculated as in the in vivo voxelwise. The reference region for each specimen was defined by the 3 grid sections that best matched the location of the crus cerebri reference region used for in vivo scanning.

Exemplary Statistical analysis of post-mortem data. A generalized linear mixed-effects (“GLME”) model including data across all grid sections g and specimens s was used to predict NM tissue concentration in each grid section based on mean NM-MRI CNR in the same grid section. GLME analyses used an isotropic covariance matrix and were fitted via maximum pseudo-likelihood estimation, as implemented via the Matlab function fitglme. Likelihood-ratio tests at p<0.05 favored reduced models without random slopes. Therefore, all models included random intercepts but not random slopes, as: [NM]_{gs}=β₀+β₁·C̅N̅R̅^̅{̅g̅s̅}̅+Σᵢ₌₂ⁿ βᵢ·nuisance covariate_{gs}+b_{Os}+ε_{gs}. The basic model only included mean NM-MRI CNR in a given grid section C̅N̅R̅^̅{̅g̅s̅}̅as a fixed-effects predictor. Sections near the PAG tended to have relatively high signal intensity but low NM tissue concentration. Thus, an extended model included a binary variable for PAG presence in grid sections (e.g., PAG+, PAG−) and an interaction term of NM-MR1 CNR×PAG as additional fixed-effects covariates (e.g., the interaction was significant at p=0.040, confirming that NM-MRI was less strongly related to NM concentration in PAG+ relative to PAG− regions). PAG+ grid sections (e.g., 1 to 5 per specimen) were defined as those situated at the posterior-medial aspect of the specimen and consistent with the anatomical location of the PAG. Finally, a control analysis additionally included a fixed-effects covariate indicating the proportion of voxels containing SN for each grid section, defined as the proportion of voxels with CNR higher than 10% in grid sections deemed to contain SN upon visual inspection. This latter control analysis aimed to test whether regional variability in NM-MRI CNR would predict regional variability in NM tissue concentration even after accounting for changes in both measures as a mere function of the presence or absence of SN neurons in a given region (e.g., in combination with partial-volume effects).

Exemplary PET Imaging Study

Subjects (e.g., healthy controls, Parkinson's disease patients) underwent PET scanning using the radiotracer [¹¹C]raclopride and an amphetamine challenge to quantify dopamine release capacity. A baseline PET scan was conducted on one day and a post-amphetamine PET scan was acquired the next day, 5-7 hours after administration of dextroamphetamine (e.g., 0.5 mg/kg, p.o.). For each PET scan, list-mode data were acquired on a Biograph mCT PET-CT scanner (Siemens/CTI, Knoxville Tenn.) over 60 minutes following a single bolus injection of [¹¹C]raclopride, binned into a sequence of frames of increasing duration and reconstructed by filtered back projection using manufacturer-provided software. PET data were motion-corrected and registered to the individuals' T1-weighted MRI scan using SPM2. ROIs were drawn on each subject's T1-weighted MRI scan and transferred to the coregistered PET data. Time-activity curves were formed as the mean activity in each ROI in each frame. The exemplary a priori ROI was the associative striatum, defined as the entire caudate nucleus and the precommissural putamen, a part of the dorsal striatum that receives nigrostriatal axonal projections from SN neurons and that has been consistently implicated in conditions associated with Parkinson's disease. Data were analyzed using the simplified reference-tissue model (“SRTM”) with cerebellum as a reference tissue to determine the binding potential relative to the non-displaceable compartment (e.g., BP_{ND}). The primary outcome measure was the relative reduction in BP_{ND} (ΔBP_{ND}), reflecting amphetamine-induced dopamine release, a measure of dopamine release capacity. Amphetamine induces synaptic release of dopamine derived from both cytosolic and vesicular stores. This results in excessive competition with the radiotracer at the D2 receptor, and, simultaneously, agonist-induced D2-receptor internalization, both of which cause radiotracer displacement and lower BP_{ND}. ΔBP_{ND} thus combines both effects and reflects the magnitude of dopamine stores. Since these stores depend on dopamine synthesis, the dopamine release capacity PET measure can be relevant to dopamine function. It can also be relevant to NM given that NM accumulation can be driven by cytosolic dopamine (e.g., or by vesicular dopamine once it can be transported into the cytosol).

Exemplary Arterial Spin Labeling (“ASL”) Perfusion Imaging Study

Subjects (e.g., healthy controls, Parkinson's disease patients) underwent ASL functional MRI scanning at rest to quantify regional CBF. All of these subjects also participated in the above study and are described below. Pseudo-continuous ASL (e.g., 3D-pCASL) perfusion imaging was performed using a 3D background suppressed fast spin-echo stack-of-spiral readout module with eight in-plane spiral interleaves (e.g., TR=4463 ms, TE=10.2 ms, labeling duration=1500 ms, post-labeling delay=2500 ms, no flow-crushing gradients, FoV=240×240, NEX=3, slice thickness=4 mm) and an echo train length of 23 to obtain 23 consecutive axial slices. A labeling plane of 10-mm thick was placed 20 mm inferior to the lower edge of the cerebellum. Total scan time was 259 s. The ASL perfusion data were analyzed to create CBF images using Functool software (version 9.4, GE Medical Systems). CBF was calculated as in prior work.

For preprocessing, CBF images were coregistered to ASL-localizer images, which were then coregistered to T1 images, with the coregistration parameters applied to CBF images. CBF images were then normalized into MNI space using the same procedures described above for NM-MRI scans. Mean CBF was calculated within the whole SN mask and within the mask of SN voxels significantly related to dopamine release capacity in the associative stratium. ROI-based partial correlation analyses tested the relationship between mean CBF and mean NM-MRI CNR in the same mask, controlling for age and diagnosis.

Further Post-Mortem Experiment

Post-mortem specimens of human midbrain tissue were obtained from The New York Brain Bank at Columbia University. Seven specimens were obtained, each from an individual who suffered from Alzheimer's disease or other non-PD dementia at the time of death (e.g., ages 44 to 90; for further clinical and demographic information see Table 1 below). None suffered from Parkinson's disease, Parkinson's disease symptoms, or any other movement disorder or neurodegenerative illness affecting the SN, based on neuropathological examination for accumulation of abnormal proteins such as alpha-synuclein, beta-amyloid or tau. One case showed marked decrease in neuronal density in the SN despite clearly identifiable NM. Analyses excluding this one case did not change the observed relationship between NM-MRI CNR and NM concentration. Therefore, the data presented include this case to increase statistical power. Specimens were s ˜3-mm-thick slices of fresh frozen tissue from the rostral hemi-midbrain of the right hemisphere containing pigmented SN. They were stored at −80° C. These specimens were scanned using the NM-MRI protocol, after which they were dissected for analyses of NM tissue concentration. For the MRI scanning session, the specimens were progressively thawed to 20° C., as verified via a laser thermometer. Specimens were placed in a custom-made dish 3D-printed from MRI-compatible nylon polymer (NW Rapid Mfg., McMinnville, Oreg.) and a matching grid-insert lid was placed on top of the specimen and affixed to hold the specimen in place. While secured in the dish, specimens were fully immersed in an MRI-invisible lubricant (Fomblin® perfluoropolyether Y25; Solvay, Thorofare, N.J.) and placed in a desiccator for 30 minutes to remove air from the tissue. Wells in the four cardinal points of the rim of the dish were filled with water to mark its location and orientation in the MRI images. The dishes were then placed on a custom stand inside a 32-channel, phased-array Nova head coil and scanned using the 2D GRE-MT NM-MRI sequence described above for in vivo imaging. The only changes in the post-mortem scanning protocol were an increase in the resolution (e.g., in-plane resolution=0.3125×0.3125 mm², slice thickness-0.60 mm) and a decrease in the FoV (e.g., 160×80).

After the scanning session, samples were refrozen in place and marked with gridlines by applying methylene blue dye (e.g., 0.05% water solution [5 mg/10 ml]; Sigma-Aldrich, St. Louis, Mo.) to the tissue using the grid insert as a stamp. Guides built into the walls of the dish ensured that the orientation of the grid with respect to the specimen was fixed at all times. Within 4 days post-scanning, partially thawed specimens were dissected along gridlines after extensive removal of Fomblin® by dripping tissue slices, followed by gently rolling the surface of sections on ultraclean filter paper. Dissection and manipulation of tissue sections were performed with ceramic blades and titanium-and-plastic forceps to avoid contamination from iron. Each grid section (e.g., 3.5 mm×3.5 mm×˜3 mm, depending on the slice thickness), together with any adjacent partial grid sections, was weighed, stored separately in Eppendorf tubes, and frozen. Specimens were thus divided into 13-20 grid sections; the grid column and row number of each dissected grid section was coded.

Exemplary NM-MRI Analysis: Exclusion of Voxels with Few Observations

To reduce the risk of type II error, voxels are excluded from the analysis if, after censoring of subject data points with missing or extreme values, the t-test of the regression coefficient β₁ for a particular analysis had fewer than 10 degrees of freedom (e.g., note that the degrees of freedom take into account the sample size with usable data in a given voxel as well as the number of model predictors). This voxel exclusion only applied to the analysis relating NM-MRI signal to dopamine release capacity given the smaller sample size of the PET dataset, and this analysis was thus performed for 1,341 resampled SN voxels (e.g., rather than for the full mask of 1,807 resampled voxels). Selecting exclusion thresholds anywhere between 8-11 degrees of freedom gave very similar results. See inset in for distribution of degrees of freedom for all voxels in this analysis.

Exemplary NM-MRI Analysis: Non-Circular Voxel Selection for Estimation of Unbiased Effect Size

For voxel wise analyses, an unbiased measure of effect size was generated by using a leave-one-out procedure: for a given subject, voxels where the variable of interest was related to NM-MRI signal were first identified in an analysis including all subjects except for this (e.g., held-out) subject. The mean signal in the held-out subject was then calculated from this set of voxels. This procedure was repeated for all subjects so that each subject had an extracted, mean NM-MRI signal value obtained from an analysis that excluded them. This unbiased voxel selection and data extraction thus avoided statistical circularity. Unbiased estimates of effect size (e.g., Cohen's d or correlation coefficient) were then determined by relating these extracted NM-MRI signal values to variables of interest across held-out subjects and including the same covariates as in the voxel wise analysis and an additional covariate indexing subjects lacking full dorsal-SN coverage (e.g., due to dorsal-ventral gradient in NM-MRI signal intensity).

Exemplary Neurochemical Measurement of NM Concentration in Post-Mortem Tissue: Examination of Chemical Agents Applied to Post-Mortem Tissue

To test whether Fomblin® influenced NM measurement, small cubes of SN pars Compacta with similar levels of pigmentation were dissected from a single healthy subject. Some cubes (e.g., n=3) were immersed in Fomblin®, then cleaned of the Fomblin® (e.g., drained and rolled on filter paper); the remaining cubes (e.g., n=5) were not immersed in Fomblin® as control samples. NM concentration was comparable in these two sets of cubes (e.g., mean+standard deviation: 0.82+0.08 versus 0.86±0.09 μg NM/mg wet tissue, respectively; t₆=−0.62, p=0.56). The water-soluble methylene blue dye was efficiently removed during washing procedures in the exemplary standard protocol to measure NM concentration; moreover, it was confirmed that the absorption wavelength of this compound (e.g., with a peak near 680 nm) can be far from that used in the determination of NM concentration (e.g., 350 nm).

Exemplary MRI Measurement of NM Signal in Post-Mortem Tissue: Automated Removal of Voxels Showing Edge Artifacts and Signal Dropout

Processing of NM-MRI images included automated removal of low-signal voxels, including all voxels outside of the specimen or voxels within the specimen showing signal dropout. The threshold for exclusion of low-signal voxels was determined for each specimen based on the histogram of all voxels in the image, which was fitted using a kernel smoothing function. The threshold was defined as the signal corresponding to the minimum lying between the leftmost peak in the fitted histogram, corresponding to low-signal voxels outside of the specimen, and the rightmost peak, corresponding to higher-signal voxels within the specimen (e.g., consistent with a bimodal distribution).

To eliminate edge artifacts, the first procedure was to define the boundaries between the specimen and the surrounding space outside the specimen and between the specimen and areas of signal dropout. These boundaries were defined in 3D and 2D. To do so, boundary voxels of the specimen that lay directly next to low signal voxels, (defined above, were labeled using the bwperim function in Matlab these boundary voxels were defined for the whole volume and also for a 2D flattened image created by averaging over slices. These boundary voxels were removed from the specimen (e.g., first the 3D border voxels were removed from the 3D image, then the 2D boundary voxels, dilated by 2 voxels, were removed from the resulting flattened image). Finally, voxels with extreme signal values (e.g., Cook's distance>4/n in a constant-only linear regression model) relative to other voxels in the same 2D grid section were removed. The resultant 2D image, cleaned of edge artifacts, signal dropout and other outlier voxels, was carried forward to the final analysis procedures.

Exemplary PET Imaging Study: Timing of Post-Amphetamine PET Scan

Each subject received 2 post-amphetamine PET scans for the purposes of a separate experiment, which was previously published. This previous study aimed at assessing the time course of receptor internalization after an agonist challenge, measured via prolonged displacement of the D2 radiotracer [¹¹C]raclopride. PET scans were acquired in four sessions: baseline, 3 h after amphetamine, 5 to 7 h after amphetamine and 10 h after amphetamine. However, not all time points post-amphetamine were available for all subjects. Displacement was however highly stable and did not differ between the 3-h and the 5-7-h time points (ΔBP_{ND} indeed strongly correlated across subjects between these two time points; r=0.75). Only one of these post-amphetamine scans was used: the one administered 5-7 hours post-amphetamine. The 5-7-h time point was selected, because this was the time point with the most available data (e.g., missing for only 3/18 participants for whom it was substituted by data from the 3 hour time point). Displacement at 5 to 7 hours post-amphetamine-like displacement at 3 hours post-amphetamine-reflects the magnitude of dopamine release due to amphetamine, which can be a combination of competition between dopamine and the radiotracer for binding to the receptor, and agonist-induced receptor internalization, both of which depend on the magnitude of agonist availability. Thus, the 5-7-h time point can be the optimal time point for this study due to the larger number of subjects with available data and given the observed stability of the displacement between the 3-h and the 5-7-h time points. At the 10-h time point BP_{ND}) tended to be higher, likely due to a decrease in receptor internalization following recycling of receptors. Examining the 11 subjects with PET data at 3 hours revealed that the effect size of the correlation between NM-MRI CNR and ΔBP_{ND} at this 3 hour time point was similar to that at the 5-7 hour time point.

Exemplary Study Related to Parkinson's Disease Using Neuromelanin (NM) MRI

### BACKGROUND

Parkinson's Disease (PD) is a progressive motor neurodegenerative disorder that is the second most common neurodegenerative disorder after Alzheimer's disease among the elderly. PD, with the typical symptoms of resting tremor, bradykinesia, rigidity and postural instability, is defined primarily as a movement disorder and is pathologically characterized by degeneration of nigrostriatal dopaminergic neurons and the presence of Lewy bodies (misfolded α-synuclein) in the surviving neurons. The non-motor manifestations may include depression, autonomic dysfunction, cataracts and cognitive impairment, such as mild cognitive impairment and Parkinson's dementia.

Neuromelanin (NM) MRI signal is reliably decreased in the SN of patients with PD, consistent with the degeneration of NM-positive SN dopamine cells and with the decrease in NM concentration in postmortem SN tissue of PD patients compared with age-matched controls. Since NM-MRI was first utilized in 2002, there have been at least 35 clinical trials of NM changes in the substantia nigra have shown that it is a biomarker for degeneration in Parkinson's disease with high sensitivity and specificity. A recent meta-analysis of 16 clinical trials including 364 PD cases and 231 healthy controls found that NM-MRI had a sensitivity of 97.7% and a specificity of 94.4% to detect changes in the SN.

We believe our technique will improve the diagnostic accuracy of NM-MRI. NM-MRI without comparison to a control database; may have less diagnostic accuracy as a biomarker in very early stages of PD due to variability in NM levels in the SN in these patients as well as for normal controls. Accuracy of NM-MRI would be greatly enhanced by longitudinal assessments over time showing a decrease in NM in the SN over time. This outcome would not occur in non-PD patients.

Objectives

To demonstrate that NM levels in the SN will decrease over time in probable PD and early PD subjects using NM-MRI

To improve the accuracy of sensitivity/specificity of NM-MRI as a diagnostic biomarker as well as predicting progression of PD.

Rationale

The rationale is that NM-MRI can be used as a proxy measure for dopamine function in the SN and that lower values of NM in the SN are observed in PD patients and that those values will continue to decrease over time.

Study Design

The multi-center NM-MRI study will be conducted to evaluate over time SN and LC NM levels in approximately 200 early PD subjects with 100 control subjects. NM-MRI assessments will be made every 6 months for up to 2 years. Subjects will also be evaluated every 6 months which will include the Unified Parkinson's Disease Rating Scale (UPDRS) and concomitant medication.

Total sample size: Approximately 300 subjects

Duration of study: 2 years

Enrollment period: 1.5 years

Number of sites: Approximately 40 sites

Primary Endpoint:

Change in absolute NM levels between subjects with probable Parkinson's disease and control subjects at baseline and at endpoint (2 years total).

Rate of NM decrease between subjects with probable Parkinson's disease and control subjects at baseline and at endpoint (2 years total)

### Embodiments Relevant to NM-MRI and Parkinson's Disease

Not all patients have neuromelanin levels decreased to the same degree in PD. In fact, as shown in Cassidy et al. 2019, some patients with PD have neuromelanin higher than healthy controls and the inverse in true as well as some healthy patients have lower neuromelanin levels than patients with PD. The software discussed herein is a medical device that is able to aid in the diagnosis of Parkinson's disease without the patient having a known baseline and in the absence of symptoms by comparing the patient's neuromelanin levels to that of a large population database. If the patient's neuromelanin levels are lower than that of what has been determined to be the cutoff (more than about 30-50% less) then the diagnosis of PD is supported.

In a second embodiment, the patient would receive serial neuromelanin scans every 5 years. If the rate of neuromelanin decrease in the patient exceeds a certain percentage (%) of neuromelanin loss per year (more than about 10-15%) then a diagnosis of PD is supported.

In a third method, if the total amount of neuromelanin on serial scans has decreased to less than about 30% of the patient's baseline neuromelanin than the patient will be determined to have Parkinson's.

### Example 2A—Diagnosis and Longitudinal Assessment of Parkinson's Disease Neuromelanin-MRI Longitudinal Assessment of NM-MRI Will Assist with the Diagnosis of Early Parkinson's Disease

Primary Objectives

To determine the absolute change and percent change from baseline in NM-concentration that would be necessary to diagnose Parkinson's Disease (PD) using longitudinal assessments of Terran Neuromelanin-MRI Voxel Based Analysis

To demonstrate a decrease in NM-concentration from baseline to endpoint, using Terran Neuromelanin-MRI Voxel Based Analysis, in subjects with early Parkinson's disease (Stage 1 or 2) or LRRK2 haplotype compared with the control subjects

To determine the difference (absolute and percent change) in neuromelanin levels (SNc and SNc subregion concentration, volume of NM in SNc and SNc subregions) from a control group that would allow a diagnosis of PD

To demonstrate that total NM concentration in substantia nigra pars Compacta (SNc), NM concentration in the SNc subregions, volume of NM in the total SNc, and volume of subregions of the SNc are lower in subjects with PD compared to the normal range of the control group

Secondary Objectives:

To demonstrate a correlation between NM-MRI assessments (SNc and SNc subregion concentration, volume of NM in SNc and SNc subregions) and MDS-Unified Parkinson's Disease Rating Scale (MDS-UPDRS) scores

To demonstrate a correlation between NM-MRI assessments (SNc and SNc subregion concentration, volume of NM in SNc and SNc subregions) with DaTscan imaging over 5 years

To demonstrate a correlation between NM-MRI assessments (SNc and SNc subregion concentration, volume of NM in SNc and SNc subregions) with Hoehn and Yahr Staging Scale

To demonstrate that the application of the Terran Neuromelanin-MRI Voxel Based Analysis will find specific voxels (termed PD Voxels) unique to each patient that correlate with their specific symptoms on MDS-UPDRS

To demonstrate a correlation of Parkinson's Voxels with Parkinson's symptoms as measured by MDS-UPDRS

Endpoints

NM-MRI Imaging Using Terran Neuromelanin-MRI Voxel Based Analysis

Percent change from baseline to endpoint on Terran Neuromelanin-MRI Voxel Based Analysis in the subjects with early PD (Stage 1 or 2) or LRRK2 haplotype compared to the control subjects.

Total NM concentration in substantia nigra pars Compacta (SNc), NM concentration in the SNc, volume of NM in the total SNc, and volume of subregions of the SNc

Change from baseline to endpoint on Terran Neuromelanin-MRI Voxel Based Analysis in the subjects with early PD (Stage 1 or 2) or LRRK2 haplotype compared to the control subjects.

Total NM concentration in substantia nigra pars Compacta (SNc), NM concentration in the SNc, volume of NM in the total SNc, and volume of subregions of the SNc.

Difference (absolute and percent change) in neuromelanin levels (SNc and SNc subregion concentration, volume of NM in SNc and SNc subregions) from a control group (as it relates to diagnosis of PD).

Total NM concentration in substantia nigra pars Compacta (SNc), NM concentration in the SNc, volume of NM in the total SNc, and volume of subregions of the SNc in subjects with PD compared to the control group.

Correlation between NM-MRI assessments (SNc and SNc subregion concentration, volume of NM in SNc and SNc subregions) with Hoehn and Yahr Staging Scale.

PD Voxels in subjects with PD vs control subjects as it correlates to the MDS-UPDRS and specific symptoms on the MDS-UPDRS.

DaTscan imaging: 1) Diagnosis of PD; 2) Correlation with Terran Neuromelanin-MRI Voxel Based Analysis at baseline and over 5 years.

MDS-Unified Parkinson's Disease Rating Scale (MDS-UPDRS): 1) Correlation with Terran Neuromelanin-MRI Voxel Based Analysis at baseline and over 5 years; 2) Correlation with PD Voxels.

Study Design

The study is a 6-year study (1 year for recruitment and 5 years of follow-up) to demonstrate the diagnostic value of Terran Neuromelanin-MRI Voxel Based Analysis in subjects with early PD (Stage 1 or 2) who have not been treated with L-Dopa or are age >55 with LRRK2 haplotype who are asymptomatic. The study will enroll approximately 300 subjects with early PD (Stage 1 or 2) who have not been treated with L-Dopa or asymptomatic subjects with LRRK2 haplotype (age >55 years) and 200 age and gender matched controls. Subjects will be enrolled in the study after signing the informed consent form (ICF) and meeting all of the inclusion/exclusion criteria. During the screening period they will have a NM-MRI scan, DaTscan, and MDS-UPDRS. Each subject will be assessed using the Yahr Staging Scale. Subject will complete the same battery of tests after 12, 24, 36, 48, and 60 months after entry into the study.

Subject Population

At the completion of the screening procedures, patients must meet the following inclusion and not meet the exclusion criteria to be enrolled in the study.

Inclusion Criteria

Females and males over the age of 40 with early PD (Stage 1 or 2) who have not been treated with L-Dopa. Clinical diagnosis of PD will be confirmed with DaTscan.

Females and males age >55 with LRRK2 haplotype who are asymptomatic and not diagnosed with PD.

Consent to participate in the study and have the capacity to provide informed consent.

Exclusion Criteria

History of treatment with L-DOPA

History of DSM-V defined substance use disorder (except tobacco) for at least 6 months prior to screening or a positive urine drug screen (for amphetamines, cocaine, opioids, and phencyclidine). Subjects with mild, Cannabis or alcohol substance use disorder can be enrolled with the permission of the medical monitor

Claustrophobia or metal implants or paramagnetic objects contained within the body which may interfere with the MRI scan, as determined according to the guidelines set forth in the following reference book: “Guide to MR procedures and metallic objects” Shellock, PhD, Lippincott-Raven press, NY 1998.

Moderate or Severe Renal Disease

Allergy or hypersensitivity to iodine or DaTscanHistory of DSM-V defined substance use disorder (except tobacco) for at least 6 months prior to screening or a positive urine drug screen (for amphetamines, cocaine, opioids, and phencyclidine). Subjects with mild, Cannabis or alcohol substance use disorder can be enrolled with the permission of the medical monitor

Claustrophobia or metal implants or paramagnetic objects contained within the body which may interfere with the MRI scan, as determined according to the guidelines set forth in the following reference book: “Guide to MR procedures and metallic objects” Shellock, PhD, Lippincott-Raven press, NY 1998.

Moderate or Severe Renal Disease

Allergy or Hypersensitivity to Iodine or DatScan

Assessments—Screening:
ICF signed
Mini-International Neuropsychiatric Interview (MINI)
Psychiatric and Medical history
Demographics
Physical examination including height and body weight without shoes
Urine drug screen
Concomitant medications
Eligibility criteria
NM-MRI imaging
DaTscan
Hoehn and Yahr Staging Scale
MDS-UPDRS

Study Phase (Visits on Month 12, 24, 36, 48 and 60)

The following assessments are scheduled:
NM-MRI imaging
DaTscan
MDS-UPDRS

Statistical Analysis:

The study will enroll approximately 300 subjects with early PD (Stage 1) with no history of L-Dopa treatment or asymptomatic subjects with LRRK2 haplotype (age >55 years) and 200 age and gender matched controls. Sample size is based on a 20% decrease in SNc neuromelanin concentration levels at endpoint (after 5 years) in the subjects with Stage 1 PD or LRRK2 haplotype compared with the control subjects. The analysis for the primary and secondary endpoints will be done by Analysis of Covariance (ANCOVA), linear regression or Pearson correlation.

Primary Endpoints:

Percent change from baseline to endpoint on NM-MRI in the subjects with early PD (Stage 1 or 2) who have not been treated with L-Dopa or LRRK2 haplotype compared with the control subjects

Total NM concentration (microgram neuromelanin per microgram wet tissue) in substantia nigra pars Compacta (SNc), NM concentration in the SNc, volume of NM in the total SNc, and volume of subregions of the SNc

Change from baseline to endpoint on NM-MRI in the subjects with early PD (Stage 1 or 2) who have not been treated with L-Dopa or LRRK2 haplotype compared with the control subjects

Total NM concentration in substantia nigra pars Compacta (SNc), NM concentration in the SNc, volume of NM in the total SNc, and volume of subregions of the SNc

Total NM concentration in substantia nigra pars Compacta (SNc), NM concentration in the SNc, volume of NM in the total SNc, and volume of subregions of the SNc are lower in subjects with early PD compared to the normal range of the control group

To determine the difference (absolute and percent change) in neuromelanin levels (SNc concentration, volume of NM in SNc) in LRRK2 subjects from a control group that would allow a diagnosis of PD as confirmed by the DaTscan

Secondary Endpoints

Correlation between NM-MRI assessments and MDS-Unified Parkinson's Disease Rating Scale (MDS-UPDRS) scores and with DaTscan imaging over 5 years

Correlation between NM-MRI assessments (SNc and SNc subregion concentration, volume of NM in SNc and SNc subregions) with Hoehn and Yahr Staging Scale

Correlation of Parkinson's Voxels (identified by Terran Neuromelanin-MRI Voxel Based Analysis) with Parkinson's symptoms as measured by UPDRS

Identification of Parkinson's Voxels (identified by Terran Neuromelanin-MRI Voxel Based Analysis) that correlates with specific Parkinson's symptoms as measured by UPDRS

The exemplary voxel-based analysis procedure based on the dopamine biomarker neuromelanin can be used to detect Parkinson's disease in patients. There are currently no approved imaging tests that are able to diagnose Parkinson's disease, differentiate between different stages of Parkinson's disease, predict the course and/or progression of Parkinson's disease symptoms, predict future response to treatment, or predict future symptoms to in high risk individuals. The exemplary system, method, and computer-accessible medium can be performed with a standard hospital MRI machine. The exemplary voxel-based procedure, when method applied to NM-MRI, can be used as a biomarker in patients with Parkinson's disease in the clinical setting. The exemplary system, method, and computer-accessible medium can also be used to predict conversion of symptoms in people who are at high risk. Additionally, the exemplary system, method, and computer-accessible medium can be used to diagnose or predict the development of Parkinson's disease.

Neuromelanin-sensitive MRI (NM-MRI) can detect the content of neuromelanin (NM), a product of dopamine metabolism that accumulates with age in dopamine neurons of the substantia nigra (SN). Since NM-MRI can measure dopamine cell degeneration in the SN, this technique may be a useful marker of diagnosis of Parkinson's disease (PD) and may have utility for other neuropsychiatric conditions.

Parkinson's disease is a debilitating neurodegenerative illness that impairs motor control and cannot be adequately treated by current methods. Although the biological changes underlying the illness are known to involve degeneration of catecholaminergic neurons (dopamine and norepinephrine neurons), this degeneration cannot be precisely measured using current clinical tools. NM-MRI sequences purportedly are able to detect the neurochemical neuromelanin which is present in certain structures in the midbrain, namely the substantia nigra pars Compacta (SNc; containing dopamine neurons) and locus coeruleus (LC; containing norepinephrine neurons).

Recent evidence in PD suggests that dysfunction in the metabolism of dopamine and its byproduct neuromelanin may contribute to degeneration of dopamine cells, specifically of neuromelanin-positive cells, in the SN. Compromised neuromelanin-containing dopamine cells degenerate as a consequence and are removed by microglia along with their neuromelanin granules (the only known biological process that removes NM from the tissue). Therefore, an MRI scan sensitive to neuromelanin should be able to detect degeneration of catecholaminergic neurons in PD. The NM-MRI procedure is a brief (8-minute) and non-invasive structural MRI scan. Unlike most structural MRI scans, this type of scan is sensitive to a particular neurochemical, neuromelanin, due to the tendency of this neurochemical to bind metals.

Therefore, neuromelanin influences T1 and T2 relaxation times and can be observed without exposing subjects to any exogenous contrast agents. The NM-MRI scan shows the SNc and LC as hyperintense regions in the midbrain. Marked reduction in the intensity and area of this signal are observed in PD both for the SNc and LC clearly indicating that this signal is able to detect the neural degeneration occurring in PD. NM-MRI has already been shown to outperform existing PD biomarkers. These measures have also been shown to significantly correlate with illness severity in PD and to yield high sensitivity and specificity (80-95%) in the detection of early PD. A recent meta-analysis of 16 clinical trials including 364 PD cases and 231 healthy controls found that NM-MRI had a sensitivity of 97.7% and a specificity of 94.4%.

Neuromelanin imaging has been compared to the FDA approved DaTscan (SPECT imaging using ioflupane ¹²³I radiotracer) in patients with Parkinson's disease, with studies showing a statistically significant correlation with DaTscan. Specifically, the volume of the neuromelanin-positive substantia nigra pars Compacta (SNc) region as well as the asymmetry index of neuromelanin-positive SNc volume showed significant correlation with specific binding ratio (SBR) of the DaTscan.

Most cases of Parkinson disease likely result from a complex interaction of environmental and genetic factors. These cases are classified as sporadic and occur in people with no apparent history of the disorder in their family. The cause of these sporadic cases remains unclear. Approximately 15 percent of people with Parkinson disease have a family history of this disorder. One mutation associated with PD is LRRK2, which is inherited in an autosomal dominant pattern. Mutations in LRRK2 are the most common genetic cause of late-onset Parkinson's disease (PD) identified to date. The penetrance of LRRK2 mutations is clearly age dependent, increasing from 17% at age 50 to 85% at age 70 years.

Terran Neuromelanin-MRI Voxel Based Analysis has been validated as a proxy measure of dopamine function in a study that correlated NM-MRI scans to NM concentration in postmortem midbrain tissue. NM-MRI scans of SN-containing midbrain sections were performed on seven post-mortem individuals without histopathology compatible with PD or PD-related syndromes (including absence of Lewy bodies composed of abnormal protein aggregates). After scanning, each specimen was dissected along gridline markings and NM concentration measured using biochemical separation and spectrophotometry determination. The averaged NM-MRI contrast-to-noise ratio (CNR) across voxels within the grid section was also calculated. Across all midbrain specimens, grid sections with higher NM-MRI CNR had higher tissue concentration of NM (β₁=0.56, t₁₁₄=3.36, P=0.001, mixed-effects model). As expected, hyperintensities were most apparent in grid sections corresponding with the NM-rich SN. of NM-MRI CNR to NM concentration in non-periaqueductal gray (PAG) area regions (β₁=1.03, t₁₁₂=5.51, P=10⁻⁷). Under this model, a 10% increase in NM-MRI CNR corresponds with an estimated increase of 0.10 μg of NM per mg of tissue.

Terran Neuromelanin-MRI Voxel Based Analysis is being developed as a standalone software as a medical device (SaMD) that measures neuromelanin levels obtained with NM-MRI. Terran Neuromelanin-MRI Voxel Based Analysis can be used to provide an accurate measure of neuromelanin concentrations and volumes in the substantia nigra. Neuromelanin is a proxy measure of dopaminergic neuronal activity which can be used as an aide to physicians assessing subjects with medical conditions that impact dopamine levels of the midbrain. This study will longitudinally assess subjects with early PD (Stage 1 or 2) without a history of L-Dopa treatment or asymptomatic subjects with LRRK2 haplotype over a 5-year period.

To determine the absolute change and percent change from baseline in NM-concentration that would be necessary to diagnose Parkinson's Disease (PD) using longitudinal assessments of Terran Neuromelanin-MRI Voxel Based Analysis

To demonstrate a decrease in NM-concentration from baseline to endpoint, using Terran Neuromelanin-MRI Voxel Based Analysis, in subjects with early Parkinson's disease (Stage 1 or 2) or LRRK2 haplotype compared with the control subjects

To determine the difference (absolute and percent change) in neuromelanin levels (SNc and SNc subregion concentration, volume of NM in SNc and SNc subregions) from a control group that would allow a diagnosis of PD

To demonstrate that total NM concentration in substantia nigra pars Compacta (SNc), NM concentration in the SNc subregions, volume of NM in the total SNc, and volume of subregions of the SNc are lower in subjects with PD compared to the normal range of the control group

Secondary Objectives:

To demonstrate a correlation between NM-MRI assessments (SNc and SNc subregion concentration, volume of NM in SNc and SNc subregions) and MDS-Unified Parkinson's Disease Rating Scale (MDS-UPDRS) scores and with DaTscan imaging over 5 years

To demonstrate a correlation between NM-MRI assessments (SNc and SNc subregion concentration, volume of NM in SNc and SNc subregions) with Hoehn and Yahr Staging Scale

To demonstrate that the application of the Terran Neuromelanin-MRI Voxel Based Analysis will find specific voxels (termed PD Voxels) unique to each patient that correlate with their specific symptoms on MDS-UPDRS

To demonstrate a correlation of Parkinson's Voxels with Parkinson's symptoms as measured by MDS-UPDRS

Terran Neuromelanin-MRI Voxel Based Analysis is an effective method to diagnosis PD, differentiate PD subjects from controls and track the progression of PD over time

Terran Neuromelanin-MRI Voxel Based Analysis will identify Parkinson's Voxels that correspond to Parkinson's symptoms as measured by the MDS-UPDRS

Study Design

The study is a 6-year study (1 year for recruitment and 5 years of follow-up) to evaluate the diagnostic value of NM-MRI in subjects with early PD (Stage 1 or 2) without a history of L-Dopa treatment or are age ≥55 with LRRK2 haplotype who are asymptomatic. The study will enroll approximately 300 subjects with Stage 1 PD or asymptomatic subjects with LRRK2 haplotype (age ≥55 years) and 200 age and gender matched controls. Subjects will be enrolled in the study after signing the informed consent form (ICF) and meeting all of the inclusion/exclusion criteria. During the screening period they will have a NM-MRI scan, DaTscan SPECT imaging and MDS-UPDRS over 3 visits. Each subject will be assessed using the Yahr Staging Scale. Subjects will complete the same battery of tests after 12, 24, 36, 48 and 60 months after entry into the study. At each visit the MDS-UPDRS and Hoehn and Yahr Staging Scale will be done.

Subjects who have a history of current drug abuse will be excluded from the study. Finally, subjects with unstable medical conditions or contraindications for MRI studies will also be excluded.

Schedule of Assessments

Assessments will be performed as per the study flow chart in

Table 1.

TABLE 1
Schedule of Assessments

| | Visits | | | | | |
|---|---|---|---|---|---|---|
| | Screening | Study Visits (yearly) | | | | |
| Visit | 1-3 | 4 | 5 | 6 | 7 | 8 |
| Informed Consent Form | x | | | | | |
| MINI | x | | | | | |
| Medical History | x | | | | | |
| Hoehn and Yahr Staging Scale | x | x | x | x | x | x |
| MRI Metal Screening Questionnaire | x | x | x | x | x | x |
| Vital Signs (blood pressure and heart | x | x | x | x | x | x |
| rate) | | | | | | |
| Urine Drug Screen | x | x | x | x | x | x |
| NM-MRI Scan | x | x | x | x | x | x |
| DaTscan | x | x | x | x | x | x |
| MDS-UPDRS | x | x | x | x | x | x |
| Concomitant Medications | x | x | x | x | x | x |
| Adverse Events | x | x | x | x | x | x |

Screening—Visit 1

The Screening phase can last up to 30 days during which demographic information, medical history, and informed consent will be obtained. Subjects fulfilling the inclusion and exclusion criteria may be accepted for enrollment. The nature and purpose of the investigation must be explained to the subject prior to initiating screening activities.

After the signing of the Informed Consent Form (ICF), the following demographic data should be collected and recorded: date of birth, age of the patient at informed consent, gender, ethnicity, and race. Study site personnel are to obtain a complete medical history from the patient during Screening and update as needed on Visit 1. Medical history will be obtained as well as vital signs (blood pressure and pulse) will be measured. Urine will be collected for a drug screen. Current and recent past history of medication use will be obtained. The Investigator or designee will determine whether the subject fulfills all the eligibility criteria for the study.

The following assessments will be conducted:
ICF signed
MINI
Demographics
Medical history
Hoehn and Yahr Staging Scale
UPDRS
MRI Metal Screening Questionnaire
Vital signs (blood pressure and heart rate)
Urine drug screen
Concomitant medications
Adverse events
Eligibility criteria

Screening Visits 2 & 3

During Screening Visits 2 & 3 the following tests will be performed:
NM-MRI imaging
DaTscan imaging
Vital signs (blood pressure and heart rate)
Urine drug screen
Concomitant medications
Adverse events

Study Period (Visits 4-8, on Month 12, 24, 36, 48 and 60)
NM-MRI imaging
DaTscan
Hoehn and Yahr Staging Scale
MDS-UPDRS
MRI Metal Screening Questionnaire
Vital signs (blood pressure and heart rate)
Urine drug screen
Concomitant medications
Adverse events

Study Assessments—MRI Screening

Before inclusion in the MRI experiment, all subjects will be screened to ensure their eligibility for MRI scanning. The screening questionnaire includes questions regarding inclusion/exclusion criteria, including the presence of ferromagnetic implants. If the subject has any metallic implants (i.e. metal heart valve, aortic clips, etc.) that are unsuitable for the scanner, the subject will not be included in our study. Inclusion and exclusion in our study will be determined by the PI and co-PIs in this study.

Study Assessments—MRI Procedures

The subjects will undergo a structural 3-Tesla MRI scan and neuromelanin-sensitive (structural) scan. Neither scan involves the use of exogenous contrasts. Total scanning time will typically be around 30 minutes and will not exceed 1 hour. Subjects may be asked to come back for an additional scan if the initial data were not usable. During the MRI scan, the subject will be placed in a supine position on the camera table. Head will be positioned, and a plastic head-holder will be used to decrease head movement during the scan. Participants will be given a squeeze ball and will be instructed to squeeze if they feel unwell or if there is any problem during scanning, so the MRI staff can stop scanning. The participants will be given over-the-ear headphones to reduce the noise of the MRI. All subjects will undergo a structural MRI scan at the beginning of the session to allow for anatomical co-registration. All participants will undergo a metal screening questionnaire before each scanning session.

DaTscan Procedures

DatScan (Ioflupane I 123 injections) is a FDA approved radiopharmaceutical indicated for striatal dopamine transporter visualization using single photon emission computed tomography (SPECT) brain imaging to assist in the evaluation of adults with suspected Parkinsonian syndromes (PS). Parkinsonian syndromes are associated with dopamine transporter (DAT) loss in the striata. Ioflupane I 123 is a radiopharmaceutical indicated for striatal DAT visualization using SPECT brain imaging to assist in the evaluation of adult patients with suspected PS. Ioflupane 1123 binds to the DaT protein on dopaminergic nigrostriatal neurons, a bundle of nerve fibers in the brain. In a normal scan, Ioflupane 1123 is distributed in the striata and appear as mirrored “comma” or crescent shapes. A decrease in Ioflupane I 123 activity results in a circular “period” or oval shape(s) and reduced image intensity on one or both sides.

Procedures
Prior to the scan each subject will be asked whether they have an allergy to iodine, have a history of kidney or liver disease and has current or past use of cocaine.
The staff will administer thyroid-blocking agent (eg, potassium iodide oral solution equivalent to 100 mg iodide or potassium perchlorate 400 mg) at least 1 hr before dosing
The imaging center will measure patient dose by a suitable radioactivity calibration system immediately prior to administration.
The recommended dose is 111-185 MBq (3-5 mCi) injected through an intravenous (IV) line into your arm.
Begin SPECT imaging using a gamma camera between 3 and 6 hr post-injection
The DaTscan once started takes approximately 30-45 minutes.

During the SPECT scan the subject will lie on a table and an imaging technologist will position their head in a headrest. A strip of tape or a flexible restraint may be placed around the subject's head to help prevent head movement during the scan. A camera will be positioned above the subject's head and they must remain very still for about 30 minute while images are taken.

DaTscan is excreted by kidneys and severe renal impairment may increase radiation exposure to patient and alter images.

Safety of DaTscan

Contraindications

Hypersensitivity to active substance, excipients, or iodine

MDS-United Parkinson's Disease Rating Scale

The MDS-UPDRS has four parts:

Part I (non-motor experiences of daily living)

Part II (motor experiences of daily living)

Part III (motor examination)

Part IV (motor complications)

Part I has two components and 13 questions: IA concerns a number of behaviors that are assessed by the investigator with all pertinent information from patients and caregivers, and IB is completed by the patient with or without the aid of the caregiver, but independently of the investigator. These sections can, however, be reviewed by the rater to ensure that all questions are answered clearly, and the rater can help explain any perceived ambiguities. Part II has 13 questions is designed to be a self-administered questionnaire like Part IB, but can be reviewed by the investigator to ensure completeness and clarity. Part III is the motor examination and has 18 assessments. Part I addresses motor complications and has 6 questions.

Hoehn and Yahr Stage

The Hoehn and Yahr Scale is used to measure how Parkinson's symptoms progress and the level of disability. The original scale has stages 1 to 5. The study will use the modified scale which added Stage 0.

Stage 0—No signs of disease

Stage 1—Symptoms on one side only (unilateral)

Stage 2—Symptoms on both sides but no impairment of balance

Stage 3—Balance impairment, mild to moderate disease, physically independent

Stage 4—Severe disability, but still able to walk or stand unassisted

Stage 5—Needing a wheelchair or bedridden unless assisted

Statistical Analysis

Number of Subjects and Sample Size Calculation

The study will enroll approximately 300 subjects with Stage 1 PD or asymptomatic subjects with LRRK2 haplotype (age ≥55 years) and 200 age and gender matched controls. Sample size is based on a 20% decrease in substantia nigra neuromelanin levels at endpoint (after 5 years) in the subjects with Stage 1 PD or LRRK2 haplotype compared with the control subjects.

Secondary endpoints with be the correlation between NM-MRI scans and both DaTscan scan and UPDRS scores.

Analysis Populations

The following patient populations will be used for the statistical analyses:

Subjects who had one post screening NM-MRI scan

Statistical Methods

The study will enroll approximately 300 subjects with early PD (Stage 1 or 2) with no history of L-Dopa treatment or asymptomatic subjects with LRRK2 haplotype (age >55 years) and 200 age and gender matched controls. Sample size is based on a 20% decrease in substantia nigra neuromelanin levels at endpoint (after 5 years) in the subjects with early PD (Stage 1 or 2) or LRRK2 haplotype compared with the control subjects.

Subject Disposition

The number of subjects enrolled, completed, or discontinued from the study and the reason for study discontinuation will be tabulated by treatment group as appropriate.

Primary Endpoint

The study enrolls approximately 300 subjects with early PD (Stage 1 or 2) who have not been treated with L-Dopa or asymptomatic subjects with LRRK2 haplotype (age >55 years) and 200 age and gender matched controls. Sample size is based on a 20% decrease in SNc neuromelanin concentration levels at endpoint (after 5 years) in the subjects with Stage 1 PD or LRRK2 haplotype compared with the control subjects. The analysis for the primary and secondary endpoints is done by Analysis of Covariance (ANCOVA), linear regression or Pearson correlation.

Percent change from baseline to endpoint on NM-MRI in the subjects with early PD (Stage 1 or 2) who have not been treated with L-Dopa or LRRK2 haplotype compared with the control subjects

Total NM concentration (microgram neuromelanin per microgram wet tissue) in substantia nigra pars Compacta (SNc), NM concentration in the SNc, volume of NM in the total SNc, and volume of subregions of the SNc

Change from baseline to endpoint on NM-MRI in the subjects with early PD (Stage 1 or 2) who have not been treated with L-Dopa or LRRK2 haplotype compared with the control subjects

Total NM concentration in substantia nigra pars Compacta (SNc), NM concentration in the SNc, volume of NM in the total SNc, and volume of subregions of the SNc

Total NM concentration in substantia nigra pars Compacta (SNc), NM concentration in the SNc, volume of NM in the total SNc, and volume of subregions of the SNc are lower in subjects with early PD compared to the normal range of the control group

To determine the difference (absolute and percent change) in neuromelanin levels (SNc concentration, volume of NM in SNc) in LRRK2 subjects from a control group that would allow a diagnosis of PD as confirmed by the DaTscan

Secondary Endpoints

Correlation between NM-MRI assessments and MDS-Unified Parkinson's Disease Rating Scale (MDS-UPDRS) scores and with DaTscan imaging over 5 years

Correlation of Parkinson's Voxels (identified by Terran Neuromelanin-MRI Voxel Based Analysis) with Parkinson's symptoms as measured by MDS-UPDRS

Correlation between NM-MRI assessments (SNc and SNc subregion concentration, volume of NM in SNc and SNc subregions) with Hoehn and Yahr Staging Scale

Identification of Parkinson's Voxels (identified by Terran Neuromelanin-MRI Voxel Based Analysis) that correlates with specific Parkinson's symptoms as measured by MDS-UPDRS

To demonstrate the correlation between the change in neuromelanin measures after initiation of L-DOPA therapy and improvement in MDS-UPDRS scores

Level of neuromelanin increases (SNc concentration, volume of NM in SNc), as measured by Terran Neuromelanin-MRI Voxel Based Analysis, that results in improvement in MDS-UPDRS with L-Dopa therapy

### Example 2B—Assessment of Carbidopa/Levodopa Treatment in Parkinson's Disease with Terran Neuromelanin-MRI Voxel Based Analysis

Terran Neuromelanin-MRI Voxel Based Analysis is being developed as a standalone software as a medical device (SaMD) that measures neuromelanin levels obtained with NM-MRI. Terran Neuromelanin-MRI Voxel Based Analysis can be used to provide an accurate measure of neuromelanin concentrations and volumes in the substantia nigra. Neuromelanin is a proxy measure of dopaminergic neuronal activity which can be used as an aide to physicians assessing subjects with medical conditions that impact dopamine levels of the midbrain. This study will assess the effectiveness of carbidopa/levodopa treatment in the treatment of PD using Terran Neuromelanin-MRI Voxel Based Analysis.

Primary Objectives

To demonstrate the correlation between neuromelanin measures (substantia nigra pars Compacta (SNc), NM concentration in the SNc subregions, volume of NM in the total SNc, and volume of subregions of the SNc) and improvement in MDS-Unified Parkinson's Disease Rating Scale (MDS-UPDRS) scores after initiation of carbidopa/levodopa treatment

To determine the level of neuromelanin increases (SNc and SNc subregion concentration, volume of NM in SNc and SNc subregions), as measured by Terran Neuromelanin-MRI Voxel Based Analysis, that results in improvement in MDS-UPDRS to validate that NM levels can be used to monitor response to treatment

Terran Neuromelanin-MRI Voxel Based Analysis is an effective method to determine if a patient has an optimal response to carbidopa/levodopa treatment

Study Design

The study is a 12-week study in symptomatic PD subjects with no history of carbidopa/levodopa treatment. The study will demonstrate the value of Terran Neuromelanin-MRI Voxel Based Analysis to monitor subjects with PD who are initiating carbidopa/levodopa treatment. The study will enroll approximately 100 subjects with symptomatic PD who have not been treated with carbidopa/levodopa. Subjects will be enrolled in the study after signing the informed consent form (ICF) and meeting all of the inclusion/exclusion criteria. During the screening period they will have a NM-MRI scan using Terran Neuromelanin-MRI Voxel Based Analysis and MDS-UPDRS. Each subject will be assessed using the Yahr Staging Scale. Subject will have repeat NM-MRI scans and MDS-UPDRS assessments at 4 weeks, 8 weeks, and 12 weeks. After completing Screening, subjects will enter the Treatment Period and carbidopa/levodopa treatment will be initiated. The dose of carbidopa/levodopa will be individualized by the study clinician based on clinical response and adverse events. Dosage is best initiated with one tablet of carbidopa/levodopa 25 mg-100 mg three times a day. This dosage schedule provides 75 mg of carbidopa per day. Dosage may be increased by one tablet every day or every other day, as necessary, until a dosage of eight tablets of 25 mg-100 mg a day is reached. Extended release formulations of carbidopa/levodopa are permitted with similar dosing. Dose increase and decrease will be guided by NM-MRI signal comparisons.

Subjects who have a history of current drug abuse will be excluded from the study. Finally, subjects with unstable medical conditions or contraindications for MRI studies will also be excluded.

Schedule of Assessments

Assessments will be performed as per the study flow chart in Table 1.

TABLE 2
Schedule of Assessments

| | Visits | | | |
|---|---|---|---|---|
| | Screening | | Study Visits | |
| | | 3 | 4 | 5 |
| | 1 & | (Week | (Week | (Week |
| Visit | 2 | 4) | 8) | 12) |
| Informed Consent Form | x | | | |
| MINI | x | | | |
| Medical History | x | | | |
| Hoehn and Yahr Staging Scale | x | | | |
| MRI Metal Screening | x | x | x | x |
| Questionnaire | | | | |
| Vital Signs (blood pressure | x | x | x | x |
| and heart rate) | | | | |
| Urine Drug Screen | x | x | x | x |
| NM-MRI Scan | x | x | x | x |
| UPDRS | x | x | x | x |
| Concomitant Medications | x | x | x | x |
| Adverse Events | x | x | x | x |

Screening—Visit

The Screening phase can last up to 30 days during which demographic information, medical history, and informed consent will be obtained. Subjects fulfilling the inclusion and exclusion criteria may be accepted for enrollment. The nature and purpose of the investigation must be explained to the subject prior to initiating screening activities.

After the signing of the Informed Consent Form (ICF), the following demographic data should be collected and recorded: date of birth, age of the patient at informed consent, gender, ethnicity, and race. Study site personnel are to obtain a complete medical history from the patient during Screening and update as needed on Visit 1. Medical history will be obtained as well as vital signs (blood pressure and pulse) will be measured. Urine will be collected for a drug screen. Current and recent past history of medication use will be obtained. The Investigator or designee will determine whether the subject fulfills all the eligibility criteria for the study.

The following assessments will be conducted:
ICF signed
MINI
Demographics
Medical history
Hoehn and Yahr Staging Scale
UPDRS
MRI Metal Screening Questionnaire
Vital signs (blood pressure and heart rate)
Urine drug screen
Concomitant medications
Adverse events
Eligibility criteria

Screening Visits 2

During Screening Visits 2 the following tests will be performed:
NM-MRI imaging

Study Assessments

MRI Procedures

MRI Screening

Before inclusion in the MRI experiment, all subjects will be screened to ensure their eligibility for MRI scanning. The screening questionnaire includes questions regarding inclusion/exclusion criteria, including the presence of ferromagnetic implants. If the subject has any metallic implants (i.e. metal heart valve, aortic clips, etc.) that are unsuitable for the scanner, the subject will not be included in our study. Inclusion and exclusion in our study will be determined by the PI and co-PIs in this study.

MRI Procedures

The subjects will undergo a structural 3-Tesla MRI scan and neuromelanin-sensitive (structural) scan. Neither scan involves the use of exogenous contrasts. Total scanning time will typically be around 30 minutes and will not exceed 1 hour. Subjects may be asked to come back for an additional scan if the initial data were not usable. During the MRI scan, the subject will be placed in a supine position on the camera table. Head will be positioned, and a plastic head-holder will be used to decrease head movement during the scan. Participants will be given a squeeze ball and will be instructed to squeeze if they feel unwell or if there is any problem during scanning, so the MRI staff can stop scanning. The participants will be given over-the-ear headphones to reduce the noise of the MRI. All subjects will undergo a structural MRI scan at the beginning of the session to allow for anatomical co-registration. All participants will undergo a metal screening questionnaire before each scanning session.

MDS-United Parkinson's Disease Rating Scale

The MDS-UPDRS has four parts:
Part I (non-motor experiences of daily living)
Part II (motor experiences of daily living)
Part III (motor examination)
Part IV (motor complications)

Part I has two components and 13 questions: IA concerns a number of behaviors that are assessed by the investigator with all pertinent information from subjects and caregivers, and IB is completed by the patient with or without the aid of the caregiver, but independently of the investigator. These sections can, however, be reviewed by the rater to ensure that all questions are answered clearly, and the rater can help explain any perceived ambiguities. Part II has 13 questions is designed to be a self-administered questionnaire like Part IB, but can be reviewed by the investigator to ensure completeness and clarity. Part III is the motor examination and has 18 assessments. Part I addresses motor complications and has 6 questions.

Hoehn and Yahr Stage

The Hoehn and Yahr Scale is used to measure how Parkinson's symptoms progress and the level of disability. The original scale has stages 1 to 5. The study will use the modified scale which added Stage 0.
Stage 0—No signs of disease
Stage 1—Symptoms on one side only (unilateral)
Stage 2—Symptoms on both sides but no impairment of balance
Stage 3—Balance impairment, mild to moderate disease, physically independent
Stage 4—Severe disability, but still able to walk or stand unassisted
Stage 5—Needing a wheelchair or bedridden unless assisted.

Subject Enrollment and Withdrawal

Inclusion Criteria

To be eligible for participation in this study, a subject must meet all of the following inclusion criteria:

Females and males over the age of 40 with symptomatic PD without a history of carbidopa/levodopa or levodopa treatment.

Consent to participate in the study and have the capacity to provide informed consent

Exclusion Criteria

Subjects will be excluded from the study if they meet any of the following exclusion criteria:

History of treatment with carbidopa/levodopa or levodopa

History of DSM-V defined substance use disorder (except tobacco) for at least 6 months prior to screening or a positive urine drug screen (for amphetamines, cocaine, opioids, and phencyclidine). Subjects with mild, Cannabis or alcohol substance use disorder can be enrolled with the permission of the medical monitor

Claustrophobia or metal implants or paramagnetic objects contained within the body which may interfere with the MRI scan, as determined according to the guidelines set forth in the following reference book: “Guide to MR procedures and metallic objects” Shellock, PhD, Lippincott-Raven press, NY 1998.

Prior and Concomitant Therapy

All medications, including over-the-counter preparations and home remedies used by the patient within one month of entry into the study must be recorded on the case report form.

Safety

This is a non-interventional study to assess NM-MRI longitudinally. The assessment involves a standard 3-T MRI and software that measures the amount of neuromelanin in specific structures of the brain. Vital signs (blood pressure and pulse) will be measured at each study visit.

At the beginning of each visit study staff will ask a series of questions from a “Mini Check-Up” form that inquiries about hospitalization, medical condition, doctor visits, medication use, drug use and whether they were involved in an accident involving metal. Should information on adverse experiences be elicited during this questioning, the information must be recorded in the Case Report Form (CRF).

Note, given that this is a non-interventional study, physical examination, laboratory assessments, electrocardiogram tests will not be performed.

Vital Sign Measurements

Blood pressure (systolic and diastolic) will be measured at each visit, with the subject in the sitting position according to AHA recommendations. Pulse rate can be determined by palpation of radial pulse in the sitting position. Both blood pressure and pulse can be measured by a blood pressure monitor machine.

Risks to Subjects

Risks that could be encountered during the study period

All procedures will be virtually free of any risk or potential danger to participants. In most of the procedures there is a slight risk of discomfort due to boredom. Some questions in the assessment might refer to sensitive topics.

Magnetic Resonance Imaging

Both the FDA and the NYSPI IRB have deemed MRI Scanning to be classified as a non-significant risk. For this cross-scanner validation study the only risks that are applicable are those that have to do with the MRI scans (i.e., discomfort, fatigue, anxiety). MRI scanning will involve 3 Tesla scanners deemed to pose non-significant risk by the FDA.

In order to minimize risks and discomforts to participants, the site will:

Screen for metallic devices, implants, and other contraindications to scanning by using the MRI Metal Screening Questionnaire

Exclude pregnant subjects and conduct a urine pregnancy test prior to scanning.

Exclude subjects with claustrophobia. We will reduce this potential adverse reaction by discussing the procedure prior to entry into the magnet room, by providing the subject with a mirror through which they can look out into the room, and by communicating with the subject over the intercom. If a subject continues to feel uncomfortable, the imaging procedure is terminated and the subject is removed from the magnet.

Staff will provide adequate medical, safety monitoring, and observation during scanning

Study staff will be available to provide support, reduce anxiety, optimize the comfort of the subject, and remove the subject from the MRI machine, if requested

Carbidopa/Levodopa

All patients should be observed carefully for the development of depression with concomitant suicidal tendencies.

Carbidopa/levodopa should be administered cautiously to patients with severe cardiovascular or pulmonary disease, bronchial asthma, renal, hepatic or endocrine disease.

As with levodopa, care should be exercised in administering carbidopa/levodopa to patients with a history of myocardial infarction who have residual atrial, nodal, or ventricular arrhythmias.

As with levodopa, treatment with carbidopa/levodopa may increase the possibility of upper gastrointestinal hemorrhage in patients with a history of peptic ulcer.

Falling Asleep During Activities Of Daily Living And Somnolence: Patients taking carbidopa/levodopa alone or with other dopaminergic drugs have reported suddenly falling asleep without prior warning of sleepiness while engaged in activities of daily living (includes operation of motor vehicles). Patients should be advised to exercise caution while driving or operating machines during treatment with carbidopa/levodopa. Patients who have already experienced somnolence or an episode of sudden sleep onset should not participate in these activities during treatment with carbidopa/levodopa.

Before initiating treatment with carbidopa/levodopa, advise patients about the potential to develop drowsiness and ask specifically about factors that may increase the risk for somnolence with carbidopa/levodopa such as the use of concomitant sedating medications and the presence of sleep disorders.

Hyperpyrexia and confusion: Sporadic cases of a symptom complex resembling neuroleptic malignant syndrome.

Adverse Reactions/Events

The most common adverse reactions reported with carbidopa/levodopa have included dyskinesias, such as chloroform, dystonic, and other involuntary movements, and nausea.

The following other adverse reactions have been reported with carbidopa/levodopa:

Body As A Whole: Chest pain, asthenia.

Cardiovascular: Cardiac irregularities, hypotension, orthostatic effects including orthostatic hypotension, hypertension, syncope, phlebitis, palpitation.

Gastrointestinal: Dark saliva, gastrointestinal bleeding, development of duodenal ulcer, anorexia, vomiting, diarrhea, constipation, dyspepsia, dry mouth, taste alterations.

Hematologic: Agranulocytosis, hemolytic and non-hemolytic anemia, thrombocytopenia, leukopenia.

Hypersensitivity: Angioedema, urticaria, pruritus, Henoch-Schonlein purpura, bullous lesions (including pemphigus-like reactions).

Musculoskeletal: Back pain, shoulder pain, muscle cramps.

Nervous System/Psychiatric: Psychotic episodes including delusions, hallucinations, and paranoid ideation, bradykinetic episodes (“on-off” phenomenon), confusion, agitation, dizziness, somnolence, dream abnormalities including nightmares, insomnia, paresthesia, headache, depression with or without development of suicidal tendencies, dementia, pathological gambling, increased libido including hypersexuality, impulse control symptoms.

Respiratory: Dyspnea, upper respiratory infection.

Skin: Rash, increased sweating, alopecia, dark sweat.

Urogenital: Urinary tract infection, urinary frequency, dark urine.

Laboratory Tests: Decreased hemoglobin and hematocrit; abnormalities in alkaline phosphatase, SGOT (AST), SGPT (ALT), LDH, bilirubin, BUN, Coombs test; elevated serum glucose; white blood cells, bacteria, and blood in the urine.

Adverse Events

Adverse events (AEs) will be collected during the study. For detailed information on collection, definition, categorization, and reporting of AEs/SAEs during the study, refer to Section [00457].

AEs will be monitored throughout the study and the following information recorded:

Verbatim Complaint

Whether the event was a treatment-emergent adverse event

Whether the event was a serious adverse event

Date and time of onset

Severity of the event

Relationship of the event to study drug

Action taken regarding the study drug due to the event

Clinical outcome of the event (resolved or ongoing). If resolved, provide the date of resolution

Adverse Events

Definition of AEs, Period of Observation, and Recording of AEs

An AE is any unfavorable or unintended sign, symptom, or disease, whether or not considered related to the study. Adverse event recording will begin at the time the informed consent form is signed. Thereafter, AEs will be ascertained by asking the patient how he/she has been since the last visit. Assessment should continue as needed to follow up an AE to its resolution or acceptable stabilization, consistent with the medical judgment of the Investigator.

Every attempt should be made to describe the AE in terms of a diagnosis. Once a clear diagnosis has been made, individual signs and symptoms shall not be recorded unless they represent atypical or extreme manifestations of the diagnosis, in which case they should be reported as separate events. Events leading up to a diagnosis should be retained. If a clear diagnosis cannot be established, each sign and symptom must be recorded individually.

The Investigator is responsible for ensuring that all adverse clinical experiences, whether observed by the Investigator or reported by the patient, are reported on the CRF and in the patient's medical record. The Investigator must assign the following AE attributes:

Adverse Event Diagnosis or Syndrome, if Known (if not Known, Signs or Symptoms)

Dates of Onset and Resolution

Severity (and/or Toxicity Per Protocol)

Assessment of relatedness to investigational product, and

Action Taken

Categorizing Intensity

For both AEs and SAEs, the Investigator must assess the severity/intensity of the event. The severity/intensity of AEs will be graded based upon the patient's symptoms as follows:

Mild—transient or mild discomfort; no limitation in activity; no medical intervention/therapy required

Moderate—mild to moderate limitation in activity, some assistance may be needed; no or minimal medical intervention/therapy required

Severe—marked limitation in activity, some assistance usually required; medical intervention/therapy required, hospitalization is possible

Life-threatening—extreme limitation in activity, significant assistance required; significant medical intervention/therapy required, hospitalization or hospice care probable

The term “severe” is often used to describe the intensity of a specific event (as in mild, moderate or severe myocardial infarction); the event itself, however, may be of relatively minor medical significance (such as severe headache). This criterion is not the same as “serious” which is based on patient/event outcome or action criteria associated with events that pose a threat to a patient's life or functioning.

Seriousness, not severity, serves as a guide for defining regulatory obligations.

Statistical Analysis

Number of Subjects and Sample Size Calculation

The study will enroll approximately 100 subjects with symptomatic PD with no history of carbidopa/levodopa treatment. Sample size is based on a 20% improvement in SNc neuromelanin concentration levels at endpoint after carbidopa/levodopa treatment.

Analysis Populations

The following patient populations will be used for the statistical analyses:

Subjects who had one post screening NM-MRI scan

Statistical Methods

Subject Disposition

The number of subjects enrolled, completed, or discontinued from the study and the reason for study discontinuation will be tabulated by treatment group as appropriate.

Primary Endpoints

Correlation of neuromelanin measures (substantia nigra pars Compacta (SNc), NM concentration in the SNc subregions, volume of NM in the total SNc, and volume of subregions of the SNc) with improvement in MDS-UPDRS scores after initiation of carbidopa/levodopa therapy

Neuromelanin levels (SNc and SNc subregion concentration, volume of NM in SNc and SNc subregions) that results in improvement in MDS-UPDRS after carbidopa/levodopa initiation

Details of the statistical analysis will be included in the Statistical Analysis Plan (SAP)

NM-MRI Analysis

Details of the analyses will be presented in the Statistical Analysis Plan.

Protocol Deviations

All deviations will be listed. Protocol deviations will be categorized as major or minor. Major protocol deviations will be determined by the Sponsor. Subjects with major protocol deviations, or data points that are judged to be major protocol deviations will be excluded from the PP Population.

Demographic and Baseline Characteristics

Demographics and baseline characteristics will be listed and summarized by treatment and overall.

Safety

Adverse Events

Type and incidence of adverse events will be tabulated.

AEs will be coded using the most current version of Medical Dictionary for Regulatory Activities (MedDRA®).

The number and percentage of subjects experiencing TEAEs, treatment-emergent SAEs, and TEAEs leading to study discontinuation will be summarized by treatment group and overall by MedDRA system organ class (SOC) and/or preferred term (PT).

Vital Signs

Descriptive statistics will be calculated for all vital sign measurements (blood pressure and pulse) change from baseline.

List of Abbreviations Used:

| Abbreviation | Definition |
|---|---|
| AE | Adverse Event |
| ANCOVA | Analysis of Covariance |
| CRF | Case Report Form |
| CNR | Contrast Noise Ratio |
| CoV | Coefficient of Variation |
| DAT | Dopamine Transporter |
| DSM-V | Diagnostic and Statistical Manual of Mental |
| | Disorders, Fifth Edition |
| FDA | Food and Drug Administration |
| GCP | Good Clinical Practice |
| ID | Identification |
| ICF | Informed Consent Form |
| ICH | International Conference on Harmonisation |
| LRRK2 | Leucine-Rich Repeat Kinase 2 |
| LC | Locus Coeruleus |
| MedDRA | Medical Dictionary for Regulatory Activities |
| MRI | Magnetic Resonance Imaging |
| MDS-UPDRS | Movement Disorder Society-Unified Parkinson's |
| | Disease Rating Scale |
| MINI | Mini International Neuropsychiatric Interview |
| NM | Neuromelanin |
| NM-MRI | Neuromelanin Magnetic Resonance Imaging |
| PD | Parkinson's Disease |
| PS | Parkinson's Syndromes |
| PAG | Periaqueductal Gray |
| PI | Principal Investigator |
| PT | Preferred Term |
| SAE | Serious Adverse Event |
| SPECT | Single Photon Emission Computed Tomography |
| SBR | Specific Binding Ratio |
| SN | Substantia Nigra |
| SNc | Substantia Nigra Compacta |
| SAP | Statistical Analysis Plan |
| SOC | System Organ Class |
| 3-T | 3-Tesla |
| TBD | To Be Determined |
| TEAE | Treatment Emergent Adverse Event |

### REFERENCES FOR EXAMPLE 2

The following references are hereby incorporated by reference in their entireties.
Sasaki M, Shibata E, Kudo K, Tohyama K. Neuromelanin-Sensitive MRI. Clinical Neuroradiology. 2008; 18(3):147-153.
Sasaki M, Shibata E, Tohyama K, et al. Neuromelanin magnetic resonance imaging of locus ceruleus and substantia nigra in Parkinson's disease. Neuroreport. 2006; 17(11):1215-1218.
Chen X, Huddleston D E, Langley J, et al. Simultaneous imaging of locus coeruleus and substantia nigra with a quantitative neuromelanin MRI approach. Magnetic resonance imaging. 2014; 32(10):1301-1306.
Kashihara K, Shinya T, Higaki F. Reduction of Neuromelanin-Positive Nigral Volume in Patients with MSA, PSP and CBD. Internal Medicine. 2011; 50(16):1683-1687.
Kashihara K, Shinya T, Higaki F. Neuromelanin magnetic resonance imaging of nigral volume loss in patients with Parkinson's disease. Journal of Clinical Neuroscience. 2011; 18(8):1093-1096.
Kitao S, Matsusue E, Fujii S, et al. Correlation between pathology and neuromelanin M R imaging in Parkinson's disease and dementia with Lewy bodies. Neuroradiology. 2013; 55(8):947-953.
Matsuura K, Maeda M, Yata K, et al. Neuromelanin magnetic resonance imaging in Parkinson's disease and multiple system atrophy. European neurology. 2013; 70 (1-2):70-77.
Miyoshi F, Ogawa T, Kitao S-i, et al. Evaluation of Parkinson Disease and Alzheimer Disease with the use of neuromelanin M R imaging and 123I-metaiodobenzylguanidine scintigraphy. American Journal of Neuroradiology. 2013; 34(11):2113-2118.
Ogisu K, Kudo K, Sasaki M, et al. 3D neuromelanin-sensitive magnetic resonance imaging with semi-automated volume measurement of the substantia nigra pars Compacta for diagnosis of Parkinson's disease. Neuroradiology. 2013; 55(6):719-724.
Ohtsuka C, Sasaki M, Konno K, et al. Differentiation of early-stage parkinsonisms using neuromelanin-sensitive magnetic resonance imaging. Parkinsonism & Related Disorders. 2014; 20(7):755-760.
Ohtsuka C, Sasaki M, Konno K, et al. Changes in substantia nigra and locus coeruleus in patients with early-stage Parkinson's disease using neuromelanin-sensitive M R imaging. Neurosci Lett. 2013; 541:93-98.
Schwarz S T, Rittman T, Gontu V, Morgan P S, Bajaj N, Auer D P. T1-weighted MRI shows stage-dependent substantia nigra signal loss in Parkinson's disease. Mov Disord. 2011; 26(9):1633-1638.
Garcia-Lorenzo D, Longo-Dos Santos C, Ewenczyk C, et al. The coeruleus/subcoeruleus complex in rapid eye movement sleep behaviour disorders in Parkinson's disease. Brain. 2013; 136 (Pt 7):2120-2129.
Tanaka M, Aihara Y, Ikeda S, Aihara Y. [Neuromelanin-related contrast in the substantia nigra semiquantitatively evaluated by magnetic resonance imaging at 3T: comparison between normal aging and Parkinson disease]. Rinsho Shinkeigaku. 2011; 51(1):14-20.
Castellanos G, Fernandez-Seara M A, Lorenzo-Betancor O, et al. Automated neuromelanin imaging as a diagnostic biomarker for Parkinson's disease. Mov Disord. 2015; 30(7):945-952.
Sasaki M, Shibata E, Ohtsuka K, et al. Visual discrimination among patients with depression and schizophrenia and healthy individuals using semiquantitative color-coded fast spin-echo T1-weighted magnetic resonance imaging. Neuroradiology. 2010; 52(2):83-89.
Shibata E, Sasaki M, Tohyama K, et al. Use of neuromelanin-sensitive MRI to distinguish schizophrenic and depressive patients and healthy individuals based on signal alterations in the substantia nigra and locus ceruleus. Biological psychiatry. 2008; 64(5):401-406.
Watanabe Y, Tanaka H, Tsukabe A, et al. Neuromelanin magnetic resonance imaging reveals increased dopaminergic neuron activity in the substantia nigra of patients with schizophrenia. PLoS One. 2014; 9 (8):e104619.
Sulzer D, Cassidy C, Horga G, et al. Neuromelanin detection by magnetic resonance imaging (MRI) and its promise as a biomarker for Parkinson's disease. NPJ Parkinson's disease. 2018; 4(1):11.
Zucca F A, Basso E, Cupaioli F A, et al. Neuromelanin of the human substantia nigra: an update. Neurotoxicity research. 2014; 25(1):13-23.
Zecca L, Fariello R, Riederer P, Sulzer D, Gatti A, Tampellini D. The absolute concentration of nigral neuromelanin, assayed by anew sensitive method, increases throughout the life and is dramatically decreased in Parkinson's disease. FEBS letters. 2002; 510(3):216-220.
Segura-Aguilar J, Paris I, Munoz P, Ferrari E, Zecca L, Zucca F A. Protective and toxic roles of dopamine in Parkinson's disease. J Neurochem. 2014; 129(6):898-915.
Viceconte N, Burguillos M A, Herrera A J, De Pablos R M, Joseph B, Venero J L. Neuromelanin activates proinflammatory microglia through a caspase-8-dependent mechanism. J Neuroinflammation. 2015; 12:5.
Enochs W S, Petherick P, Bogdanova A, Mohr U, Weissleder R. Paramagnetic metal scavenging by melanin: M R imaging. Radiology. 1997; 204(2):417-423.
Tosk J M, Holshouser B A, Aloia R C, et al. Effects of the interaction between ferric iron and L-dopa melanin on T1 and T2 relaxation times determined by magnetic resonance imaging. Magn Reson Med. 1992; 26(1):40-45.
Blazejewska Al, Schwarz S T, Pitiot A, et al. Visualization of nigrosome 1 and its loss in P D: pathoanatomical correlation and in vivo 7 T MRI. Neurology. 2013; 81(6):534-540.
Castellanos G, Fernández-Seara M A, Lorenzo-Betancor O, et al. Automated Neuromelanin Imaging as a Diagnostic Biomarker for Parkinson's Disease. Movement Disorders. 2015; 30(7):945-952.
Kuya K, Shinohara Y, Miyoshi F, Fujii S, Tanabe Y, Ogawa T. Correlation between neuromelanin-sensitive M R imaging and 123 I-FP-CIT SPECT in patients with parkinsonism. Neuroradiology. 2016; 58(4):351-356.
Isaias I U, Trujillo P, Summers P, et al. Neuromelanin imaging and dopaminergic loss in Parkinson's disease. Frontiers in aging neuroscience. 2016; 8:196.
Mahlknecht P, Krismer F, Poewe W, Seppi K. Meta-analysis of dorsolateral nigral hyperintensity on magnetic resonance imaging as a marker for Parkinson's disease. Movement Disorders. 2017; 32(4):619-623.
Bae Y J, Kim J M, Kim E, et al. Loss of nigral hyperintensity on 3 Tesla MRI of parkinsonism: comparison with 123I-FP-CIT SPECT. Movement Disorders. 2016; 31(5):684-692.
Oh S W, Shin N Y, Lee J J, et al. Correlation of 3D FLAIR and dopamine transporter imaging in patients with parkinsonism. American Journal of Roentgenology. 2016; 207(5):1089-1094.
Paisán-Ruiz C, Lewis P A, Singleton A B. LRRK2: cause, risk, and mechanism. J Parkinsons Dis. 2013; 3(2):85-103.
Kachergus J, Mata I F, Hulihan M, et al. Identification of a novel LRRK2 mutation linked to autosomal dominant parkinsonism: evidence of a common founder across European populations. Am J Hum Genet. 2005; 76(4):672-680.
Cassidy C M, Zucca F A, Girgis R R, et al. Neuromelanin-sensitive MRI as a noninvasive proxy measure of dopamine function in the human brain. Proceedings of the National Academy of Sciences. 2019; 116(11):5108-5117.

### Example 3—Evidence for Dopamine Abnormalities in the Substantia Nigra in Cocaine Addiction Revealed by Neuromelanin-Sensitive MRI

Abstract

Objective: Recent evidence supports the use of neuromelanin-sensitive MRI (NM-MRI) as a novel tool to investigate dopamine function in the human brain. The goal of this study was to investigate the NM-MRI signal in cocaine use disorder, compared to age and sex-matched controls, based on previous imaging studies showing that this disorder is associated with blunted pre-synaptic striatal dopamine.

Methods: NM-MRI and T1-weighted images were acquired from 20 participants with cocaine use disorder and 35 controls. Diagnostic group effects in NM-MRI signal were determined using a voxelwise analysis within the substantia nigra (SN). A subset of 20 cocaine users and 17 controls also underwent functional MRI imaging using the Monetary Incentive Delay task, in order to investigate whether NM-MRI was associated with alterations in reward processing.

Results: Compared to controls, cocaine users showed significantly increased NM-MRI signal in ventrolateral regions of the SN (linear regression; corrected p=0.025, permutation test; area under the receiver-operating-characteristic curve=0.83). Exploratory analyses did not find a significant correlation of NM-MRI signal to activation of the ventral striatum during anticipation of monetary reward.

Conclusions: Given that previous imaging studies show decreased dopamine signaling in the striatum, the finding of increased NM-MRI signal in the SN provides additional insight into the pathophysiology of cocaine use disorder. One interpretation is that cocaine use disorder is associated with a redistribution of dopamine between cytosolic and vesicular pools, leading to increased accumulation of neuromelanin. The study thus suggests that NM-MRI can serve as a practical imaging tool for interrogating the dopamine system in addiction.

### Introduction

Alterations of dopamine function have been previously demonstrated in cocaine use disorder using Positron Emission Tomography (PET), including measures of dopamine uptake, receptor density, and dopamine release (1). The reduction of stimulant-induced pre-synaptic dopamine release in cocaine users, measured with PET, is well replicated (1-4) and associated with more refractory symptoms of cocaine use disorder, including relapse (1, 2). However, while PET can provide important insights regarding dopamine signaling in addiction, it is costly and requires significant specialized infrastructure. Further, its use in longitudinal studies and research in younger, at-risk, populations is limited by radioactivity exposure.

Recent work suggests that neuromelanin-sensitive magnetic resonance imaging (NM-MRI) may provide a complementary noninvasive proxy measure of dopamine function and integrity (5, 6). Neuromelanin (NM) is a pigment generated from the conversion of cytosolic dopamine that accumulates gradually over the lifespan in dopamine neurons of the substantia nigra (SN) (7). Neuromelanin is bound to iron, forming paramagnetic complexes that can be imaged using MRI (6, 8, 9). NM-MRI can reliably capture neuromelanin depletion following SN neurodegeneration in Parkinson's disease (6, 10). Critically, this technique can also capture alterations in dopamine function in the absence of neurodegeneration (5, 11), consistent with in vitro evidence that stimulating dopamine synthesis boosts NM synthesis (12, 13).

In particular, NM-MRI signal within a subregion of the substantia nigra is increased in relation to psychosis (5), consistent with PET findings of increased dopamine signaling in psychosis (14). Furthermore, NM-MRI signal correlates directly with both PET measures of pre-synaptic dopamine release and resting blood flow in the midbrain (5). Thus, in one embodiment, the subject matter disclosed herein demonstrates that NM-MRI provides a proxy measure for functional changes in dopaminergic pathways with utility for studying psychiatric disorders without overt neurodegeneration.

Here, NM-MRI was employed for the first time to examine if similar changes could be detected in cocaine use disorder, a disorder involving dopamine dysfunction. To this end, the main analyses herein tested for effects of diagnostic group on NM-MRI signal in the substantia nigra. Without being bound by theory, based on previous PET studies (1, 3), it is thought that cocaine use disorder would be associated with reduced NM-MRI signal. In exploratory analyses, evaluated associations between changes in NM-MRI signal intensity in cocaine use disorder and hemodynamic brain responses during the Monetary Incentive Delay task were evaluated. Activation of the ventral striatum during the anticipation of reward in this task has been shown to provide a robust functional readout of reward processing (15) related to dopamine (16, 17) that is consistently reduced in drug and behavioral addictions (18, 19). Since the ventral striatum receives projections from ventral tegmental area and the dorsomedial SN (20, 21), the relationship between NM-MRI signal in the SN and reward related activation in ventral striatum was explored.

Methods

Participants

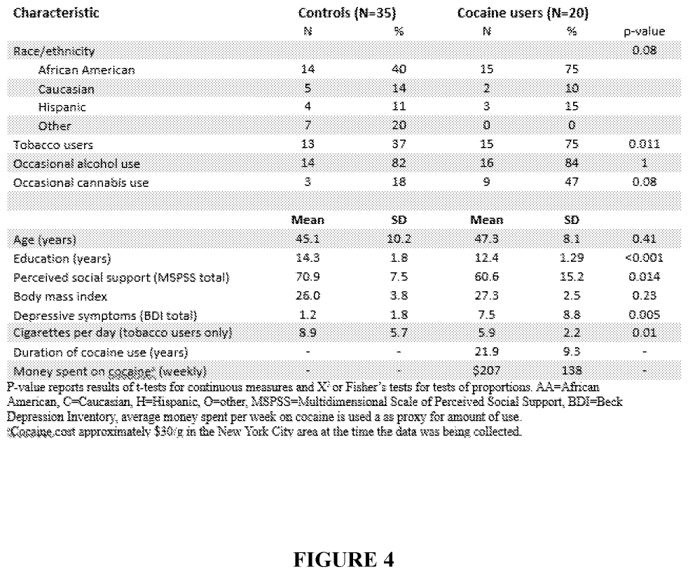

This study was approved by the Institutional Review Board of the New York State Psychiatric Institute. All participants provided written informed consent. The cocaine using participants met DSM-V criteria for moderate to severe cocaine use disorder with no other current Axis I diagnosis or current medical illness. Any other substance use disorder (aside from tobacco and cocaine) was an exclusion criterion. At the time of inclusion, these participants were actively using smoked cocaine, which was verified by urine toxicology. They were required to be abstinent for a minimum of 5 days prior to the scan, which was verified by urine drug testing (performed every other day). Participants refrained from tobacco use for one hour at minimum prior to scanning. A group of tobacco using and non-tobacco using controls was also included. Screening procedures included a physical exam, electrocardiogram, and laboratory tests. All participants were recruited through advertisements and by word-of-mouth. Controls were excluded for: current or past Axis I disorder (except tobacco use disorder), history of neurological disorders, or current major medical illness. In total, 58 males participated in the study. Three participants (1 cocaine user and 2 controls) were excluded due to unusable NM-MRI images (either due to participant motion [showing clearly visible, smearing or banding artifacts affecting the midbrain, n=2] or due to incorrect image-stack placement [n=1]). Thus, a total of 55 participants were retained for analysis: 20 cocaine users and 35 age and sex-matched controls as shown in FIG. 4. All participants completed self-report questionnaires including the Multidimensional Scale of Perceived Social Support (22) and the Beck Depression Inventory (23).

NM-MRI Acquisition

Magnetic resonance (MR) images were acquired for all study participants on a GE Healthcare 3T MR750 scanner using a 32-channel, phased-array Nova head coil following methods in prior work (5). For logistical reasons, a few scans (7% of all scans, 4 out of a total of 55) were acquired using an 8-channel In vivo head coil instead. NM-MRI images were acquired using a 2D gradient response echo sequence with magnetization transfer contrast (2D GRE-MT) with the following parameters: repetition time (TR)=260 ms; echo time (TE)=2.68 ms; flip angle=40°; in-plane resolution=0.39×0.39 mm2; partial brain coverage with field of view (FoV)=162×200; matrix=416×512; number of slices=10; slice thickness=3 mm; slice gap=0 mm; magnetization transfer frequency offset=1,200 Hz; number of excitations (NEX)=8; acquisition time=8.04 minutes. The slice-prescription protocol consisted of orienting the image stack along the anterior-commissure-posterior-commissure line and placing the top slice 3 mm below the floor of the third ventricle (for more detail, see (5)). This protocol provided coverage of SN-containing portions of the midbrain and surrounding structures. To support the preprocessing of NM-MRI images (see below), whole-brain, high-resolution T1-weighted structural MRI scans were also acquired using a fast spoiled gradient echo sequence (inversion time=500 ms, TR=6.37 ms, TE=2.59 ms, flip angle=11°, FoV=256×256, number of slices=244, isotropic voxel size=1.0 mm3) or, in some cases, a 3D BRAVO sequence (inversion time=450 ms, TR≈7.85 ms, TE≈3.10 ms, flip angle=12°, FoV=240×240, number of slices=220, isotropic voxel size=0.8 mm3). Quality of NM-MRI images was visually inspected for artifacts immediately after acquisition, and scans were repeated when necessary, time permitting.

NM-MRI Preprocessing

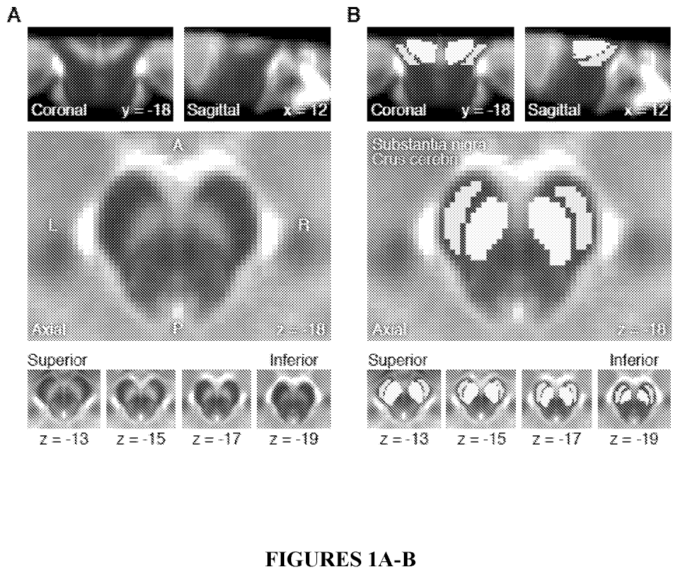

As in prior work (5), NM-MRI scans were preprocessed using SPM12 to allow for voxelwise analyses in standardized MI space. NM-MRI scans were first coregistered to participants' T1-weighted scans. Tissue segmentation was then performed using the T1-weighted images. NM-MRI scans were normalized to MNI space using DARTEL routines with a gray- and white-matter template generated from all study participants. The resampled voxel size of unsmoothed, normalized NM-MRI scans was 1 mm, isotropic. All images were visually inspected after each preprocessing step. Intensity normalization and spatial smoothing were then performed using custom Matlab (Mathworks) scripts. Contrast-to-noise ratio (CNR) for each participant and voxel v was calculated as the relative difference in NM-MRI signal intensity I from a reference region RR of white matter tracts known to have minimal NM content, the crus cerebri, as: CNRᵥ=(Iᵥ−mode(I_{RR}))/mode(I_{RR}). A template mask of the reference region and of the SN was created by manual tracing on a template NM-MRI image in MNI space (an average of normalized NM-MRI scans from all study participants, see FIG. 1 and previous report for more details (5)). The mode(I_{RR}) was calculated for each participant from a kernel-smoothing-function fit to a histogram of the distribution of all voxels in the mask. The resulting NM-MRI contrast-to-noise ratio maps were then spatially smoothed with a 1-mm full-width-at-half maximum Gaussian kernel.

NM-MRI Analysis

All analyses were carried out in Matlab. Following prior studies (5), the main analysis consisted of a voxelwise analysis of contrast-to-noise ratio values in the SN mask. This approach captures topographic alterations presumably corresponding with functionally distinct SN neuron subpopulations (20) and which previously showed high sensitivity to dopaminergic pathophysiology (5). In particular, the primary voxelwise analysis examined specific differences between cocaine users and controls via a robust linear regression analysis (robustfit function in Matlab) that predicted contrast-to-noise ratio (NM signal) at every voxel v within the SN mask as: CNR_{V}=β₀+β₁·diagnosis+Σᵢ₌₂ⁿβᵢ·nuisance covariate+ε, with tobacco use (cigarettes per day), head coil and age as nuisance covariates. Note that correcting for age is critical given the known relationship between age and neuromelanin accumulation (7). As in prior work (5), a group-derived template SN mask was used after censoring participant data points with missing values due to incomplete SN coverage or extreme values (contrast-to-noise ratio<−8% or contrast-to-noise ratio>40%; on average 71±195 voxels or 4% of all SN voxels were censored per subject). To correct for multiple comparisons and again following the prior work (5), the spatial extent of an effect was defined as the number of voxels k (adjacent or nonadjacent) exhibiting diagnostic differences (between cocaine users and controls) in NM signal in either the positive or the negative direction (voxel-level height threshold for t-test of regression coefficient β₁ of p<0.05, one-sided; note that the results remained significant at a more stringent height threshold of p<0.01). Significance testing was then determined based on a permutation test in which diagnosis labels were randomly shuffled with respect to individual maps of NM signal. This provided a measure of spatial extent for each of 10,000 permuted datasets, forming a null distribution against which to calculate the probability of observing the spatial extent k of the effect in the true data by chance. Thus, this test corrects for multiple comparisons by determining whether an effect's spatial extent k was greater than would be expected by chance (p_{corrected}<0.05; 10,000 permutations).

For a more detailed topographical description of the voxelwise effects in the SN, a post-hoc, multiple-linear regression analysis across SN voxels was used to predict the strength of an effect as a function of MNI voxel coordinates in the x (absolute distance from the midline), y, and z directions within the SN mask. For completeness, a region-of-interest analysis was also carried out on the average NM signal across the whole SN mask. This region-of-interest analysis consisted of a robust linear regression analysis including head coil, age, and incomplete SN coverage (yes/no) as nuisance covariates.

The ability of NM-MRI to segregate participants based on diagnostic group was determined by calculating effect size estimates and area under the receiver-operating-characteristic curve based on the mean NM-MRI signal in voxels identified in the primary voxelwise analysis to be relevant to cocaine use disorder (henceforth referred to as “cocaine-use voxels”: voxels showing a diagnosis effect via the primary voxelwise analysis or via a voxelwise analysis following a leave-one-out procedure. The leave-one-out procedure was employed to obtain an measure of effect size unbiased by voxel selection: for a given participant, voxels where the variable of interest was related to NM-MRI signal were first identified in an analysis including all participants except for this (held-out) participant. The mean signal in the held-out participant was then calculated from this set of voxels. This procedure was repeated for all participants so that each participant had an extracted, mean NM-MRI signal value obtained from an analysis that excluded them. Confidence intervals for Cohen's d and f² effect-size measures were determined by bootstrapping.

Partial correlations related clinical measures to NM-MRI signal extracted from cocaine-use voxels, with age and tobacco use as covariates. Partial (nonparametric) Spearman correlation was used because the clinical measures were not normally distributed according to a Lilliefors test at p<0.05.

fMRI methods

fMRI data were collected in 37 of the study participants (20 cocaine users, 17 controls). Blood oxygen level dependent (BOLD) fMRI was acquired while participants completed the Monetary Incentive Delay task. Echo planar images were acquired with the following parameters: repetition time (TR)=1500 ms; echo time (TE)=27 ms; flip angle=60°; in-plane resolution=3.5×3.5 mm²; slice thickness=4 mm; slice gap=1 mm. There were 2 runs each lasting 12.1 minutes. fMRI images were preprocessed using standard methods in SPM12 including slice-time correction, realignment, coregistration to the T1-weighted scans, spatial normalization to standardized MNI space, and smoothing (6 mm full-width at half maximum kernel). The Monetary Incentive Delay task employed was similar to a standard version (24) involving presentation of visual cues (geometric shapes) linked to subsequent receipt of feedback regarding monetary reward ($1 or $5), monetary loss ($1 or $5), or no outcome ($0). The task consisted of 110 trials equally divided into the 5 conditions. Earning money or avoiding losses was probabilistically achieved by having participants make fast key presses following the visual cue. The time available to make a key press was personalized based on participants' motor speed during practice testing. A first-level model included boxcar regressors for all 5 conditions during the anticipation period (defined as the period following button pressing and prior to feedback), the prospect period (following cue presentation and prior to button pressing), and the outcome period (when feedback was delivered). Nuisance regressors included 24 motion parameters (6 motion parameters and their squares, derivatives, and squared derivatives) and session-specific intercepts corresponding to the 2 runs. As in prior work (15), activation during reward anticipation was defined by the contrast between the $5 versus $0 gain conditions. For each participant, the signal from this contrast within a mask of the ventral striatum (from a publicly available functional mask of the striatum//osf.io/jkzwp/) was extracted. The ventral striatum is the brain structure most commonly investigated when using this task (19) and has been shown to provide a robust and reliable readout of reward-related activity during this task (25). To determine relationship to NM-MRI, a linear regression was used to investigate the effect of diagnosis, NM-MRI signal in cocaine-use voxels, and the interaction of diagnosis by NM-MRI signal on anticipatory BOLD activity in the ventral striatum controlling for age and tobacco use.

Results

Effect of Diagnosis on NM-MRI Signal in the Substantia Nigra

A priori voxelwise analysis of differences between cocaine users and controls

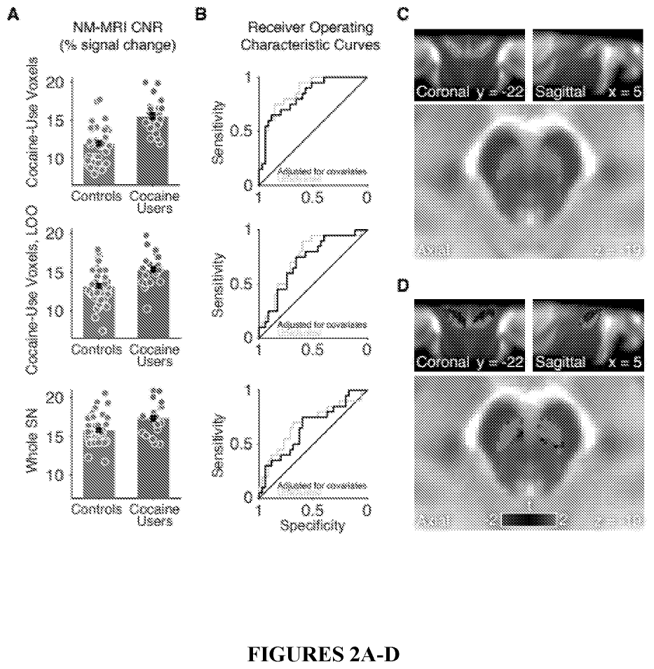

A subset of voxels located mostly ventro-laterally within the SN exhibited significantly increased NM-MRI signal (contrast-to-noise ratio) in cocaine users compared to controls (344 of 1775 voxels at p<0.05, robust linear regression controlling for age, head coil, and cigarettes per day; p_{corrected}=0.025, permutation test; peak voxel MNI coordinates [x, y, z]: 6, −26, −17 mm; see FIG. 2B). In this sample of relatively light smokers, tobacco use was not significantly associated with differences in NM-MRI signal (267 SN voxels exhibited signal that positively correlated with cigarettes per day in the primary linear regression model, p_{corrected}=0.054).

Based on the average NM-MRI signal values extracted from the voxels where cocaine users showed increased NM-MRI signal relative to controls in the voxelwise analysis (cocaine-use voxels, shown in red in FIG. 2B, with extracted values from these voxels shown in FIG. 2A top panel), a diagnosis of cocaine use disorder had a moderate to large effect on NM-MRI signal (Cohen's d=1.34, 95% confidence interval [CI]=0.91-1.90, Cohen's f2=0.46, 95% CI=0.19-0.95; unbiased leave-one-out Cohen's d=0.77, 95% CI=0.35-1.27, Cohen's f2=0.15, 95% CI=0.02-0.43; all estimates based on NM-MRI signal adjusted for age, head coil, and tobacco use). Diagnostic differences in adjusted NM-MRI signal extracted from cocaine-use voxels remained moderate to large when analyzing subsets of the study sample to address possible confounds (controlling for years of education: Cohen's d=0.76, 95% CI=0.22-1.39, n=38; controlling for depressive symptoms: Cohen's d=0.84, 95% CI=0.31-1.52, n=37; controlling for perceived social support: Cohen's d=1.06, 95% CI=0.52-1.72, n=37; excluding non-tobacco users: Cohen's d=1.05, 95% CI=0.50-1.74, n=28; excluding participants scanned with 8-channel coil: Cohen's d=1.38, CI=0.93-1.97, n=51). Furthermore, most cocaine users could be successfully classified relative to all 35 controls based on adjusted NM-MRI signal extracted from cocaine-use voxels (area under the receiver operating characteristic curve [AUC]=0.83, unbiased leave-one-out AUC=0.71; FIG. 2).

For completeness, NM-MRI signal averaged within the whole SN using a region-of-interest analysis was examined. Here again, cocaine users showed significantly increased NM-MRI signal compared to controls (t49=2.07, p=0.044, Cohen's d=0.62, 95% CI=0.19-1.12, robust linear regression controlling for age, head coil, tobacco use, and incomplete SN coverage; AUC=0.69).

Exploratory analysis of the relationship between NM-MRI signal in substantia nigra and measures of cocaine use severity

It was tested whether the NM-MRI signal extracted from cocaine-use voxels correlated with severity of cocaine use and found no significant correlation with duration of use (ρ=−0.33 p=0.18) or money spent on cocaine per week (ρ=−0.08, p=0.74; partial Spearman correlations controlling for age and tobacco use).

Exploratory analysis of the relationship between NM-MRI signal in substantia nigra and ventral striatum response to reward anticipation

To investigate the relationship of the NM-MRI findings to dopamine-related circuit dysfunction in cocaine use disorder, fMRI BOLD activation was measured in the ventral striatum during anticipation of monetary reward. As expected, across all participants, BOLD signal was higher in ventral striatum when anticipating reward compared to no reward (t₃₆=2.56, p=0.015, one-sample t-test of [$5—$0] contrast during anticipation). But this reward-related activation in ventral striatum did not differ between the groups (β=0.038, t₃₂=0.72, p=0.48) or correlate with NM-MRI signal in cocaine use voxels across all participants (β=−0.015, t₃₂=−1.52, p=0.14). There was also no group by NM-MRI signal interaction on reward-related activation in ventral striatum (p=0.24; linear regression controlling for age and tobacco use).

### Discussion

Data is presented herein showing increased NM-MRI signal in the SN of individuals with cocaine use disorder. This increase was not present throughout the whole SN but rather predominated in more ventral and lateral SN subregions. Given that the NM-MRI signal reflects the concentration of synthetic melanins in experimental preparations (8) and of NM in postmortem midbrain tissue (5), and that NM accumulation in SN depends on dopamine function (5, 12, 13), these findings suggest that cocaine users exhibit elevated NM concentration in these SN subregions that may be indicative of dopaminergic dysfunction

The finding of elevated NM signal in cocaine users was surprising given the previous PET studies showing that pre-synaptic dopamine is blunted in cocaine use disorder (1-4). However, this discrepancy provides additional insight into the pathophysiology of dopamine signaling in this disorder. The combination of blunted dopamine release in the striatum with elevated NM in the SN suggests that dopamine is distributed differently in cocaine users compared to controls. Less dopamine concentrated in synaptic vesicles and more dopamine in the cytosolic pool would explain the divergence between PET studies, which estimate dopamine release from vesicles, versus imaging of NM, which accumulates based on the concentration of dopamine in the cytosol (12, 26). If, on the other hand, cocaine use disorder were associated with a global and persistent decrease in dopamine synthesis, a decrease in both the PET and NM-MRI signal would have been expected.

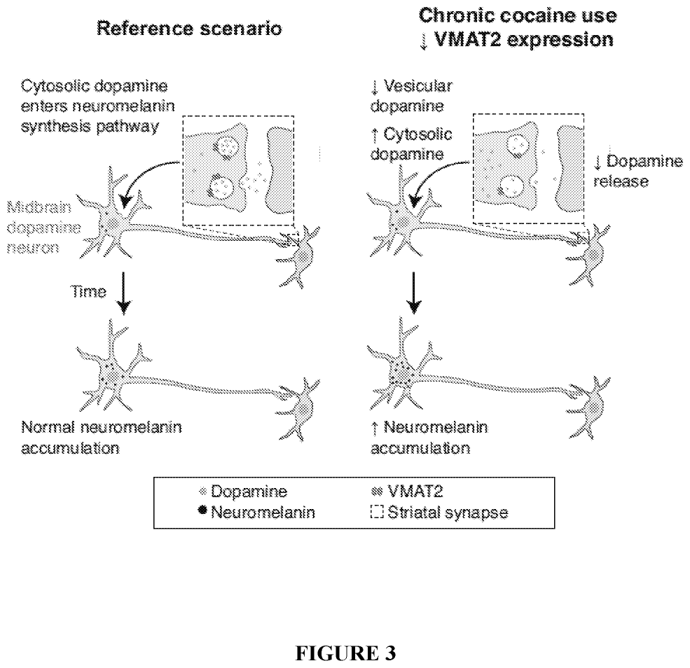

There are a number of previous studies that support the hypothesis that cocaine use disorder involves a redistribution of dopamine between vesicular and cytosolic stores (for graphical depiction of this hypothesis, see FIG. 3). Chronic cocaine exposure is associated with a reduction in vesicular monoamine transporter 2 (VMAT2) expression, which leads to less dopamine in the vesicular pool and more in the cytosolic pool. The reduction in VMAT2 has been shown in nonhuman primates who chronically self-administer cocaine (27) and in human cocaine users (28). Post-mortem human studies also show a reduction of striatal VMAT2 in cocaine users (29-31).

Blunted VMAT2 expression in cocaine use disorder would explain the decrease in pre-synaptic dopamine release seen with PET (1-4) and could also account for the decrease in [18F]DOPA accumulation seen in this population (32), since this likely depends on the radiotracer concentrating in synaptic vesicles (33). Reduced VMAT2 expression has also been shown to correlate with elevated NM formation in the midbrain (12, 34). While cocaine use has been shown to be associated with altered expression of D2 autoreceptors and several other proteins (1, 35), these changes would generally shift both NM accumulation and dopamine release in the same direction. VMAT2 alteration, on the other hand, stands out as a parsimonious explanation for the observed changes occurring in opposing directions. Taken together, these imaging studies suggest that cocaine use is associated with lower dopamine in the vesicular pool and a higher concentration in the cytosolic compartment. However, a study imaging VMAT2 and dopamine release in cocaine users combined with NM-MRI in the midbrain would be needed to confirm the hypothesis. If cocaine use indeed increases cytosolic dopamine, this may pose a risk to neurons because oxidation of dopamine in this compartment forms reactive quinone species (36); however, there is no clear evidence of enhanced dopamine cell death (37) or Parkinson's disease risk (38) in cocaine users.

An alternative interpretation of the main finding is that NM elevation in cocaine users results from repeated episodic surges in dopamine that occurred over the participants' lifetime, which may not be captured by PET. Since NM granules are only removed following cell death (26), and thus serve as a long-term reporter of dopamine function, even a distant history of cocaine use (which may acutely lead to excess dopamine during cocaine consumption) could manifest as a persistent increase in the NM-MRI signal. Future longitudinal studies would be needed to address this possibility.

As an initial test of the functional significance of the findings, it was examined whether NM-MRI signal in cocaine-use voxels within the SN correlated with fMRI response to reward anticipation in the ventral striatum during the Monetary Incentive Delay task, a robust probe of reward system function (15, 19, 25). A significant correlation was not found. This is perhaps unsurprising since the abnormality in cocaine users was not clustered near the “limbic” SN or ventral tegmental area [dorsomedial regions of the over-inclusive SN mask (21)] that send the main projections to ventral striatum. Rather, the topographical analysis showed that group differences predominated in the ventral (or “cognitive”) SN (21), a subregion with prominent projections to the dorsal striatum thought to be involved in cognitive flexibility and other higher-order functions. While PET imaging studies of dopamine function in cocaine users have found consistent evidence of dopaminergic alterations in the dorsal striatum, they have also found pronounced alterations in the ventral striatum. Intriguingly, the observation that cocaine users show an increase in NM-MRI signal in dorsal-striatum-projecting regions of SN but not in ventral-striatum-projecting regions aligns with the previous observation of significant VMAT2 reductions in the dorsal but not the ventral striatum in this population (28, 31). Whatever may underlie this anatomical pattern, it highlights that nigrostriatal circuits sub-serving cognitive functions may be important in cocaine use disorder and that future studies might be better positioned to determine the functional significance of NM-MRI signal change in this disorder by probing higher-order cognitive processes in addition to reward tasks.

The primary limitation of this study is the relatively small, entirely male, sample. However, this first report of NM-MRI in substance use disorders supports the promise of this method for measuring dopamine function in this population. The only previous NM-MRI study to investigate substance use was a preliminary evaluation of the size of the SN area in a small group of patients with psychotic illness. Psychotic patients with comorbid substance use exhibited a larger SN area than non-user patients (39). There is no previous work investigating NM concentration in post-mortem tissue in substance use disorders and this would be an important future direction to provide convergent support for the findings. Further research is needed to address the question of generalization, especially in light of the findings showing a trend-level relationship between NM-MRI and tobacco use (which may well reach significance in a larger sample or in heavier tobacco users). Assuming increased NM signal is due to downregulation of VMAT2 (27, 28), the reported NM-MRI phenotype may be specific to cocaine or other drugs affecting VMAT2 [perhaps including methamphetamine, although its relationship to VMAT2 is less clear (1)]. The absence of significant correlation between NM-MRI signal and duration of cocaine use in the data herein is surprising. Given that NM accumulates over time, it is anticipated that longer duration of use would exaggerate any abnormalities observed in cocaine users. The lack of a significant relationship could, however, be due to the limited range in the duration of use in the sample disclosed herein, as the participants had all been using cocaine for many years. The NM-MRI signal does not reflect a single biological process but could be altered by changes in dopamine synthesis (12), dopamine transfer to vesicles (34), or dopamine cell death (6). Such non-specificity is common to imaging measures (40, 41) and argues for the utility of multimodal studies in triangulating neurobiological mechanisms, as the findings herein can be interpreted in light of previous PET imaging reports. While interpretation of the NM-MRI results is simplified by the absence of enhanced dopamine cell death in cocaine users (37), interpretation of NM-MRI results in disorders showing substantial cell death combined with altered NM accumulation may be more challenging.

Here, NM-MRI evidence has been presented for abnormal NM accumulation in cocaine users, an indirect indication of dopamine dysfunction consistent with prior work. The subject matter disclosed herein thus positions NM-MRI as a promising research tool for addiction and supports its development as a candidate biomarker for stimulant use disorders. Given the central role of dopamine in addiction and the ease of NM-MRI data acquisition, this method has the potential to advance the understanding of dopamine alterations in addiction, particularly as it affords the opportunity to study younger, at-risk populations and describe longitudinal trajectories of dopamine alterations, which have been challenging to study using PET.

### REFERENCES FOR EXAMPLE 3

1. Ashok A H, Mizuno Y, Volkow N D, Howes O D. Association of Stimulant Use With Dopaminergic Alterations in Users of Cocaine, Amphetamine, or Methamphetamine: A Systematic Review and Meta-analysis. JAMA Psychiatry. 2017; 74:511-519.
2. Martinez D, Carpenter K M, Liu F, Slifstein M, Broft A, Friedman A C, Kumar D, Van Heertum R, Kleber H D, Nunes E. Imaging dopamine transmission in cocaine dependence: link between neurochemistry and response to treatment. Am J Psychiatry. 2011; 168:634-641.
3. Martinez D, Narendran R, Foltin R W, Slifstein M, Hwang D R, Broft A, Huang Y, Cooper T B, Fischman M W, Kleber H D, Laruelle M. Amphetamine-induced dopamine release: markedly blunted in cocaine dependence and predictive of the choice to self-administer cocaine. Am J Psychiatry. 2007; 164:622-629.
4. Volkow N D, Wang G J, Fowler J S, Logan J, Gatley S J, Hitzemann R, Chen A D, Dewey S L,
Pappas N. Decreased striatal dopaminergic responsiveness in detoxified cocaine-dependent subjects. Nature. 1997; 386:830-833.
5. Cassidy C M, Zucca F A, Girgis R R, Baker S C, Weinstein J J, Sharp M E, Bellei C, Valmadre A, Vanegas N, Kegeles L S, Brucato G, Jung Kang U, Sulzer D, Zecca L, Abi-Dargham A, Horga G. Neuromelanin-sensitive MRI as a noninvasive proxy measure of dopamine function in the human brain. Proc Natl Acad Sci USA. 2019; 116:5108-5117.
6. Sulzer D, Cassidy C, Horga G, Kang U J, Fahn S, Casella L, Pezzoli G, Langley J, Hu X P, Zucca F A, Isaias I U, Zecca L. Neuromelanin detection by magnetic resonance imaging (MRI) and its promise as a biomarker for Parkinson's disease. NPJ Parkinsons Dis. 2018; 4:11.
7. Zecca L, Fariello R, Riederer P, Sulzer D, Gatti A, Tampellini D. The absolute concentration of nigral neuromelanin, assayed by a new sensitive method, increases throughout the life and is dramatically decreased in Parkinson's disease. FEBS Lett. 2002; 510:216-220.
8. Trujillo P, Summers P E, Ferrari E, Zucca F A, Sturini M, Mainardi L T, Cerutti S, Smith A K, Smith S A, Zecca L, Costa A. Contrast mechanisms associated with neuromelanin-MRI. Magn Reson Med. 2017; 78:1790-1800.
9. Zecca L, Bellei C, Costi P, Albertini A, Monzani E, Casella L, Gallorini M, Bergamaschi L, Moscatelli A, Turro N J, Eisner M, Crippa P R, Ito S, Wakamatsu K, Bush W D, Ward W C, Simon J D, Zucca F A. New melanic pigments in the human brain that accumulate in aging and block environmental toxic metals. Proc Natl Acad Sci USA. 2008; 105:17567-17572.
10. Martin-Bastida A, Lao-Kaim N P, Roussakis A A, Searle G E, Xing Y, Gunn R N, Schwarz S T, Barker R A, Auer D P, Piccini P. Relationship between neuromelanin and dopamine terminals within the Parkinson's nigrostriatal system. Brain. 2019; 142:2023-2036.
11. Watanabe Y, Tanaka H, Tsukabe A, Kunitomi Y, Nishizawa M, Hashimoto R, Yamamori H, Fujimoto M, Fukunaga M, Tomiyama N. Neuromelanin magnetic resonance imaging reveals increased dopaminergic neuron activity in the substantia nigra of patients with schizophrenia. PLoS One. 2014; 9:e104619.
12. Sulzer D, Bogulavsky J, Larsen K E, Behr G, Karatekin E, Kleinman M H, Turro N, Krantz D, Edwards R H, Greene L A, Zecca L. Neuromelanin biosynthesis is driven by excess cytosolic catecholamines not accumulated by synaptic vesicles. Proc Natl Acad Sci USA. 2000; 97:11869-11874.
13. Cebrian C, Zucca F A, Mauri P, Steinbeck J A, Studer L, Scherzer C R, Kanter E, Budhu S, Mandelbaum J, Vonsattel J P, Zecca L, Loike J D, Sulzer D. MHC-I expression renders catecholaminergic neurons susceptible to T-cell-mediated degeneration. Nat Commun. 2014; 5:3633.
14. McCutcheon R A, Abi-Dargham A, Howes O D. Schizophrenia, Dopamine and the Striatum: From Biology to Symptoms. Trends Neurosci. 2019; 42:205-220.
15. Oldham S, Murawski C, Fornito A, Youssef G, Yucel M, Lorenzetti V. The anticipation and outcome phases of reward and loss processing: A neuroimaging meta-analysis of the monetary incentive delay task. Hum Brain Mapp. 2018; 39:3398-3418.
16. Urban N B, Slifstein M, Meda S, Xu X, Ayoub R, Medina O, Pearlson G D, Krystal J H, Abi-Dargham A. Imaging human reward processing with positron emission tomography and functional magnetic resonance imaging. Psychopharmacology (Berl). 2012; 221:67-77.
17. Schott B H, Minuzzi L, Krebs R M, Elmenhorst D, Lang M, Winz O H, Seidenbecher C I, Coenen H H, Heinze H J, Zilles K, Duzel E, Bauer A. Mesolimbic functional magnetic resonance imaging activations during reward anticipation correlate with reward-related ventral striatal dopamine release. J Neurosci. 2008; 28:14311-14319.
18. Luijten M, Schellekens A F, Kuhn S, Machielse M W, Sescousse G. Disruption of Reward Processing in Addiction: An Image-Based Meta-analysis of Functional Magnetic Resonance Imaging Studies. JAMA Psychiatry. 2017; 74:387-398.
19. Balodis I M, Potenza M N. Anticipatory reward processing in addicted populations: a focus on the monetary incentive delay task. Biol Psychiatry. 2015; 77:434-444.
20. Weinstein J J, Chohan M O, Slifstein M, Kegeles L S, Moore H, Abi-Dargham A. Pathway-Specific Dopamine Abnormalities in Schizophrenia. Biol Psychiatry. 2017; 81:31-42.
21. Zhang Y, Larcher K M, Misic B, Dagher A. Anatomical and functional organization of the human substantia nigra and its connections. Elife. 2017; 6.
22. Zimet G D, Dahlem N W, Zimet S G, Farley G K. The multidimensional scale of perceived social support. Journal of Personality Assessment. 1988; 52:30-41.
23. Beck A T, Steer R A, Ball R, Ranieri W. Comparison of Beck Depression Inventories-IA and -II in psychiatric outpatients. J Pers Assess. 1996; 67:588-597.
24. Andrews M M, Meda S A, Thomas A D, Potenza M N, Krystal J H, Worhunsky P, Stevens M C, O'Malley S, Book G A, Reynolds B, Pearlson G D. Individuals family history positive for alcoholism show functional magnetic resonance imaging differences in reward sensitivity that are related to impulsivity factors. Biol Psychiatry. 2011; 69:675-683.
25. Wu C C, Samanez-Larkin G R, Katovich K, Knutson B. Affective traits link to reliable neural markers of incentive anticipation. Neuroimage. 2014; 84:279-289.
26. Zucca F A, Basso E, Cupaioli F A, Ferrari E, Sulzer D, Casella L, Zecca L. Neuromelanin of the human substantia nigra: an update. Neurotox Res. 2014; 25:13-23.
27. Narendran R, Jedema H P, Lopresti B J, Mason N S, Himes M L, Bradberry C W. Decreased vesicular monoamine transporter type 2 availability in the striatum following chronic cocaine self-administration in nonhuman primates. Biol Psychiatry. 2015; 77:488-492.
28. Narendran R, Lopresti B J, Martinez D, Mason N S, Himes M, May M A, Daley D C, Price J C, Mathis C A, Frankle W G. In vivo evidence for low striatal vesicular monoamine transporter 2 (VMAT2) availability in cocaine abusers. Am J Psychiatry. 2012; 169:55-63.
29. Little K Y, Krolewski D M, Zhang L, Cassin B J. Loss of striatal vesicular monoamine transporter protein (VMAT2) in human cocaine users. Am J Psychiatry. 2003; 160:47-55.
30. Little K Y, Zhang L, Desmond T, Frey K A, Dalack G W, Cassin B J. Striatal dopaminergic abnormalities in human cocaine users. Am J Psychiatry. 1999; 156:238-245.
31. Wilson J M, Levey Al, Bergeron C, Kalasinsky K, Ang L, Peretti F, Adams V I, Smialek J, Anderson W R, Shannak K, Deck J, Niznik H B, Kish S J. Striatal dopamine, dopamine transporter, and vesicular monoamine transporter in chronic cocaine users. Ann Neurol. 1996; 40:428-439.
32. Wu J C, Bell K, Najafi A, Widmark C, Keator D, Tang C, Klein E, Bunney B G, Fallon J, Bunney W E. Decreasing striatal 6-FDOPA uptake with increasing duration of cocaine withdrawal. Neuropsychopharmacology. 1997; 17:402-409.
33. Kumakura Y, Cumming P. PET studies of cerebral levodopa metabolism: a review of clinical findings and modeling approaches. Neuroscientist. 2009; 15:635-650.
34. Liang C L, Nelson O, Yazdani U, Pasbakhsh P, German D C. Inverse relationship between the contents of neuromelanin pigment and the vesicular monoamine transporter-2: human midbrain dopamine neurons. J Comp Neurol. 2004; 473:97-106.
35. Worhunsky P D, Matuskey D, Gallezot J D, Gaiser E C, Nabulsi N, Angarita G A, Calhoun V D, Malison R T, Potenza M N, Carson R E. Regional and source-based patterns of [(11)C]-(+)-PHNO binding potential reveal concurrent alterations in dopamine D2 and D3 receptor availability in cocaine-use disorder. Neuroimage. 2017; 148:343-351.
36. Segura-Aguilar J, Paris I, Munoz P, Ferrari E, Zecca L, Zucca F A. Protective and toxic roles of dopamine in Parkinson's disease. J Neurochem. 2014; 129:898-915.
37. Bennett B A, Hyde C E, Pecora J R, Clodfelter J E. Long-term cocaine administration is not neurotoxic to cultured fetal mesencephalic dopamine neurons. Neurosci Lett. 1993; 153:210-214.
38. Asser A, Taba P. Psychostimulants and movement disorders. Front Neurol. 2015; 6:75.
39. Tavares M, Reimao S, Chendo I, Carvalho M, Levy P, Nunes R G. Neuromelanin magnetic resonance imaging of the substantia nigra in first episode psychosis patients consumers of illicit substances. Schizophr Res. 2018; 197:620-621.
40. Guo N, Hwang D R, Lo E S, Huang Y Y, Laruelle M, Abi-Dargham A. Dopamine depletion and in vivo binding of PET D1 receptor radioligands: implications for imaging studies in schizophrenia. Neuropsychopharmacology. 2003; 28:1703-1711.
41. Logothetis N K. What we can do and what we cannot do with fMRI. Nature. 2008; 453:869-878.

### Example 4—Association Between Neuromelanin-Sensitive MRI Signal and Psychomotor Slowing in Late-Life Depression

Abstract

Late-life depression (LLD) is a prevalent and disabling condition in older adults that is often accompanied by slowed processing and gait speed. These symptoms are related to impaired dopamine function and sometimes remedied by levodopa (L-DOPA). In this study, 33 older adults with LLD were recruited to determine the association between a proxy measure of dopamine function-neuromelanin-sensitive magnetic resonance imaging (NM-MRI)—and baseline slowing measured by the Digit Symbol test and a gait speed paradigm. In secondary analyses, the ability of NM-MRI to predict L-DOPA treatment response in a subset of these patients (N=15) who received 3 weeks of L-DOPA was also assessed. A further subset of these patients (N=6) were scanned with NM-MRI at baseline and after treatment to evaluate the effects of L-DOPA treatment on the NM-MRI signal. It was found that lower baseline NM-MRI correlated with slower baseline gait speed (346 of 1,807 substantia nigra-ventral tegmental area (SN-VTA) voxels, P_{corrected}=0.038), particularly in the more medial, anterior, and dorsal SN-VTA. Secondary analyses failed to show an association between baseline NM-MRI and treatment-related changes in gait speed, processing speed, or depression severity (all P_{corrected}>0.361); evidence of increases in the NM-MRI signal 3 weeks post-treatment with L-DOPA compared to baseline was found (200 of 1,807 SN-VTA voxels; P_{corrected}=0.046). Overall, the findings indicate that NM-MRI is sensitive to variability in gait speed in patients with LDD, suggesting this non-invasive MRI measure may provide a promising marker for dopamine-related psychomotor slowing in geriatric neuropsychiatry.

### Introduction

Late life depression (LLD) is a prevalent and disabling condition among older adults that is often recurrent, can become chronic, and is frequently non-responsive to antidepressant medication (1-4). Motivational deficits, slowed processing speed, and gait impairments are prominent aspects of the LLD phenotype and suggest dopaminergic dysfunction may play a key pathophysiologic role (5-7). These features are negative prognostic factors for antidepressant treatment (8) and more broadly portend adverse health outcomes, including death (9, 10). Recent work suggests that carbidopa/levodopa (L-DOPA) monotherapy significantly improves processing speed, gait speed, and depressive symptoms in depressed older adults by increasing dopamine availability in selected striatal subregions (11). However, LLD is a heterogeneous and etiologically complex disorder, suggesting the need for non-invasive and scalable methods to identify dopamine-deficient individuals and personalize their treatment. As a first step in this direction, here the ability of neuromelanin-sensitive magnetic resonance imaging (NM-MRI) to capture dopamine-related phenotypes in LDD was tested, particularly psychomotor slowing.

Psychomotor slowing is of great clinical importance to LDD and it has been linked to dopamine function. In LLD, decreased processing speed predicts poorer acute response to antidepressants (8) and higher risk for dementia (12), while slowed gait increases the risk of falls (13), disability (14), and mortality (6). Psychomotor slowing in older individuals is thought to stem at least in part from decreases in dopamine transmission with aging (15-17), consistent with human and preclinical work linking mesostriatal dopaminergic transmission to gait speed (18, 19). Given this link, the presence of psychomotor slowing may indicate an underlying dopaminergic deficit that could be central to the pathophysiology of LDD (7), and which could possibly be remediated via pro-dopaminergic treatments such as L-DOPA. Indeed, previous work showed that, in LLD individuals with slowed gait speed, L-DOPA monotherapy can ameliorate psychomotor slowing and depressive symptoms by normalizing mesostriatal dopamine transmission (11). While these results are encouraging, slowed gait speed is an indirect and unspecific marker of dopamine deficits, suggesting that more direct measures like NM-MRI could optimize the selection of LDD patients who may benefit most from L-DOPA treatment.

NM-MRI is a noninvasive imaging technique that enables visualization of neuromelanin (NM) concentration in NM-rich regions (20, 21). NM is a product of dopamine metabolism that accumulates in the dopaminergic neurons of the substantia nigra (SN) (22-25). NM-MRI imaging of the SN was recently validated as a marker of dopamine function, with the NM-MRI signal correlating with positron emission tomography (PET) measures of dopamine release capacity in the striatum, and capturing dopamine dysfunctions associated with psychiatric illness (20). NM-MRI is therefore uniquely suited as a potential biomarker for treatment selection in patients with dopamine dysfunction, including at least some LDD patients, and one that could be broadly adopted given its non-invasiveness, cost-effectiveness, and lack of ionizing radiation.

The goal of the present study was to determine the suitability of NM-MRI as a potential biomarker for psychomotor slowing and to begin testing its ability to predict and monitor of L-DOPA treatment response in LLD. Without being bound by theory, it is thought that individuals with slower processing and those with slower gait would exhibit lower dopamine function as measured by NM-MRI. Furthermore, in a secondary analysis in a small sample, the ability of NM-MRI to predict the improvement of psychomotor slowing after L-DOPA treatment was investigated. In an analysis in a further subset of patients, the sensitivity of NM-MRI to capture longitudinal changes in dopamine function associated with L-DOPA treatment was also investigated.

Methods and Materials

Subjects

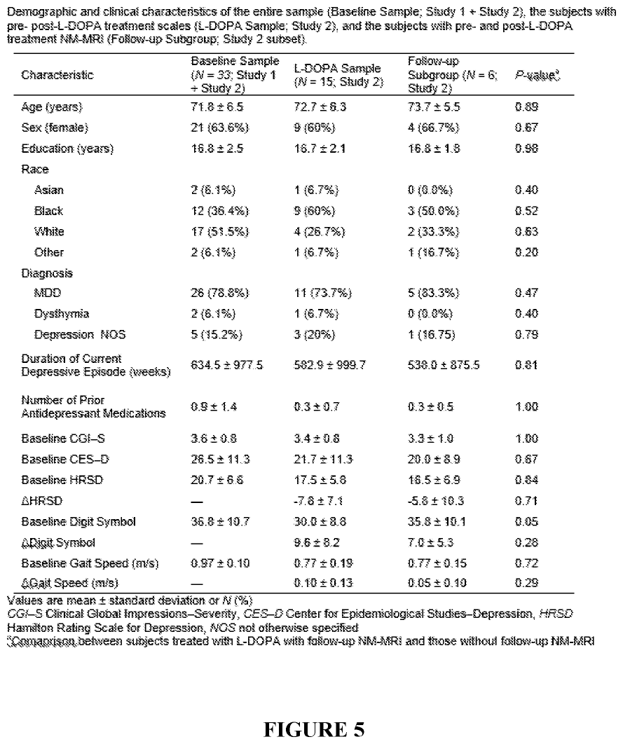

The studies described were conducted in the Adult and Late Life Depression Research Clinic at the New York State Psychiatric Institute (NYSPI) and were approved by the NYSPI Institutional Review Board. The research program on LLD encompasses numerous therapeutic and pathophysiologic studies. In order to increase the sample size, data was aggregated from two studies having similar selection criteria and utilizing the same NM-MRI sequence. The first study (N=18; Study 1) was an antidepressant treatment trial, from which only the baseline data was used. A second study (N=15; Study 2) was an open-label L-DOPA trial, from which the baseline and post-treatment data was used (pre-post L-DOPA dataset). Of these 15 individuals, follow-up NM-MRI data after receiving L-DOPA was collected in 6. See FIG. 5 for further depiction of the sample included in the analyses. All subjects (N=33; Study 1+Study 2) were adult outpatients aged ≥60 years who were diagnosed with Diagnostic and Statistical Manual 5 major depressive disorder, dysthymia, or depression not otherwise specified, and had a minimum depressive symptom score on a standardized scale (Hamilton Rating Scale for Depression [HRSD]≥16 or Center for Epidemiologic Studies-Depression Rating Scale ≥10). Subjects who exhibited substance abuse or dependence, were diagnosed with a psychotic disorder, bipolar disorder, or probable dementia, had a Mini Mental Status Examination score ≤24, an HRSD suicide item>2, or a Clinical Global Impressions-Severity score of 7 at baseline were all excluded. Subjects with an acute or severe medical illness, mobility limiting osteoarthritis or joint disease, a contraindication to MRI, or who had been treated within the past 4 weeks with psychotropic or other medications known to affect dopamine were excluded as well.

Assessments

Processing speed was assessed using the Digit Symbol test from the Wechsler Adult Intelligence Scale-III (26). Gait speed was measured in m/s as a single task in which study participants walked at their usual or normal speed on a 15-foot walking course. Two trials were completed, and the final gait speed measurement was recorded as the average of these two trials. Depression severity was assessed using the 24-item HRSD.

Study 1 Design

Assessments and MRI data were obtained at baseline, prior to beginning antidepressant treatment (N=18). Further details can be found at clinicaltrials.gov/ct2/show/NCT01931202.

Study 2 Design

Inclusion in this study also required decreased gait speed (defined as average walking speed over 15′ course <1 m/s). Assessments and MRI data were obtained at baseline, prior to beginning L-DOPA treatment (N=15). After their MRI scan, subjects began taking 37.5 mg carbidopa/150 mg levodopa once daily (9 am). After one week at this dosage, subjects were instructed to take 37.5 mg carbidopa/150 mg levodopa twice daily (9 am and 5 pm). For the third week of treatment, subjects took 37.5 mg carbidopa/150 mg levodopa three times daily (9 am, 12 pm, and 5 pm). Participants were instructed to maintain the same timing of doses throughout the study as described above. A subset of these participants (N=6) had a post-treatment MRI scan after a Week 3 visit when post-treatment assessments were performed. Please refer to the previously published main outcome manuscript for a full description of study procedures (11); further details can be found at clinicaltrials.gov/ct2/show/NCT02744391. Processing and gait speed were assessed at baseline and then weekly during L-DOPA treatment (i.e., Weeks 0-3). Assessments were performed at approximately 1 pm to control for time of day effects and the duration since the last morning L-DOPA dose (anticipated to be 4 hours). HRSD was also performed at Week 0 and Week 3. Changes in processing speed, gait speed, and HRSD were taken as the difference between Week 3 and Week 0.

Magnetic Resonance Imaging

Magnetic resonance images of the brain were acquired for all participants on a GE MR750 3.0 T scanner using a 32-channel phased-array Nova head-coil. NM-MRI data were acquired with a 2D gradient-recalled echo sequence with magnetization transfer contrast (2D GRE-MT) with the following parameters (20): repetition time (TR)=260 ms; echo time (TE)=2.68 ms; flip angle=40°; in-plane resolution=0.39×0.39 mm2; partial brain coverage with field of view (FoV)=162×200; matrix=416×512; number of slices=10; slice thickness=3 mm; slice gap=0 mm; magnetization transfer frequency offset=1,200 Hz; number of excitations (NEX)=8; acquisition time=8.04 min. The slice-prescription protocol consisted of orienting the image stack along the anterior-commissure-posterior-commissure line and placing the top slice 3 mm below the floor of the third ventricle, viewed on a sagittal plane in the middle of the brain. This protocol provided coverage of SN-containing portions of the midbrain (and cortical and subcortical structures surrounding the brainstem) with high in-plane spatial resolution using a short scan easy to tolerate by clinical populations. For preprocessing of the NM-MRI data, a whole-brain, high-resolution T1-weighted 3D BRAVO structural MRI scan was acquired with the following parameters: inversion time=450 ms, TR=7.85 ms, TE=3.10 ms, flip angle=12°, FoV=240×240, matrix=300×300, number of slices=220, isotropic voxel size=0.8 mm3).

NM-MRI data were preprocessed using a pipeline combing SPM and ANTs, previously shown to achieve high test-retest reliability (27). The pipeline consisted of the following steps: (1) brain extraction of the T1w image using ‘antsBrainExtraction.sh’; (2) spatial normalization of the brain-extracted T1w image to MNI space using ‘antsRegistrationSyN.sh’ (rigid+affine+deformable syn); (3) coregistration of the NM-MRI image to the T1w image using ‘antsRegistrationSyN.sh’ (rigid); (4) spatial normalization of the NM-MRI images to MNI space by a single-step transformation combing the transformations estimated in steps (2) and (3) using ‘antsApplyTransforms’; (5) resampling of the spatially-normalized NM-MRI image to 1 mm isotropic resolution using ‘ResampleImage’; (6) spatial smoothing of the spatially-normalized NM-MRI image with a 1 mm full-width-at-half-maximum Gaussian kernel using ‘SPM-Smooth’. The preprocessed NM-MRI images were then used to estimate NM-MRI contrast ratio (CNR) maps. NM-MRI CNR at each voxel was calculated as the percent signal difference in NM-MRI signal intensity at a given voxel (IV) from the signal intensity in the crus cerebri (ICC), a region of white matter tracts known to have minimal NM content as: CNR_{V}={[I_{V}−mode(I_{CC})]/mode(I_{CC})}*100. Where mode(ICC) was calculated for each participant from a kernel-smoothing-function fit of a histogram of all voxels in the CC mask (20).

Statistical Analysis

The a priori analysis tested the hypothesis that lower baseline NM-MRI CNR would correlate with slower psychomotor variables (Digit Symbol and gait speed; N=33; Study 1+Study 2). In a secondary analysis we investigated if baseline NM-MRI CNR would predict L-DOPA-induced improvements (speeding) of these psychomotor variables (N=15; Study 2). These effects were tested within the substantia nigra-ventral tegmental area (SN-VTA) complex using a voxelwise analysis approach validated in Cassidy et al. (20). Briefly, this method uses robust linear regression analyses and tests for significance of regression coefficients using permutation tests. The linear model used to test the a priori hypothesis (model 1) was: CNR_{V}=β₀+β₁·gait speed+β₂·Digit Symbol score+β₃·HRSD+β₄·age+β₅·gender+β₆·education, with β₁₋₃ being the variables of interest and β₄₋₆ covariates of no-interest. The linear model for the secondary analysis (model 2) was: CNR_{V}=β₀+β₁·Δgait speed+β₂·ΔDigit Symbol score+β₃·ΔHRSD+β₄·gait speed+β₅·Digit Symbol score+β₆·HRSD+β₇·age+β₈·gender+β₉·education, with β₁₋₃ being the variables of interest and β₄₋₉ covariates of no-interest. The inclusion of all variables of interest in one model provides greater specificity of effects while also providing a more conservative test that guards against false positives by adjusting the degrees of freedom in t-tests of regression coefficients (28). The number of voxels showing a significant effect was determined to be significant through permutation testing, wherein 10,000 iterations of random permutations of the variables of interest were run while keeping the covariates of no-interest constant—see Cassidy et al. for further details (20). This voxelwise permutation-test corrects for multiple comparisons across voxels and provides adequate protection against false positives, similar to methods used in functional-MRI studies (29).

In an exploratory analysis, we also investigated if changes in NM-MRI CNR can be detected after 3 weeks of L-DOPA treatment (N=6; subset from Study 2). A similar voxelwise analysis approach was used, except it used a non-parametric, sign-rank test comparing pre- and post-L-DOPA treatment NM-MRI CNR values. The number of voxels showing a significant effect was determined to be significant through a permutation test in which the null distribution was derived by 10,000 iterations of random assignment of the pre- and post-L-DOPA treatment labels for each subject (i.e., 50% chance for a subject's pre-L-DOPA treatment NM-MRI CNR value to be assigned as their post-L-DOPA treatment value, with their post-L-DOPA treatment value being assigned as their pre-L-DOPA treatment value).

A priori power analyses using effect sizes comparing baseline gait speed and dopamine function measure by PET (19) demonstrated 85% power to detect an effect in the baseline sample of 33 subjects (two-tailed, □=0.05) but only 50% power in the L-DOPA sample of 15 subjects. Thus, the analyses in the former sample (model 1) were sufficiently powered as the a priori test. No additional corrections were implemented across a priori and secondary tests given the exploratory nature of the latter, which are presented for completeness and descriptive purposes.

To rule out potential selection bias in the follow-up NM-MRI subset from Study 2, Pearson chi-square tests or Mann-Whitney U tests were used to compare demographic and clinical characteristics between the participants in Study 2 who either received a follow-up NM-MRI scan after 3 weeks of L-DOPA treatment (N=6) and those who did not receive a follow-up NM-MRI scan after treatment (N=9).

Results

Sample Characteristics

Clinical and demographic characteristics of the sample are provided in FIG. 5; for all 33 subjects, mean age was 71.8±6.5 years, 63.6% were female, mean education was 16.8±2.5 years, mean gait speed was 0.97±0.32 m/s, mean Digit Symbol score was 36.8±10.7, and mean HRSD was 20.7±6.6. N; significant differences were observed between subjects in Study 2 with a follow-up NM-MRI scan and those without a follow-up NM-MRI scan.

Baseline Gait Speed is Associated with Baseline NM-MRI

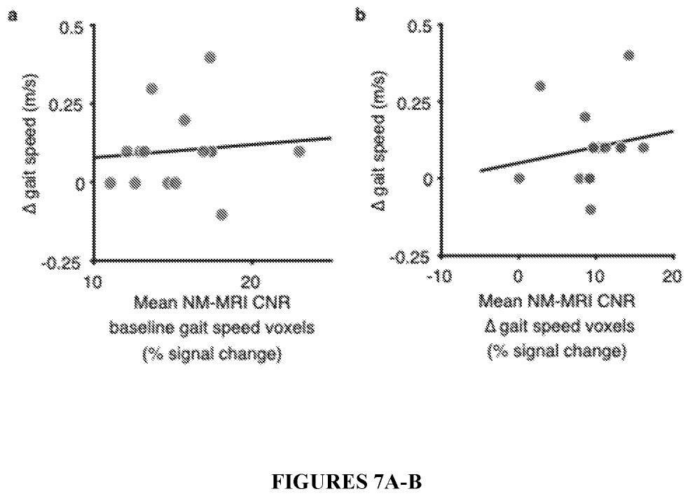

Without being bound by theory, an a priori hypothesis was investigated that individuals with slower processing and those with slower gait would exhibit lower dopamine function as measured by NM-MRI in 33 patients with LLD (Study 1+Study 2). A voxelwise linear regression model (model 1) predicted NM-MRI CNR within the SN-VTA mask as a function of gait speed, Digit Symbol score, and HRSD, with age, gender, and education as covariates. This revealed a set of SN-VTA voxels in which NM-MRI CNR correlated positively with gait speed (346 of 1,807 SN-VTA voxels at P<0.05, robust linear regression; P_{corrected}=0.038, permutation test; FIG. 7). In contrast, there was no significant effect for Digit Symbol score (194 of 1,807 SN-VTA voxels at P<0.05; P_{corrected}=0.121, permutation test) or HRSD (19 of 1,807 SN-VTA voxels at P<0.05; P_{corrected}=0.731, permutation test). A topographical analysis of the relationship between gait speed and NM-MRI CNR showed stronger relationships tended to occur in more medial (□_{|x|}=0.02, t₁₈₀₃=2.40, P=0.016), anterior (□_{y}=0.14, t₁₈₀₃=25.8, P=10⁻¹²⁴), and dorsal (□_{z}=−0.05, t₁₈₀₃=−6.62, P=10⁻¹⁰) SN-VTA voxels [multiple linear regression analysis predicting t statistic of gait speed effect across SN-VTA voxels as a function of their coordinates in x (absolute distance from the midline), y, and z directions: omnibus F_{3,1803}=297, P=10⁻¹⁵⁵].

Secondary Analyses Fail to Show Associations Between Baseline NM-MRI and Changes in Psychomotor Speed with L-DOPA Treatment

In a secondary analysis, the relationship between baseline NM-MRI signal and changes in psychomotor speed after 3 weeks of L-DOPA treatment in 15 patients with both baseline and post-treatment psychomotor evaluations (Study 2) was investigated. As a more stringent and spatially constrained test of this relationship, it was first determined if there was a relationship between changes in gait speed after 3 weeks of L-DOPA treatment and the average NM-MRI CNR in the 346 SN-VTA voxels that correlated positively with baseline gait speed (green voxels in FIG. 1). Here, were found no relationship between baseline NM-MRI CNR and the change in gait speed (t_{1,9}=0.71, P=0.49; robust linear regression testing for the effect of change in gait speed adjusting for baseline gait speed, age, gender, and education; FIG. 7). As a more lenient test of the hypothesis, a voxelwise analysis was performed in which, for each subject, the relationship was investigated between changes in gait speed and Digit Symbol scores after L-DOPA treatment with baseline NM-MRI CNR within the SN-VTA mask at each voxel (model 2). Again, no relationship was found between baseline NM-MRI CNR and the change in gait speed (64 of 1,807 SN-VTA voxels at P<0.05, robust linear regression testing for the effects of change in gait speed, change in Digit Symbol score, and change in HRSD adjusting for baseline gait speed, baseline Digit Symbol score, baseline HRSD age, gender, and education; P_{corrected}=0.377, permutation test), change in Digit Symbol score (69 of 1,807 SN-VTA voxels at P<0.05, P_{corrected}=0.361, permutation test), or change in HRSD (67 of 1,807 SN-VTA voxels at P<0.05, P_{corrected}=0.371, permutation test).

Increases in NM-MRI CNR in the SN-VTA with L-DOPA Treatment

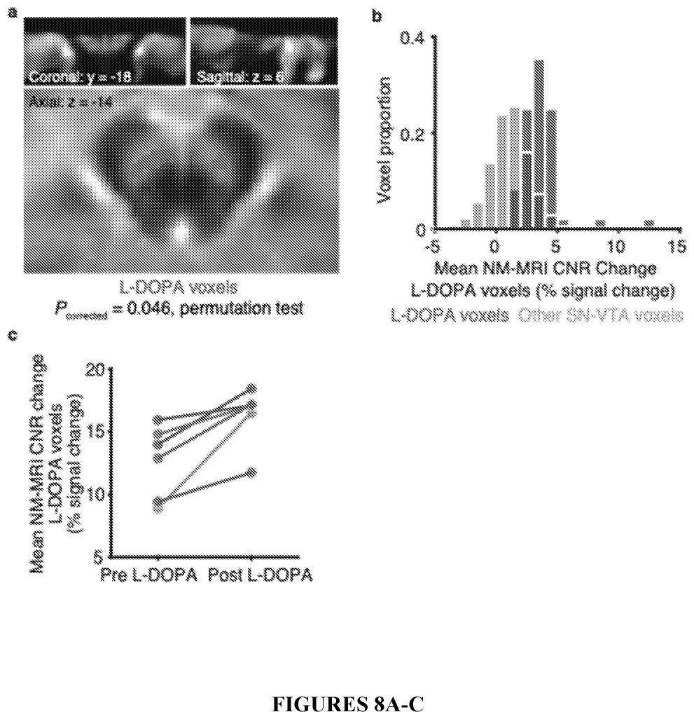

In an exploratory analysis, it was also investigated whether the NM-MRI signal changed after 3 weeks of L-DOPA treatment in the 6 patients with available baseline and post-treatment MRI data (Study 2 subset). To this end, a non-parametric voxelwise analysis was performed in which, for each subject, the difference in NM-MRI CNR at baseline and post-treatment within the SN-VTA mask at each voxel was tested. This revealed a set of SN-VTA voxels where NM-MRI CNR was significantly higher in the post-treatment scans (200 of 1,807 SN-VTA voxels at P<0.05, sign-rank test testing for the difference in NM-MRI CNR at baseline and post-treatment; P_{corrected}=0.046, permutation test; FIG. 8).

### Discussion

As a diagnostic biomarker for the symptoms of PD: determination of symptoms of a motor disorder and prediction of severity of current symptoms

The voxel based analysis of NM-MRI includes voxels used to determine neuromelanin concentrations, voxels used to determine neuromelanin volumes, and certain voxels associated with specific symptoms (termed symptom-specific voxels and in this case motor disorder symptom voxels, for example, psychomotor slowing voxels). The symptom of psychomotor slowing has been shown to occur in Parkinson's disease and the voxel based analysis method may function as a diagnostic biomarkers and also determine the severity of specific symptoms

In Parkinson's disease (PD), gait speed is significantly slowed (Peterson et al 2020). First, the relationship was investigated between NM-MRI data and psychomotor speed in older adults with late life depression (LLD) and found that lower NM-MRI signal in medial, anterior and dorsal parts of the SN-VTA complex, as determined by the voxel based analysis method, was associated with slower gait speed. This provides evidence that both the presence and severity of a key motor symptom of PD (in this example psychomotor slowing) can be predicted by the voxel based analysis of neuromelanin MRI data providing a noninvasive method to determine important information that may guide care of these symptoms.

The finding of lower dopamine function, as indexed by lower NM-MRI signal, being associated with slower gait speed is consistent with the a priori hypotheses based on previous literature. For example, recent studies have identified a relationship between a genetic polymorphism of Catechol-O-methyltransferase (COMT, rs4680; which regulates tonic dopamine) and gait speed (30, 31). Additionally, in older patients with cerebral small vessel disease, gait decline has been attributed to reductions in nigrostriatal dopamine. More generally, a strong theoretical foundation implicating dopamine function of the dorsal basal ganglia in age-related motor dysfunction has been proposed and supports the need for dopaminergic biomarkers in this area.

This data shows that the baseline NM-MRI data is able to predict the symptom of psychomotor speed. Since neuromelanin is reduced in PD, and gait slowing is a hallmark, this analysis method may be able to predict specific symptoms of PD with specific voxels. The prediction of different symptoms on NM-MRI could provide a non-invasive way to determine a diagnosis of Parkinson's disease and differentiate from related disorders with different motor symptoms.

The finding that dopamine function indexed by NM-MRI signal was associated with a nonsignificant trend with Digit Symbol scores was limited by the small sample size (N=33) which restricts the ability to determine a significant association between Digit Symbol scores and dopamine function, and studies in larger samples are required to address this. Dopamine is theoretically linked to processing speed, but empirical evidence correlating neuroimaging-based measures of dopamine signaling with performance on processing speed tasks is mixed. The largest study to date (N=181 healthy adults) showed no significant correlation between striatal raclopride PET D2-receptor binding and processing speed; although smaller studies have observed small, but significant, associations between processing speed and dopamine function. The Applicant is not aware of any studies to have demonstrated significant correlations between dopamine signaling and Digit Symbol scores. Thus, while the Digit Symbol test's motor requirements and speed dependence is theoretically suggestive of a link to dopamine function, there may be more complexity involved. Furthermore, although motor speed and attention are impaired in both aging and depressed populations, these deficits are often subtle and not detected through the Digit Symbol test; and the mechanisms for their impairment in these clinical populations may not be dopaminergic.

As a diagnostic biomarker for the diagnosis of PD and to rule out related disorders

The data supports the ability of the voxel based analysis method to differentiate LLD from Parkinson's disease based on NM-MRI. For example, the results of the topographical analysis of the relationship between gait speed and NM-MRI signal showed that stronger relationships occurred in the medial, anterior, and dorsal areas of the SN-VTA. In contrast, NM-MRI data have shown that larger signal decreases in PD tend to predominate in more lateral, posterior and ventral voxels. Furthermore, histopathological studies have also found that PD-related neuron loss occurs mainly in the ventrolateral tier of the SN, with recent free water imaging studies identifying similar spatial patterns. A recent study used NM-MRI to analyze the signal intensity of the SN in two motor subtypes of PD, with patients classified as either postural instability, gait difficulty dominant or tremor dominant, along with controls. Significant signal attenuation was detected in the lateral part of SN in both PD subtypes when compared with the controls, and severe signal attenuation was also observed in the medial part of SN in postural instability, gait difficulty dominant patients in comparison with the tremor dominant group (52). Taken together, the topological findings, in addition to the fact that slowed, depressed subjects typically do not manifest the clinical stigmata of PD (e.g., cog wheeling, freezing, tremor etc.), support that the sample of LLD patients is not likely a sample of subclinical PD patients and support the ability of the voxel based analysis method described here to differentiate between motor disorders that have similar symptomatology.

The voxel based analysis method is able to differentiate between PD and related motor disorders. In our study, the voxel based analysis method was able to determine key differences between the subregions of the SNc that are impacted by late life depression (LLD) and regions that are known to be impacted in PD. While both LLD and PD show psychomotor slowing, the voxel based analysis method was able to determine that the subregions of the SNc impacted by LLD are different from those known to be impacted by PD pathology. The voxel based analysis method was able to determine that the patients with LLD did not represent PD. This provides strong evidence that the voxel based analysis method can differentiate between different movement disorders that have been shown to have overlapping symptoms. This can be applied to help guide the diagnosis of PD and rule out related disorders with similar presentation

The voxel based analysis of baseline NM-MRI symptom associated voxels may predict future response to treatment and provide an important prognostic biomarker for PD

In a secondary analysis of a smaller sample of subjects who underwent L-DOPA treatment, a nonsignificant trend toward a positive association between baseline NM-MRI and changes in psychomotor speed after treatment were observed. Although our data did not reach significance it was severely underpowered and we expect this to reach significance in a larger study.

This analysis suggests that the individual baseline NM-MRI voxels that the voxel based analysis determines are associated with a symptom of a motor disorder, may be able to predict a future response to treatment for that symptom before treatment has been initiated. This would provide critical prognostic information that could predict the course of a disease and guide the selection of the appropriate treatment. Since psychomotor slowing is a hallmark symptom of Parkinson's disease and L-DOPA treatment is one of the most effective and widely used PD therapies, this finding directly supports application of the voxel based analysis method to prediction of response to treatment for the motor symptoms of Parkinson's disease, including psychomotor slowing.

To Monitor Response to Treatment

Furthermore, it was observed that 3-week L-DOPA treatment, one of the most widely used PD treatments, was associated with significant increases in NM-MRI signal. This is the first evidence to date that administration of L-DOPA therapy induces changes in the substantia nigra that can be measured via voxel based analysis of NM-MRI. This was supported by previous work showing that NM-MRI captures NM concentration in ex vivo tissue samples and that it correlates with increased dopamine transmission, consistent with the finding that enhancing dopamine synthesis results in increased NM accumulation.

This supports the ability of the voxel based analysis method to track changes in NM-MRI data over the course of therapy. This provides a non-invasive method to track response to therapy and determine if a patient has had an adequate or inadequate response to therapy. This information can be used to guide therapy and help determine whether to increase or decrease the dosage of therapy. In this case, in a patient with Parkinson's disease, Parkinson's-disease-voxels should respond to treatment with L-DOPA. For example, the administration of L-DOPA to a patient with established Parkinson's-disease-voxels should cause a change in the Parkinson's-disease-voxel relative to the
The patient's baseline Parkinson's-disease-voxel reading
The reading from an untreated patient with similar Parkinson's-disease-voxels
and relative to a standard control

The change in the Parkinson's-disease-voxels could indicate if a patient has been adequately treated. This method may be applied to therapeutics other than L-DOPA.

Using the voxel based analysis method to detect differences between patients that can be used to predict different responses to treatment unique to each patient

In an exploratory analysis, a significant increase in NM-MRI signal after L-DOPA treatment was observed, supporting the notion that the L-DOPA treatment is likely increasing available striatal dopamine, but that participants are responding differently to that increase. This is important because it shows that the voxel based analysis method can detect differences in response to treatment that are specific to each patient. It is unlikely that the observed changes are due to natural NM accumulation over time, because this age-related process occurs very slowly and should only be detectable over a substantially longer timescale than the 3-week period evaluated here. Furthermore, although the sample size is limited (N=6), the excellent reproducibility of NM-MRI suggests that any observed increase in NM-MRI signal is indeed due to an increase in NM concentration. This result provides further evidence supporting that NM-MRI measures dopamine function, including synthesis induced by L-DOPA. This result also suggests that NM-MRI may be surprisingly sensitive to changes in NM at shorter timescales than previously thought. This finding suggests that NM-MRI could be well suited for monitoring of dopaminergic treatment response in patients with PD or related disorders.

In conclusion, in patients with LLD, an association was found between NM-MRI signal in the SN-VTA and baseline gait speed, and a trend level effect with changes in gait speed or processing speed after 3 weeks of L-DOPA treatment.

### REFERENCES FOR EXAMPLE 4

Peterson D S, Mancini M, Fino P C, Horak F, Smulders K (2020) Speeding Up Gait in Parkinson's Disease. Journal of Parkinson's Disease 10: 245-253.
Friedhoff A J, Ballenger J, Bellack A S, Carpenter W T, Jr, Chui H C, Dobrof R, et al. Diagnosis and Treatment of Depression in Late Life. JAMA. 1992; 268(8):1018-24.
Rothschild A J. The diagnosis and treatment of late-life depression. The Journal of clinical psychiatry. 1996; 57:5-11.
Alexopoulos G S, Meyers B S, Young R C, Kakuma T, Feder M, Einhorn A, et al. Recovery in geriatric depression. Archives of General Psychiatry. 1996; 53(4):305-12.
Sneed J R, Rutherford B R, Rindskopf D, Lane D T, Sackeim H A, Roose S P. Design makes a difference: a meta-analysis of antidepressant response rates in placebo-controlled versus comparator trials in late-life depression. The American Journal of Geriatric Psychiatry. 2008; 16(1):65-73.
Sheline Y I, Barch D M, Garcia K, Gersing K, Pieper C, Welsh-Bohmer K, et al. Cognitive function in late life depression: relationships to depression severity, cerebrovascular risk factors and processing speed. Biological psychiatry. 2006; 60(1):58-65.
Brown P J, Roose S P, Zhang J, Wall M, Rutherford B R, Ayonayon H N, et al. Inflammation, depression, and slow gait: a high mortality phenotype in later life. Journals of Gerontology Series A: Biomedical Sciences and Medical Sciences. 2016; 71(2):221-7.
Rutherford B R, Taylor W D, Brown P J, Sneed J R, Roose S P. Biological aging and the future of geriatric psychiatry. Journals of Gerontology Series A: Biomedical Sciences and Medical Sciences. 2017; 72(3):343-52.
Pimontel M A, Culang-Reinlieb M E, Morimoto S S, Sneed J R. Executive dysfunction and treatment response in late-life depression. International journal of geriatric psychiatry. 2012; 27(9):893-9.
Kerse N, Flicker L, Pfaff J J, Draper B, Lautenschlager N T, Sim M, et al. Falls, depression and antidepressants in later life: a large primary care appraisal. PLoS One. 2008; 3 (6).
Wolinsky F D, Callahan C M, Fitzgerald J F, Johnson R J. Changes in functional status and the risks of subsequent nursing home placement and death. Journal of gerontology. 1993; 48 (3):S94-101.
Rutherford B R, Slifstein M, Chen C, Abi-Dargham A, Brown P J, Wall M W, et al. Effects of L-DOPA Monotherapy on Psychomotor Speed and [11C] Raclopride Binding in High-Risk Older Adults With Depression. Biological psychiatry. 2019; 86(3):221-9.
Rapp M A, Reischies F M. Attention and executive control predict Alzheimer disease in late life: results from the Berlin Aging Study (BASE). The American Journal of Geriatric Psychiatry. 2005; 13(2):134-41.
Verghese J, Holtzer R, Lipton R B, Wang C. Quantitative gait markers and incident fall risk in older adults. The Journals of Gerontology: Series A. 2009; 64(8):896-901.
Guralnik J M, Ferrucci L, Pieper C F, Leveille S G, Markides K S, Ostir G V, et al. Lower extremity function and subsequent disability: consistency across studies, predictive models, and value of gait speed alone compared with the short physical performance battery. The Journals of Gerontology Series A: Biological Sciences and Medical Sciences. 2000; 55 (4):M221-M31.
Bäckman L, Nyberg L, Lindenberger U, Li S C, Farde L. The correlative triad among aging, dopamine, and cognition: current status and future prospects. Neuroscience & Biobehavioral Reviews. 2006; 30(6):791-807.
Volkow N D, Gur R C, Wang G J, Fowler J S, Moberg P J, Ding Y S, et al. Association between decline in brain dopamine activity with age and cognitive and motor impairment in healthy individuals. Am J Psychiat. 1998; 155(3):344-9.
Kaasinen V, Vilkman H, Hietala J, Någren K, Helenius H, Olsson H, et al. Age-related dopamine D2/D3 receptor loss in extrastriatal regions of the human brain. Neurobiology of aging. 2000; 21(5):683-8.
Eckart C, Bunzeck N. Dopamine modulates processing speed in the human mesolimbic system. Neuroimage. 2013; 66:293-300.
Chain R, Studenski S, Perera S, Bohnen N. Striatal dopaminergic denervation and gait in healthy adults. Experimental brain research. 2008; 185(3):391-8.
Cassidy C M, Zucca F A, Girgis R R, Baker S C, Weinstein J J, Sharp M E, et al. Neuromelanin-sensitive MRI as a noninvasive proxy measure of dopamine function in the human brain. Proceedings of the National Academy of Sciences. 2019; 116(11):5108-17.
Chen X, Huddleston D E, Langley J, Ahn S, Barnum C J, Factor S A, et al. Simultaneous imaging of locus coeruleus and substantia nigra with a quantitative neuromelanin MRI approach. Magnetic resonance imaging. 2014; 32(10):1301-6.
Zucca F A, Basso E, Cupaioli F A, Ferrari E, Sulzer D, Casella L, et al. Neuromelanin of the human substantia nigra: an update. Neurotoxicity research. 2014; 25(1):13-23.
Zecca L, Shima T, Stroppolo A, Goj C, Battiston G, Gerbasi R, et al. Interaction of neuromelanin and iron in substantia nigra and other areas of human brain. Neuroscience. 1996; 73(2):407-15.
Zecca L, Bellei C, Costi P, Albertini A, Monzani E, Casella L, et al. New melanic pigments in the human brain that accumulate in aging and block environmental toxic metals. Proceedings of the National Academy of Sciences. 2008; 105(45):17567-72.
Sulzer D, Zecca L. Intraneuronal dopamine-quinone synthesis: a review. Neurotoxicity research. 1999; 1(3):181-95.
Wechsler D. The Wechsler Memory Scale, San Antonio, Ill., Psychological Corp. Harcourt; 1997.
Wengler K, He X, Abi-Dargham A, Horga G. Reproducibility assessment of neuromelanin-sensitive magnetic resonance imaging protocols for region-of-interest and voxelwise analyses. NeuroImage. 2020; 208:116457.
Slinker B K, Glantz S A. Multiple linear regression: accounting for multiple simultaneous determinants of a continuous dependent variable. Circulation. 2008; 117(13):1732-7.
Eklund A, Nichols T E, Knutsson H. Cluster failure: Why fMRI inferences for spatial extent have inflated false-positive rates. Proceedings of the national academy of sciences. 2016; 113(28):7900-5.
Hupfeld K E, Vaillancourt D E, Seidler R D. Genetic markers of dopaminergic transmission predict performance for older males but not females. Neurobiology of aging. 2018; 66:180.e11-.e21.
Rosano C, Metti A L, Rosso A L, Studenski S, Bohnen N I. Influence of Striatal Dopamine, Cerebral Small Vessel Disease, and Other Risk Factors on Age-Related Parkinsonian Motor Signs. The Journals of Gerontology: Series A. 2019; 75(4):696-701.
Rosso A L, Bohnen N I, Launer L J, Aizenstein H J, Yaffe K, Rosano C. Vascular and dopaminergic contributors to mild parkinsonian signs in older adults. Neurology. 2018; 90 (3):e223-e9.
Clark B C, Woods A J, Clark L A, Criss C R, Shadmehr R, Grooms D R. The Aging Brain & the Dorsal Basal Ganglia: Implications for Age-Related Limitations of Mobility. Advances in Geriatric Medicine and Research. 2019; 1 (2):e190008.
Salthouse T A. Aging and measures of processing speed. Biological psychology. 2000; 54 (1-3):35-54.
Nyberg L, Karalija N, Salami A, Andersson M, Wåhlin A, Kaboovand N, et al. Dopamine D2 receptor availability is linked to hippocampal-caudate functional connectivity and episodic memory. Proceedings of the National Academy of Sciences. 2016; 113(28):7918-23.
Vriend C, van Balkom T D, van Druningen C, Klein M, van der Werf Y D, Berendse H W, et al. Processing speed is related to striatal dopamine transporter availability in Parkinson's disease. NeuroImage: Clinical. 2020:102257.
Jaeger J. Digit symbol substitution test: the case for sensitivity over specificity in neuropsychological testing. Journal of clinical psychopharmacology. 2018; 38(5):513.
Seidler R D, Bernard J A, Burutolu T B, Fling B W, Gordon M T, Gwin J T, et al. Motor control and aging: links to age-related brain structural, functional, and biochemical effects. Neuroscience & Biobehavioral Reviews. 2010; 34(5):721-33.
Corti E J, Johnson A R, Riddle H, Gasson N, Kane R, Loftus A M. The relationship between executive function and fine motor control in young and older adults. Human movement science. 2017; 51:41-50.
LeMoult J, Gotlib I H. Depression: A cognitive perspective. Clinical Psychology Review. 2019; 69:51-66.
Li C T, Lin C P, Chou K H, Chen I Y, Hsieh J C, Wu C L, et al. Structural and cognitive deficits in remitting and non-remitting recurrent depression: a voxel-based morphometric study. Neuroimage. 2010; 50(1):347-56.
Yaroslavsky I, Allard E S, Sanchez-Lopez A. Can't look away: Attention control deficits predict rumination, depression symptoms and depressive affect in daily life. Journal of Affective Disorders. 2019; 245:1061-9.
Shura R D, Rowland J A, Martindale S L, Brearly T W, Delahanty M B, Miskey H M. Evaluating the motor slowing hypothesis of depression. Psychiatry research. 2017; 252:188-95.
Zecca L, Fariello R, Riederer P, Sulzer D, Gatti A, Tampellini D. The absolute concentration of nigral neuromelanin, assayed by anew sensitive method, increases throughout the life and is dramatically decreased in Parkinson's disease. FEBS letters. 2002; 510(3):216-20.
Tison F, Mons N, Geffard M, Henry P. The metabolism of exogenous L-dopa in the brain: an immunohistochemical study of its conversion to dopamine in non-catecholaminergic cells of the rat brain. Journal of neural transmission-Parkinson's disease and dementia section. 1991; 3(1):27-39.
Matsuura K, Maeda M, Tabei K i, Umino M, Kajikawa H, Satoh M, et al. A longitudinal study of neuromelanin-sensitive magnetic resonance imaging in Parkinson's disease. Neuroscience letters. 2016; 633:112-7.
Sulzer D, Cassidy C, Horga G, Kang U J, Fahn S, Casella L, et al. Neuromelanin detection by magnetic resonance imaging (MRI) and its promise as a biomarker for Parkinson's disease. NPJ Parkinson's disease. 2018; 4(1):11.
Damier P, Hirsch E, Agid Y, Graybiel A. The substantia nigra of the human brain: II. Patterns of loss of dopamine-containing neurons in Parkinson's disease. Brain. 1999; 122(8):1437-48.
Fearnley J M, Lees A J. Ageing and Parkinson's disease: substantia nigra regional selectivity. Brain. 1991; 114(5):2283-301.
Ofori E, Pasternak O, Planetta P J, Burciu R, Snyder A, Febo M, et al. Increased free water in the substantia nigra of Parkinson's disease: a single-site and multi-site study. Neurobiology of aging. 2015; 36(2):1097-104.
Planetta P J, Ofori E, Pasternak O, Burciu R G, Shukla P, DeSimone J C, et al. Free-water imaging in Parkinson's disease and atypical parkinsonism. Brain. 2016; 139(2):495-508.
Xiang Y, Gong T, Wu J, Li J, Chen Y, Wang Y, et al. Subtypes evaluation of motor dysfunction in Parkinson's disease using neuromelanin-sensitive magnetic resonance imaging. Neuroscience letters. 2017; 638:145-50.
Sulzer D, Bogulavsky J, Larsen K E, Behr G, Karatekin E, Kleinman M H, et al. Neuromelanin biosynthesis is driven by excess cytosolic catecholamines not accumulated by synaptic vesicles. Proceedings of the National Academy of Sciences. 2000; 97(22):11869-74.
Cebrián C, Zucca F A, Mauri P, Steinbeck J A, Studer L, Scherzer C R, et al. MHC-I expression renders catecholaminergic neurons susceptible to T-cell-mediated degeneration. Nature communications. 2014; 5:3633.
Zucca F A, Segura-Aguilar J, Ferrari E, Muñoz P, Paris I, Sulzer D, et al. Interactions of iron, dopamine and neuromelanin pathways in brain aging and Parkinson's disease. Progress in neurobiology. 2017; 155:96-119.
Zecca L, Casella L, Albertini A, Bellei C, Zucca F A, Engelen M, et al. Neuromelanin can protect against iron-mediated oxidative damage in system modeling iron overload of brain aging and Parkinson's disease. Journal of neurochemistry. 2008; 106(4):1866-75.
Zecca L, Wilms H, Geick S, Claasen J H, Brandenburg L O, Holzknecht C, et al. Human neuromelanin induces neuroinflammation and neurodegeneration in the rat substantia nigra: implications for Parkinson's disease. Acta neuropathologica. 2008; 116(1):47-55.
Rutherford B R, Wall M M, Brown P J, Choo T-H, Wager T D, Peterson B S, et al. Patient expectancy as a mediator of placebo effects in antidepressant clinical trials. Am J Psychiat. 2017; 174(2):135-42.

Computer Based Analysis

Exemplary procedures in accordance with the disclosure described herein can be performed by a cloud-based processing arrangement and/or a computing arrangement (e.g., computer hardware arrangement). Such processing/computing arrangement can be, for example entirely or a part of, or include, but not limited to, a computer/processor that can include, for example one or more microprocessors, and use instructions stored on a computer-accessible medium (e.g., RAM, ROM, hard drive, or other storage device).

For example a computer-accessible medium (e.g., as described herein above, a storage device such as an encrypted cloud file, hard disk, floppy disk, memory stick, CD-ROM, RAM, ROM, etc., or a collection thereof) can be provided (e.g., in communication with the processing arrangement). The computer-accessible medium can contain executable instructions thereon. In addition or alternatively, a storage arrangement can be provided separately from the computer-accessible medium, which can provide the instructions to the processing arrangement so as to configure the processing arrangement to execute certain exemplary procedures, processes, and methods, as described herein above, for example.

Further, the exemplary processing arrangement can be provided with or include an input/output ports, which can include, for example a wired network, a wireless network, the internet, an intranet, a data collection probe, a sensor, etc. The exemplary processing arrangement can be in communication with an exemplary display arrangement, which, according to certain exemplary embodiments of the present disclosure, can be a touch-screen configured for inputting information to the processing arrangement in addition to outputting information from the processing arrangement, for example. Further, the exemplary display arrangement and/or a storage arrangement can be used to display and/or store data in a user-accessible format and/or user-readable format.

### EQUIVALENTS

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

1. An in vivo method of determining the progression of Parkinson's disease over time in a subject, said method comprising:
(i) obtaining a first Neuromelanin-Magnetic Resonance Imaging (NM-MRI) scan at a first time point;
(ii) after step (i), obtaining a second NM-MRI scan at a second time point;
(iii) comparing the first neuromelanin magnetic resonance image to said second neuromelanin magnetic resonance image thereby determining whether a change in the level, signal and/or concentration of neuromelanin occurred between said first time point and said second time point.

2. The method according to claim 1, wherein if the change in the level, signal and/or concentration of neuromelanin at the second time point is more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 20%, or more than about 25% less than the level, signal and/or concentration of neuromelanin at the first time point, Parkinson's disease is progressing.

3. An in vivo method of diagnosing Parkinson's disease, said method comprising:
(i) obtaining a first neuromelanin magnetic resonance image at a first time point;
(ii) after step (i), obtaining a second neuromelanin magnetic resonance image at a second time point;
(iii) comparing the first neuromelanin magnetic resonance image to said second neuromelanin magnetic resonance image thereby determining whether a change in the level, signal and/or concentration of neuromelanin occurred between said first time point and said second time point.

4. The method according to any preceding claim, wherein if the change in the level, signal and/or concentration of neuromelanin at the second time point is more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 20%, or more than about 25% less than the level, signal and/or concentration of neuromelanin at the first time point, a diagnosis of Parkinson's disease is provided.

5. A method of diagnosing a patient with Parkinson's disease, said method comprising:
(i) measuring a level of neuromelanin
(ii) comparing the level of neuromelanin to a standard control,
(iii) optionally providing a diagnosis of Parkinson's disease if the measured level of neuromelanin is lower relative to the standard control.

6. The method according to any preceding claim, further comprising determining a first signal intensity from said first neuromelanin magnetic resonance image and determining a second signal intensity from said second neuromelanin magnetic resonance image, wherein said comparing the first magnetic resonance image to said second magnetic resonance image comprises comparing the first signal intensity to the second signal intensity.

7. The method of any of the preceding claims, wherein a standard control is a level of neuromelanin present at approximately the same levels in a population of subjects, or said standard control is approximately the average level of neuromelanin present in a population of subjects.

8. The method according to any preceding claim, wherein a neuromelanin gradient phantom is used to measure the level, signal and/or concentration of neuromelanin.

9. The method according to any preceding claim, wherein a neuromelanin phantom concentration gradient is scanned about once per patient, about once an hour, about once a day, about once a week, or about once a month.

10. The method of any of the preceding claims, wherein a neuromelanin phantom gradient is scanned daily.

11. The method according to any preceding claim, wherein a neuromelanin phantom gradient is scanned with each patient.

12. The method according to claims 5-11, wherein if the change in the level, signal and/or concentration of neuromelanin at the second time point is more than about 5% less or more than about 10% less than the level, signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Parkinson's disease is provided.

13. The method according to any preceding claim, wherein if the change in the level, signal and/or concentration of neuromelanin at the second time point is more than about 35% less, more than about 40% less, more than about 45% less, or more than about 50% less signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Parkinson's disease is provided.

14. The method according to any preceding claim, wherein the second time point is about 3 months, about 6 months, about 9 months, about 12 months, about 2 years, about 3 years, about 4 years, about 5 years, about 6 years, about 7 years, about 8 years, about 9 years, about 10 years, about 15 years, about 20 years, about 25 years, or about 30 years after the first time point.

15. A method of assessing the neuromelanin concentration in a region of interest of the brain of a subject comprising:
performing a Neuromelanin-Magnetic Resonance Imaging (NM-MRI) scan on the subject;
acquiring a neuromelanin dataset from the NM-MRI scan;
optionally encrypting the neuromelanin dataset;
uploading the neuromelanin dataset to a remote server;
optionally decrypting the dataset;
performing an analysis of the neuromelanin dataset, wherein the analysis comprises one or more of:
(i) comparing the neuromelanin dataset with one or more previously acquired neuromelanin datasets from the said subject;
(ii) comparing the neuromelanin dataset with a control dataset;
(iii) comparing the neuromelanin dataset with one or more previously acquired neuromelanin datasets from different subjects;
generating a report comprising the neuromelanin analysis;
optionally encrypting the report;
uploading the report to remote server; and
optionally decrypting the report.

16. A method of determining if a subject has or is at risk of developing Parkinson's disease, the method comprising analyzing one or more Neuromelanin-Magnetic Resonance Imaging (NM-MRI) scans of the subject's brain region of interest, wherein the analyzing comprises:
receiving imaging information of the brain region of interest; and
determining a NM concentration in the brain region of interest using voxelwise analysis based on the imaging information;
wherein the determining if a subject has or is at risk of developing Parkinson's disease comprises:
(1) if the one or more NM-MRI scans has a decreased NM signal compared to a one or more control scans without Parkinson's disease then the subject has or is at risk of developing Parkinson's disease; or
(2) if the one or more NM-MRI scans has a NM signal comparable to the signal of a one or more control scans without Parkinson's disease then the subject does not have or is not at risk of developing Parkinson's disease.

17. A method of treating a subject with Parkinson's disease, the method comprising analyzing Neuromelanin-Magnetic Resonance Imaging (NM-MRI) scans of the subject's brain region of interest, wherein the analyzing comprises:
(i) receiving imaging information of the brain region of interest at a first time point;
(ii) receiving imaging information of the brain region of interest at a second time point;
(iii) determining a NM concentration at the first and second time points in the brain region of interest using voxelwise analysis based on the imaging information; and
(iv) comparing the NM concentration at the first time point to the second time point, wherein the treatment method further comprises:
(1) if the NM-MRI scan at the second time point has a decreased NM signal compared to the NM signal at the first time point, the method comprises administering one or more of levodopa and carbidopa; or
(2) if the NM-MRI scan at the second time point has an increased NM signal compared to the NM signal at the first time point, the method comprises:
(a) withholding administering one or more of levodopa and carbidopa; and
(b) repeating steps (i) through (iv).

18. The method according to any preceding claim, wherein the MRI scan is neuromelanin sensitive.

19. A method of providing a treatment regimen to a patient comprising performing the NM-MRI scan, acquiring NM signal from the NM-MRI scan in a region of interest, comparing the NM signal from the NM-MRI scan in a region of interest data to age matched database numbers, if the NM signal is less than a pre-determined value, administering a corresponding treatment regimen.

21. The method according to any preceding claim, wherein the patient displays symptoms of Alzheimer's disease.

22. The method according to any preceding claim, wherein the NM-MRI scan distinguishes between Alzheimer's disease and Parkinson's disease.

23. The method according to any preceding claim, wherein the subject or patient exhibits one or more symptom of Parkinson's disease.

24. The method according to any preceding claim, wherein a patient is diagnosed with Parkinson's disease without displaying symptoms.

25. The method according to any preceding claim, wherein the NM-MRI distinguishes between Alzheimer's disease and Parkinson's disease.

25. The method according to any preceding claim, further comprising diagnosing the patient as having Parkinson's disease or as not having Parkinson's disease; and indicating the diagnosis to a user via a user interface.

26. The method according to any preceding claim, wherein the analysis is a voxelwise analysis.

27. The method according to any preceding claim, wherein the voxelwise analysis comprises determining at least one topographical pattern within the brain region of interest.

28. The method according to any preceding claim, wherein the method further comprises a calculation using a value that represents a volume of a neuromelanin voxel.

29. The method according to any preceding claim, wherein the voxelwise analysis region of interest is the substantia nigra.

30. The method according to any preceding claim, wherein the voxelwise analysis region of interest the ventral substantia nigra subregion.

31. A diagnostic system for providing diagnostic information for Parkinson's disease, the diagnostic system comprising:
an MRI system configured to generate and acquire a neuromelanin sensitive MRI scan along with a neuromelanin data series for a voxel located within a region of interest in a subject's brain;
a signal processor configured to process the series of neuromelanin data to produce a processed neuromelanin MRI spectrum; and
a diagnostic processor configured to process the processed neuromelanin MRI spectrum to:
extract a measurement from the region of interest corresponding with neuromelanin at a time point,
compare the measurement to one or more control measurements acquired prior to the time point;
provide a diagnosis of Parkinson's disease if the measurement is more than about 25% less than the control measurement.

32. A method for treating a patient with Parkinson's disease comprising:
a) administering to a patient an initial amount of L-dopa;
b) performing serial NM-MRI scans of the patient monitoring the neuromelanin concentration in a region of interest in the patient's brain and assessing treatment-related adverse events over an initial treatment period;
c) if, during the initial treatment period, the patient exhibits
i) decreased neuromelanin concentration in the region of interest in the patient's brain;
ii) no L-dopa associated adverse or side effects;
then increasing the dose of L-dopa in a subsequent treatment period;
wherein the L-dopa treatment results in an improvement in Parkinson's disease symptoms in the patient.

33. The method of claim 32, including the following step:
d) repeating steps a)-c) until the patient fails to exhibit one or more of i)-ii) in step c).

34. The method according to any preceding claim, wherein the method is used with a second imaging method, wherein the second imaging method is selected from the group consisting of positron emission tomography (PET), structural MRI, comprises functional MRI (fMRI), blood oxygen level dependent (BOLD) fMRI, iron sensitive MRI, quantitative susceptibility mapping (QSM), diffusion tensor imaging DTI, and single photon emission computed tomography (SPECT), DaTscan and DaTquant.

35. The method according to any preceding claim, wherein the second imaging method comprises Positron Emission Tomography (PET).

36. The method according to any preceding claim, wherein the second imaging method comprises structural MRI.

37. The method according to any preceding claim, wherein the second imaging method comprises functional MRI (fMRI).

38. The method according to any preceding claim, wherein the second imaging method comprises blood oxygen level dependent (BOLD) fMRI.

39. The method according to any preceding claim, wherein the voxelwise analysis comprises determining at least one topographical pattern within the brain region of interest, wherein the brain region of interest is one or more Parkinson's disease symptom-associated voxels.

40. The method according to any preceding claim, wherein the voxelwise analysis comprises determining at least one topographical pattern within the brain region of interest, wherein the brain region of interest is one or more patient specific Parkinson's disease symptom-associated voxels.

41. The method according to any preceding claim, wherein the brain region of interest is the substantia nigra or the locus coeruleus.

42. The method according to claims 1-41, wherein the brain region of interest is the ventral substantia nigra.

43. The method according to claims 1-41, wherein the brain region of interest is the lateral substantia nigra.

44. The method according to claims 1-41, wherein the brain region of interest is the ventrolateral substantia nigra.

45. The method according to claims 1-41, wherein the brain region of interest is the substantia nigra pars Compacta (SNpc).

46. The method according to claims 1-41, wherein the brain region of interest is the substantia nigra pars reticulata (SNpr).

47. The method according to claims 1-41, wherein the brain region of interest is the ventral tegmental area (VTA).

48. The method according to claims 1-41, wherein the brain region of interest is the locus coeruleus.
